SEQUENCE LISTING (124 sequences)

SEQ ID NO: 1 - DNA, 2201 bp, Homo sapiens
ggccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacctgccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatttgtcaagagtagttatataattgttgaacaggtgtttttccacaagtgccgcaaattgtacctttttttttttttcaaaatagaaaagttattagtggtttatcagcaaaaaagtccaattttaatttagtaaatgttattttatactgtacaataaaaacattgcctttgaatgttaattttttggtacaaaaataaatttatatgaaaaaaaaaaaaaaaaaaaaaaaaaaaaaaaa

SEQ ID NO: 2 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 3 - DNA, 2238 bp, Mus musculus
ccccacgtcccggggagccactgccaggacctttgtgaaccggtcggggcgggggccgtggcggagtctgctcggcggtgggtggcccgagaagggagagaacgatcgcggagcagggcgcccgaactccgggcgccgcgccatgcgccgggccagccgagactacggcaagtacctgcgcagctcggaagagatgggcagcggccccggcgtcccacacgaaggtccgctgcaccccgcgccttctgcaccggctccggcgccgccacccgccgcctcccgctccatgttcctggccctcctggggctgggactgggccaggtggtctgcagcatcgctctgttcctgtactttcgagcgcagatggatcctaacagaatatcagaagacagcactcactgcttttatagaatcctgagactccatgaaaacgcaggtttgcaggactcgactctggagagtgaagacacactacctgactcctgcaggaggatgaaacaagcctttcagggggccgtgcagaaggaactgcaacacattgtggggccacagcgcttctcaggagctccagctatgatggaaggctcatggttggatgtggcccagcgaggcaagcctgaggcccagccatttgcacacctcaccatcaatgctgccagcatcccatcgggttcccataaagtcactctgtcctcttggtaccacgatcgaggctgggccaagatctctaacatgacgttaagcaacggaaaactaagggttaaccaagatggcttctattacctgtacgccaacatttgctttcggcatcatgaaacatcgggaagcgtacctacagactatcttcagctgatggtgtatgtcgttaaaaccagcatcaaaatcccaagttctcataacctgatgaaaggagggagcacgaaaaactggtcgggcaattctgaattccacttttattccataaatgttgggggatttttcaagctccgagctggtgaagaaattagcattcaggtgtccaacccttccctgctggatccggatcaagatgcgacgtactttggggctttcaaagttcaggacatagactgagactcatttcgtggaacattagcatggatgtcctagatgtttggaaacttcttaaaaaatggatgatgtctatacatgtgtaagactactaagagacatggcccacggtgtatgaaactcacagccctctctcttgagccctgtacaggttgtgtatatgtaaagtccataggtgatgttagattcatggtgattacacaacggttttacaattttgtaatgatttcctagaattgaaccagattgggagaggtattccgatgcttatgaaaaacttacacgtgagctatggaagggggtcacagtctctggtctaacccctggacatgtgccactgagaaccttgaaattaagaggatgccatgtcattgcatagaaatgatagtgtgaagggttaagttcttttgaattgttacattgcgctgggacctgcaaataagttctttttttctaatgaggagaaaaatatatgtatttttatataatgtctaaagttatatttcaggtgtaatgttttctgtgcaaagttttgtaaattatatttgtgctatagtatttgattcaaaatatttaaaaatgtctcactgttgacatatttaatgttttaaatgtacagatgtatttaactggtgcactttgtaattcccctgaaggtactcgtagctaagggggcagaatactgtttctggtgaccacatgtagtttatttctttattctttttaacttaatagagtcttcagacttgtcaaaactatgcaagcaaaataaataaataaaaataaaatgaataccttgaataataagtaggatgttggtcaccaggtgcctttcaaatttagaagctaattgactttaggagctgacatagccaaaaaggaacataataggctactgaaatctgtcaggagtatttatgcaattattgaacaggtgtctttttttacaagagctacaaattgtaaattttggtttcttttttttcccatagaaaatgtactatagtttatcagccaaaaaacaatccactttttaatttagtgaaagttattttattatactgtacaataaaagcattgtctctgaatgttaattttttggtacaaaaaataaatttgtacgaaaaaaaaaaaaaaaaaaaaaaaaa

SEQ ID NO: 4 - protein, 316 aa, Mus musculus
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 5 - DNA, 1769 bp, Homo sapiens
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 6 - protein, 281 aa, Homo sapiens [sequence omitted]

SEQ ID NO: 7 - DNA, 1377 bp, Homo sapiens
agcatcctgagtaatgagtggcctgggccggagcaggcgaggtggccggagccgtgtggaccaggaggagcgctttccacagggcctgtggacgggggtggctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagaagaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagtggccttggcattgtgtgtgtgcctgcccaggaggggggcccaggtgcataaatgggggtcagggagggaaaggaggagggagagagatggagaggaggggagagagaaagagaggtggggagaggggagagagatatgaggagagagagacagaggaggcagagagggagagaaacagaggagacagagagggagagagagacagagggagagagagacagaggggaagagaggcagagagggaaagaggcagagaaggaaagagacaggcagagaaggagagaggcagagagggagagaggcagagagggagagaggcagagagacagagagggagagagggacagagagagatagagcaggaggtcggggcactctgagtcccagttcccagtgcagctgtaggtcgtcatcacctaaccacacgtgcaataaagtcctcgtgcctgctgctcacagcccccgagagcccctcctcctggagaataaaacctttggcagctgcccttcctcaaaaaaaaaaaaaaaaaaaa

SEQ ID NO: 8 - protein, 250 aa, Homo sapiens [sequence omitted]

SEQ ID NO: 9 - DNA, 934 bp, Homo sapiens
agctcagcctcagtccccgcagcttgtgcggcggcgtcggcaccatgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatagcagggagccggcaggaggtggcccctgccctccctctggacccccagccaggggcttggaaatcaaattcagctcttcactccagcatgcacatgccctctttctgggaccaggctaactctgcagaagcacagacactacagaccacagcattcagcccccatggagtttggtgtgcttgcctttggcttcagacctcaccatctttgacagcccttgaaggtggtagcccagctcctgttcctgtgccttcaaaaggctggggcactatgagtaaaagaccgcttttaaaatggggaaggcaccattaagccaaaatgaatctgaaaaaagac

SEQ ID NO: 10 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 11 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 12 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 13 - DNA, 1979 bp, Homo sapiens
cccttgctgccgcctctcgcctcacgcccccagagaagagtttctccaccaggcagcaggtgaaggtttttttccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagggctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaacctctaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatacttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctgggacagccccctgttcctgagatggaaagcacaaatttaatacaccaccacaatggaaaacaagttcaaagacttttacttacagatcctggacagaaagggcataatgagtctgaagggcagtcctccttctctaggttacatgaggcaggaataagaagtcagacagagacagcaagacagttaacaatgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttctggtatgagaaattaaacttgtatacaaaatggttgccaaggcaacataaaattataagaattc

SEQ ID NO: 14 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 15 - DNA, 1834 bp, Homo sapiens
actttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaa

SEQ ID NO: 16 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 17 - DNA, 1909 bp, Homo sapiens
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

SEQ ID NO: 18 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 19 - DNA, 1000 bp, Homo sapiens
gaggttgaaggacccaggcgtgtcagccctgctccagagaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggctgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggac

SEQ ID NO: 20 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 21 - DNA, 1305 bp, Homo sapiens
cacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggccagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccacagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtca

SEQ ID NO: 22 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 23 - DNA, 1325 bp, Homo sapiens
gaggtttattgggcctcggtcctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggacccacactccctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagagggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctga

SEQ ID NO: 24 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 25 - DNA, 1485 bp, Mus musculus
catgccgagtgctttgtgtgtgttacctgctctaagaagctggctgggcagcgtttcaccgctgtggaggaccagtattactgcgtggattgctacaagaactttgtggccaagaagtgtgctggatgcaagaaccccatcactgggtttggtaaaggctccagtgtggtggcctatgaaggacaatcctggcacgactactgcttccactgcaaaaaatgctccgtgaatctggccaacaagcgctttgtatttcataatgagcaggtgtattgccctgactgtgccaaaaagctgtaacttgacggctgccctgtccttcctagataatggcaccaaattctcctgaggctaggggggaaggagtgtcagagtgtcactagctcgaccctggggacaagggggactaatagtaccctagcttgatttcttcctattctcaagttcctttttatttctcccttgcgtaacccgctcttcccttctgtgcctttgcctgtattcccaccctccctgctacctcttggccacctcacttctgagaccacagctgttggcagggtccctagctcatgccagcctcatctccaggccacatggggggctcagtcagagagccagccctttcggttgctctttggttgagttggggggcagttctgggggctgtgacttgtgctgtcgcactactgatccaacagacagagctgcaaagcctaaggcgggaggtgagccggctgcagcggagtggagggccttcccagaagcagggagagcgcccatggcagagcctctgggagcagagtcctgatgtcctggaagcctggaaggatggggcgaaatctcggagaaggagagcagtactcacccagaagcacaagaagaagcactcagtcctgcatcttgttccagttaacattacctccaaggactctgacgtgacagaggtgatgtggcaaccagtacttaggcgtgggagaggcctggaggcccagggagacattgtacgagtctgggacactggaatttatctgctctatagtcaggtcctgtttcatgatgtgactttcacaatgggtcaggtggtatctcgggaaggacaagggagaagagaaactctattccgatgtatcagaagtatgccttctgatcctgaccgtgcctacaatagctgctacagtgcaggtgtctttcatttacatcaaggggatattatcactgtcaaaattccacgggcaaacgcaaaacttagcctttctccgcatggaacattcctggggtttgtgaaactatgattgttataaagggggtggggatttcccattccaaaaactggctagacaaaggacaaggaacggtcaagaacagctctccatggctttgccttgactgttgttcctccctttgcctttcccgctcccactatctgggctttgactccatggatattaaaaaagtagaatattttggtttatctcccaaaaa

SEQ ID NO: 26 - protein, 240 aa, Mus musculus
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKDSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 27 - DNA, 1120 bp, Homo sapiens
ccgcaaggaaaacccagactctggcgacagcagagacgaggatgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctgacctgggcccaccaaggtggacgctgggccccgccaggctggagcccggagggtctgctgggcgagcagggcaggtgcaggccgcctgccccgccacgctcctgggccaactctgcaccgttctaggtgccgatggctgcctccggctctctgcttacgtatgccatgcatacctcctgccccgcgggaccacaataaaaaccttggcagacgggagtctccgaccggcaaaaaaaaaaaaaaaaa

SEQ ID NO: 28 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 29 - DNA, 748 bp, Homo sapiens
catgacattgcatccttcacccatcacttgtgaatttttgttttccacagctctcatttctccaaaaatgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctagagacttgatttgatctcctcattcccttcagcacatgtagaggtgccagtgggtggattggagggagaagatattcaatttctagagtttgtctgtctacaaaaatcaacacaaacagaactcctctgcacgtgaattttcatctat

SEQ ID NO: 30 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 31 - DNA, 5296 bp, Homo sapiens
attccctcggcgggccgagcctcccctctctcccgcccctcctcctccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtgggtgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagggagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacaaacagaattttccatttattaagcagtacaaatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtgctcatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctagagggcgagggatgatgagggaagcaataggtagctggggagccctattgctgctaagtcattggcaaagtgacaaagcaatttactgatgagagaatgtggaaatagatgtgcagtttggaattatgttggtgtgaatttgccagaggaccaatgcttgcatggagaatgggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcttttgctgaacaggaaagagctaggaaccctggaggtaaacaaagactttgatccatgtatgagtgtatgtgtttatgtaacttcctgtggatgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatggtttattctttgcaaataaaacgctttccccgtctgttcttgaaaaaaaaaaaaaaaaaa

SEQ ID NO: 32 - protein, 163 aa, Homo sapiens
GSHMGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 33 - DNA, 5290 bp, Homo sapiens
attccctcggcgggccgagcctcccctctctcccgcccctcctcctccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtgggtgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagggagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacaaacagaattttccatttattaagcagtacaaatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtgctcatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctagagggcgagggatgatgagggaagcaataggtagctggggagccctattgctgctaagtcattggcaaagtgacaaagcaatttactgatgagagaatgtggaaatagatgtgcagtttggaattatgttggtgtgaatttgccagaggaccaatgcttgcatggagaatgggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcttttgctgaacaggaaagagctaggaaccctggaggtaaacaaagactttgatccatgtatgagtgtatgtgtttatgtaacttcctgtggatgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatggtttattctttgcaaataaaacgctttccccgtctgttcttgaaaaaaaaaaaaaaaaaa

SEQ ID NO: 34 - protein, 389 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVYYINFTDFASYEVVVDEKPFLQCTRSIETGKTNYNTCYTAGVCLLKARQKIAVKMVHADISINMSKHTTFFGAIRLGEAPAS

SEQ ID NO: 35 - protein, 316 aa, Artificial Sequence
  murine RANKL variant D1-1 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWADISNMTLSNGKLRVNQDGFYYLYANISFRHHETSGSVPTDYLQLMVYVVKTSIKIPSSHNLMKGGSTKNWSGNSEFHYYSINVGGFFKLRAGEEISIQVSNPSLLDPDQDATYFGAFKVQDID

SEQ ID NO: 36 - protein, 316 aa, Artificial Sequence
  murine RANKL variant D1-1 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGYYYLYANICFRHHETSGSVPTDYLQLMVYVVKTSIKIPSSHNLMDGGSTKNWSGNSEFHFYSINVGGYFKLRAGEEISIQVSNPSLLDPDQDATYFGAFKVQDID

SEQ ID NO: 37 - protein, 316 aa, Artificial Sequence
  murine RANKL variant D2-0 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKLRVNQDGFYYLYANICFRHHETSGKVPTDYLQLMVYVVKTSIKIPSSHNLMKGGSTKNWSGNSEFHYYSINVGGFFKLRAGEEISIQVSNPSLLDPDQDATYFGAFKVQDID

SEQ ID NO: 38 - protein, 316 aa, Artificial Sequence
  murine RANKL variant D2-2 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKLRVNQDGYYYLYANIAFRHHETSGKVPTDYLQLMVYVVKTSIKIPSSHNLMDGGSTKNWSGNSEFHFYSINVGGYFKLRAGEEISIQVSNPSLLDPDQDATYFGAFKVQDID

SEQ ID NO: 39 - protein, 317 aa, Artificial Sequence
  human RANKL variant D1-1 monomer A
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWARISNMTFSNGKLIVNQDGFYYLYANISFRHHETSGDLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 40 - protein, 317 aa, Artificial Sequence
  human RANKL variant D1-1  monomer B
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGYYYLYANICFRHHETSGDLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 41 - protein, 317 aa, Artificial Sequence
  human RANKL variant D2-0 monomer A
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLVINATDIPSGSHKVSLSSWYHDRGWAKISNMTFSNGKLIVNQDGFYYLYANICFRHHETSGKLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 42 - protein, 317 aa, Artificial Sequence
  human RANKL variant D2-0 monomer B
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLVINATDIPSGSHKVSLSSWYHDRGWAKISNMTFSNGKLIVNQDGYYYLYANIAFRHHETSGKLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 43 - protein, 281 aa, Artificial Sequence
  human trail variant D1 monomer A
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSDLSNLHLRNGELVIHEKGFYYIYSQTSFRFQEEIKENTKNDKQMVQYIYKYTSYPDPILLMKSARNSCWSKDAEYGYYSIYQGGIFELKENDRIFVSVTNEHLIDMDHEASFFGAFLVG

SEQ ID NO: 44 - protein, 281 aa, Artificial Sequence
  human trail variant D1 monomer B
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGYYYIYSQTYFRFQEEIKENTKNDKQMVQYIYKYTSYPDPILLMDSARNSCWSKDAEYGLYSIYQGGYFELKENDRIFVSVTNEHLIDMDHEASFFGAFLVG

SEQ ID NO: 45 - protein, 281 aa, Artificial Sequence
  human trail variant D2 monomer A
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHIVGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGYYSIYQGGIFELKENDRIFVSVTNEHLIDMDHEASFFGAFLVG

SEQ ID NO: 46 - protein, 281 aa, Artificial Sequence
  (human trail variant D2 monomer B
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGVRGRSNTLSSPNSKNEKALGRKINSWESSRSGHSFLSNLHLRNGELVIHEKGYYYIYSQTAFRFQEEIKENTKNDKQMVQYIYKYTSYPDPILLMDSARNSCWSKDAEYGLYSIYQGGYFELKENDRIFVSVTNEHLIDMDHEASFFGAFLVG

SEQ ID NO: 47 - protein, 285 aa, Artificial Sequence
  human BAFF variant D1 monomer A
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSYKRGSALEEKENKILVKETGYFFIYGQVSYTDKTYAMGHLIQRKKVHVFGDELSLVTLFRCIQNMPETLPYNSCYSAGIAKLEEGDELQLAIPRENAQISLDGDVTFFGALKLL

SEQ ID NO: 48 - protein, 285 aa, Artificial Sequence
  human BAFF variant D1 monomer B
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFDCIQNMPETLPNNSCYSAGYAKLEEGDELQLAIPRENAQISLDGDVTFFGALKLL

SEQ ID NO: 49 - protein, 285 aa, Artificial Sequence
  human BAFF variant D2 monomer A
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLVADSETPTIQKGSYTFVPWLLSFKRGSALEEKENKILVKETGYFFIYGQVLYTDKTYAMGHLIQRKKVHVFGDELSLVTLFRCIQNMPETLPYNSCYSAGIAKLEEGDELQLAIPRENAQISLDGDVTFFGALKLL

SEQ ID NO: 50 - protein, 285 aa, Artificial Sequence
  (human BAFF variant D2 monomer B
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLDADSETPTIQKGSYTFVPWLLSFKRGSALEEKENKILVKETGYFFIYGQVAYTDKTYAMGHLIQRKKVHVFGDELSLVTLFDCIQNMPETLPNNSCYSAGYAKLEEGDELQLAIPRENAQISLDGDVTFFGALKLL

SEQ ID NO: 51 - protein, 240 aa, Artificial Sequence
  Murine APRIL variant D1 monomer A
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKDSDVTEVMWQPVDRRGRGLEAQGDIVRVWDTGIYLLYSQVSFHDVTFTMGQVVSREGQGRRETLFRCIRSMPSDPDRAYNSCYSAGVFHLHQGDIITVKIPRANAKLSLSPHGTFLGFVKL

SEQ ID NO: 52 - protein, 240 aa, Artificial Sequence
  Murine APRIL variant D1 monomer B
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKDSDVTEVMWQPVLRRGRGLEAQGDIVRVWDTGYYLLYSQVLFHDVTFTMGQVVSREGQGRRETLFDCIRSMPSDPDRAYNSCYSAGYFHLHQGDIITVKIPRANAKLSLSPHGTFLGFVKL

SEQ ID NO: 53 - protein, 240 aa, Artificial Sequence; identical to SEQ ID NO: 26
  Murine APRIL variant D2 monomer A

SEQ ID NO: 54 - protein, 240 aa, Artificial Sequence
  Murine APRIL variant D2 monomer B
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKDSDVTEVMWQPVLRRGRGLEAQGDIVRVWDTGYYLLYSQVAFHDVTFTMGQVVSREGQGRRETLFDCIRSMPSDPDRAYNSCYSAGYFHLHQGDIITVKIPRANAKLSLSPHGTFLGFVKL

SEQ ID NO: 55 - protein, 233 aa, Artificial Sequence
  TNFalpha variant D1 monomer A
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANASLANGVELRDNQLVVPSEGLYLIYSQVSFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 56 - protein, 233 aa, Artificial Sequence
  TNFalpha variant D1 monomer B
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGYYLIYSQVLFKGQGCPSTHVLLTHTIDRIAVSYQTKVNLLSAIKSPCQRETPEGAEAKPWYEPIYLGGYFQLEKGDRLSAEINRPDYLDFAESGQVYFGIIAL

SEQ ID NO: 57 - protein, 233 aa, Artificial Sequence; identical to SEQ ID NO: 12
  TNFalpha variant D2 monomer A

SEQ ID NO: 58 - protein, 233 aa, Artificial Sequence
  TNFalpha variant D2 monomer B
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGYYLIYSQVAFKGQGCPSTHVLLTHTIDRIAVSYQTKVNLLSAIKSPCQRETPEGAEAKPWYEPIYLGGYFQLEKGDRLSAEINRPDYLDFAESGQVYFGIIAL

SEQ ID NO: 59 - protein, 205 aa, Artificial Sequence
  TNFbeta variant D1 monomer A
MTPPERLFLPRVCGTTLHLLLLGLLLVLLPGAQGLPGVGLTPSAAQTARQHPKMHLAHSTLKPAAHLIGDPSKQNSLLWRANTDRADLQDGFSLSNNSLLVPTSGIYFVYSQVSFSGKAYSPKATSSPLYLAHEVQLFSSQYPFHVPLLSSQKMVYPGLQEPWYHSMYHGAAFQLTQGDQLSTHTDGIPHLVLSPSTVFFGAFAL

SEQ ID NO: 60 - protein, 205 aa, Artificial Sequence
  TNFbeta variant D1 monomer B
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGYYFVYSQVVFSGKAYSPKATSSPLYLAHEVQLFSSQYPFHVPLLDSQKMVYPGLQEPWLHSMYHGAYFQLTQGDQLSTHTDGIPHLVLSPSTVFFGAFAL

SEQ ID NO: 61 - protein, 205 aa, Artificial Sequence
  TNFbeta variant D2 monomer A
MTPPERLFLPRVCGTTLHLLLLGLLLVLLPGAQGLPGVGLTPSAAQTARQHPKMHLAHSTLKPAAHLVGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWYHSMYHGAAFQLTQGDQLSTHTDGIPHLVLSPSTVFFGAFAL

SEQ ID NO: 62 - protein, 205 aa, Artificial Sequence
  TNFbeta variant D2 monomer B
MTPPERLFLPRVCGTTLHLLLLGLLLVLLPGAQGLPGVGLTPSAAQTARQHPKMHLAHSTLKPAAHLVGDPSKQNSLLWRANTDRAFLQDGFSLSNNSLLVPTSGYYFVYSQVAFSGKAYSPKATSSPLYLAHEVQLFSSQYPFHVPLLDSQKMVYPGLQEPWLHSMYHGAYFQLTQGDQLSTHTDGIPHLVLSPSTVFFGAFAL

SEQ ID NO: 63 - protein, 261 aa, Artificial Sequence
  human CD40L variant D1 monomer A
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYDMSNNLVTLENGKQLTVKRQGLYYIYAQVDFCSNREASSQAPFIASLCLKSPGRFERILLRAANTHSSAKPCGYQSIHLGGVFELQPGASVFVNVTDPSQVSHGTGFTSFGLLKL

SEQ ID NO: 64 - protein, 261 aa, Artificial Sequence
  human CD40L variant D1 monomer B
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGYYYIYAQVTFCSNREASSQAPFIASLCLKSPGRFERILLDAANTHSSAKPCGQQSIHLGGYFELQPGASVFVNVTDPSQVSHGTGFTSFGLLKL

SEQ ID NO: 65 - protein, 261 aa, Artificial Sequence
  human CD40L variant D2 monomer A
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVVSEASSKTTSVLQWAEKGYYTMSNNLVTLENGKQLTVKRQGLYYIYAQVTFCSNREASSQAPFIASLCLKSPGRFERILLRAANTHSSAKPCGYQSIHLGGVFELQPGASVFVNVTDPSQVSHGTGFTSFGLLKL

SEQ ID NO: 66 - protein, 261 aa, Artificial Sequence
  human CD40L variant D2 monomer B
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVVSEASSKTTSVLQWAEKGYYTMSNNLVTLENGKQLTVKRQGYYYIYAQVAFCSNREASSQAPFIASLCLKSPGRFERILLDAANTHSSAKPCGQQSIHLGGYFELQPGASVFVNVTDPSQVSHGTGFTSFGLLKL

SEQ ID NO: 67 - protein, 250 aa, Artificial Sequence
  human APRIL variant D1 monomer A
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPADRRGRGLQAQGYGVRIQDAGVYLLYSQVSFQDVTFTMGQVVSREGQGRQETLFRCIRSMPSHPDRAYNSCYSAGVFHLHQGDILSVIIPRARAKLNLSPHGTFLGFVKL

SEQ ID NO: 68 - protein, 250 aa, Artificial Sequence
  human APRIL variant D1 monomer B
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGYYLLYSQVLFQDVTFTMGQVVSREGQGRQETLDRCIRSMPSHPDRAYNSCYSAGYFHLHQGDILSVIIPRARAKLNLSPHGTFLGFVKL

SEQ ID NO: 69 - protein, 250 aa, Artificial Sequence [sequence omitted]

SEQ ID NO: 70 - protein, 250 aa, Artificial Sequence
  human APRIL variant D2 monomer B
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGYYLLYSQVAFQDVTFTMGQVVSREGQGRQETLDRCIRSMPSHPDRAYNSCYSAGYFHLHQGDILSVIIPRARAKLNLSPHGTFLGFVKL

SEQ ID NO: 71 - protein, 183 aa, Artificial Sequence
  human OX40L variant D1 monomer A
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGDYLISLKGYFSQEVNISLHYQKDEEPLFSLKKVRSVNSLMVASLTYKDKVYLNVTTDNTSLDDFHVNGGELILIHQNPGEFCVL

SEQ ID NO: 72 - protein, 183 aa, Artificial Sequence
  human OX40L variant D1 monomer B
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMDASLTYKDKVYLNVTTDNTSLDDFHVNGGELILIHQNPGEFCVL

SEQ ID NO: 73 - protein, 183 aa, Artificial Sequence
  human OX40L variant D2 monomer A
MERVQPLEENVGNAARPRFERNKLLLVASVIQGLGLLLCFTYICLHFSALQVSHRYPRIQSIKVVFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 74 - protein, 183 aa, Artificial Sequence
  human OX40L variant D2 monomer B
MERVQPLEENVGNAARPRFERNKLLLVASVIQGLGLLLCFTYICLHFSALQVSHRYPRIQSIKVVFTEYKKEKGFILTSQKEDEIMKVQNNSVIINCDGFYLISLKGYFSQEVNISLHYQKDEEPLFALKKVRSVNSLMDASLTYKDKVYLNVTTDNTSLDDFHVNGGELILIHQNPGEFCVL

SEQ ID NO: 75 - protein, 177 aa, Artificial Sequence; identical to SEQ ID NO: 30
  human GITRL variant D1 monomer A

SEQ ID NO: 76 - protein, 177 aa, Artificial Sequence
  human GITRL variant D1 monomer B
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGDYLIYGQVAPNANYNDVAPFEVRLYKNKDMIQSLTNKSKIQNVGGTYELHVGDTIDLIFNSEHQVLKNNTYWGIILLANPQFIS

SEQ ID NO: 77 - protein, 177 aa, Artificial Sequence
  human GITRL variant D2 monomer A
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVYPNANYNDVAPFEVRLYKNKDMIQTLTNKSKIQNVGGTYELHVGDTIDLIFNSEHQVLKNNTYWGIILLANPQFIS

SEQ ID NO: 78 - protein, 177 aa, Artificial Sequence
  human GITRL variant D2 monomer B
MCLSHLENMPLSHSRTQGAQRSSWKLWLFCSIVMLLFLCSFSWLIFIFLQLETAKEPCMAKFVPLPSKWQMASSEPPCVNKVSDWKLEILQNGLYLIYGQVYPNANYNDVAPFEVRLYKNKDMIQALTNKSKIQNVGGDYELHVGDTIDLIFNSEHQVLKNNTYWGIILLANPQFIS

SEQ ID NO: 79 - protein, 316 aa, Artificial Sequence
  murine RANKL variant D1-2 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWADISNMTLSNGKRRVNQDGFYYLYANISFRHHETSGSVPTDYLQLMVYVVKTSIKIPSSHNLMKGGSTKNWSGNSEYHFYSINVGGFFKLRAGEEISIQVSNPSLLDPDQDATYFGAFKVQDID

SEQ ID NO: 80 - protein, 316 aa, Artificial Sequence
  murine RANKL variant D1-2 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKRRVNQDGYYYLYANICFRHHETSGSVPTDYLQLMVYVVKTSIKIPSSHNLMDGGSTKNWSGNSEFHFYSINVGGYFKLRAGEEISIQVSNPSLLDPDQDATYFGAFKVQDID

SEQ ID NO: 81 - protein, 316 aa, Artificial Sequence
  murine RANKL variant D1-3 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWADISNMTLSNGKLRVNQDGFYYLYANISFRHHETSGSVPTDYLQLMVYVVKTSEKIPSSHNLMKGGSTKNWSGNSEFHYYSINVGGFFKLRAGEEISIQVSNPSLLDPDQDATYFGAFKVQDID

SEQ ID NO: 82 - protein, 316 aa, Artificial Sequence
  murine RANKL variant D1-3 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGYYYLYANICFRHHETSGSVPTDYLQLMVYVVKTSEKIPSSHNLMDGGSTKNWSGNSEFHFYSINVGGYFKLRAGEEISIQVSNPSLLDPDQDATYFGAFKVQDID

SEQ ID NO: 83 - protein, 316 aa, Artificial Sequence
  murine RANKL variant D2-1 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKLRVNQDGFYYLYANISFRHHETSGKVPTDYLQLMVYVVKTSIKIPSSHNLMKGGSTKNWSGNSEFHYYSINVGGFFKLRAGEEISIQVSNPSLLDPDQDATYFGAFKVQDID

SEQ ID NO: 84 - protein, 158 aa, Artificial Sequence
  murine RANKL variant D2-1 monomer B
MEAQPFAHLVINAASIPSGSHKVTLSSWYHDRGWAKISNMTLSNGKLRVNQDGYYYLYANICFRHHETSGKVPTDYLQLMVYVVKTSIKIPSSHNLMDGGSTKNWSGNSEFHFYSINVGGYFKLRAGEEISIQVSNPSLLDPDQDATYFGAFKVQDID

SEQ ID NO: 85 - protein, 316 aa, Artificial Sequence
  murine RANKL variant D2-2 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKRRVNQDGFYYLYANISFRHHETSGKVPTDYLQLMVYVVKTSIKIPSSHNLMKGGSTKNWSGNSEFHYYSINVGGFFKLRAGEEISIQVSNPSLLDPDQDATYFGAFKVQDID

SEQ ID NO: 86 - protein, 316 aa, Artificial Sequence
  murine RANKL variant D2-2 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKRRVNQDGYYYLYANICFRHHETSGSVPTKYLQLMVYVVKTSIKIPSSHNLMDGGSTKNWSGNSEFHFYSINVGGYFKLRAGEEISIQVSNPSLLDPDQDATYFGAFKVQDID

SEQ ID NO: 87 - protein, 316 aa, Artificial Sequence
  murine RANKL variant D2-3 monomer A
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKLRVNQDGFYYLYANISFRHHETSGKVPTDYLQLMVYVVKTSEKIPSSHNLMKGGSTKNWSGNSEFHYYSINVGGFFKLRAGEEISIQVSNPSLLDPDQDATYFGAFKVQDID

SEQ ID NO: 88 - protein, 316 aa, Artificial Sequence
  murine RANKL variant D2-3 monomer B
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLVINAASIPSGSHKVTLSSWYHDRGWAKISNMTLSNGKLRVNQDGYYYLYANICFRHHETSGSVPTKYLQLMVYVVKTSEKIPSSHNLMDGGSTKNWSGNSEFHFYSINVGGYFKLRAGEEISIQVSNPSLLDPDQDATYFGAFKVQDID

SEQ ID NO: 89 - protein, 171 aa, Artificial Sequence
  Human RANKL variant D1-1 monomer A His tag
MGSSHHHHHHSQDPEAQPFAHLTINATDIPSGSHKVSLSSWYHDRGWADISNMTFSNGKLIVNQDGFYYLYANISFRHHETSGDLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 90 - protein, 171 aa, Artificial Sequence
  Human RANKL variant D1-1 monomer B His tag
MGSSHHHHHHSQDPEAQPFAHLTINATDIPSGSHKVSLSSWYHDRGWAKISNMTFSNGKLIVNQDGYYYLYANICFRHHETSGDLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 91 - protein, 158 aa, Artificial Sequence
  Human RANKL variant D1-2 monomer A
MEAQPFAHLTINATDIPSGSHKVSLSSWYHDRGWADISNMTFSNGKLRVNQDGFYYLYANISFRHHETSGDLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 92 - protein, 171 aa, Artificial Sequence
  Human RANKL variant D1-2 monomer A His tag
MGSSHHHHHHSQDPEAQPFAHLTINATDIPSGSHKVSLSSWYHDRGWADISNMTFSNGKLRVNQDGFYYLYANISFRHHETSGDLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 93 - protein, 158 aa, Artificial Sequence
  Human RANKL variant D1-2 monomer B
MEAQPFAHLTINATDIPSGSHKVSLSSWYHDRGWAKISNMTFSNGKLRVNQDGYYYLYANICFRHHETSGDLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 94 - protein, 171 aa, Artificial Sequence
  Human RANKL variant D1-2 monomer B His tag
MGSSHHHHHHSQDPEAQPFAHLTINATDIPSGSHKVSLSSWYHDRGWAKISNMTFSNGKLRVNQDGYYYLYANICFRHHETSGDLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 95 - protein, 158 aa, Artificial Sequence
  Human RANKL variant D1-3 monomer A
MEAQPFAHLTINATDIPSGSHKVSLSSWYHDRGWADISNMTFSNGKLIVNQDGFYYLYANISFRHHETSGDLATEYLQLMVYVTKTSEKIPSSHTLMKGGSTKYWSGNSEFHYYSINVGGFFKLRSGEEISIEVSNPSLLDPDQDATYFGAFKVRDID

SEQ ID NO: 96 - protein, 171 aa, Artificial Sequence
  Human RANKL variant D1-3 monomer A His tag
MGSSHHHHHHSQDPEAQPFAHLTINATDIPSGSHKVSLSSWYHDRGWADISNMTFSNGKLIVNQDGFYYLYANISFRHHETSGDLATEYLQLMVYVTKTSEKIPSSHTLMKGGSTKYWSGNSEFHYYSINVGGFFKLRSGEEISIEVSNPSLLDPDQDATYFGAFKVRDID

SEQ ID NO: 97 - protein, 158 aa, Artificial Sequence
  Human RANKL variant D1-3 monomer B
MEAQPFAHLTINATDIPSGSHKVSLSSWYHDRGWAKISNMTFSNGKLIVNQDGYYYLYANICFRHHETSGDLATEYLQLMVYVTKTSEKIPSSHTLMDGGSTKYWSGNSEFHFYSINVGGYFKLRSGEEISIEVSNPSLLDPDQDATYFGAFKVRDID

SEQ ID NO: 98 - protein, 171 aa, Artificial Sequence
  Human RANKL variant D1-3 monomer B His tag
MGSSHHHHHHSQDPEAQPFAHLTINATDIPSGSHKVSLSSWYHDRGWAKISNMTFSNGKLIVNQDGYYYLYANICFRHHETSGDLATEYLQLMVYVTKTSEKIPSSHTLMDGGSTKYWSGNSEFHFYSINVGGYFKLRSGEEISIEVSNPSLLDPDQDATYFGAFKVRDID

SEQ ID NO: 99 - protein, 158 aa, Artificial Sequence
  Human RANKL variant D2-1 monomer A
MEAQPFAHLVINATDIPSGSHKVSLSSWYHDRGWAKISNMTFSNGKLIVNQDGFYYLYANISFRHHETSGKLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 100 - protein, 171 aa, Artificial Sequence
  Human RANKL variant D2-1 monomer A His tag
MGSSHHHHHHSQDPEAQPFAHLVINATDIPSGSHKVSLSSWYHDRGWAKISNMTFSNGKLIVNQDGFYYLYANISFRHHETSGKLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 101 - protein, 158 aa, Artificial Sequence
  Human RANKL variant D2-1 monomer B
MEAQPFAHLVINATDIPSGSHKVSLSSWYHDRGWAKISNMTFSNGKLIVNQDGYYYLYANICFRHHETSGKLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 102 - protein, 171 aa, Artificial Sequence
  Human RANKL variant D2-1 monomer B His tag
MGSSHHHHHHSQDPEAQPFAHLVINATDIPSGSHKVSLSSWYHDRGWAKISNMTFSNGKLIVNQDGYYYLYANICFRHHETSGKLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 103 - protein, 158 aa, Artificial Sequence
  Human RANKL variant D2-2 monomer A
MEAQPFAHLVINATDIPSGSHKVSLSSWYHDRGWAKISNMTFSNGKLRVNQDGFYYLYANISFRHHETSGKLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 104 - protein, 171 aa, Artificial Sequence
  Human RANKL variant D2-2 monomer A His tag
MGSSHHHHHHSQDPEAQPFAHLVINATDIPSGSHKVSLSSWYHDRGWAKISNMTFSNGKLRVNQDGFYYLYANISFRHHETSGKLATEYLQLMVYVTKTSIKIPSSHTLMKGGSTKYWSGNSEFHYYSINVGGFFKLRSGEEISIEVSNPSLLDPDQDATYFGAFKVRDID

SEQ ID NO: 105 - protein, 158 aa, Artificial Sequence
  Human RANKL variant D2-2 monomer B
MEAQPFAHLVINATDIPSGSHKVSLSSWYHDRGWAKISNMTFSNGKLRVNQDGYYYLYANICFRHHETSGKLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 106 - protein, 171 aa, Artificial Sequence
  Human RANKL variant D2-2 monomer B His tag
MGSSHHHHHHSQDPEAQPFAHLVINATDIPSGSHKVSLSSWYHDRGWAKISNMTFSNGKLRVNQDGYYYLYANICFRHHETSGKLATEYLQLMVYVTKTSIKIPSSHTLMDGGSTKYWSGNSEFHFYSINVGGYFKLRSGEEISIEVSNPSLLDPDQDATYFGAFKVRDID

SEQ ID NO: 107 - protein, 158 aa, Artificial Sequence
  Human RANKL variant D2-3 monomer A
MEAQPFAHLVINATDIPSGSHKVSLSSWYHDRGWAKISNMTFSNGKLIVNQDGFYYLYANISFRHHETSGKLATEYLQLMVYVTKTSEKIPSSHTLMKGGSTKYWSGNSEFHYYSINVGGFFKLRSGEEISIEVSNPSLLDPDQDATYFGAFKVRDID

SEQ ID NO: 108 - protein, 171 aa, Artificial Sequence
  Human RANKL variant D2-3 monomer A His tag
MGSSHHHHHHSQDPEAQPFAHLVINATDIPSGSHKVSLSSWYHDRGWAKISNMTFSNGKLIVNQDGFYYLYANISFRHHETSGKLATEYLQLMVYVTKTSEKIPSSHTLMKGGSTKYWSGNSEFHYYSINVGGFFKLRSGEEISIEVSNPSLLDPDQDATYFGAFKVRDID

SEQ ID NO: 109 - protein, 158 aa, Artificial Sequence
  Human RANKL variant D2-3 monomer B
MEAQPFAHLVINATDIPSGSHKVSLSSWYHDRGWAKISNMTFSNGKLIVNQDGYYYLYANICFRHHETSGKLATEYLQLMVYVTKTSEKIPSSHTLMDGGSTKYWSGNSEFHFYSINVGGYFKLRSGEEISIEVSNPSLLDPDQDATYFGAFKVRDID

SEQ ID NO: 110 - protein, 171 aa, Artificial Sequence
  Human RANKL variant D2-3 monomer B His tag
MGSSHHHHHHSQDPEAQPFAHLVINATDIPSGSHKVSLSSWYHDRGWAKISNMTFSNGKLIVNQDGYYYLYANICFRHHETSGKLATEYLQLMVYVTKTSEKIPSSHTLMDGGSTKYWSGNSEFHFYSINVGGYFKLRSGEEISIEVSNPSLLDPDQDATYFGAFKVRDID

SEQ ID NO: 111 - DNA, 513 bp, Artificial Sequence
  Monomer D3a-1
atgggcagcagccatcaccatcatcaccacagccaggatccggaagcgcaaccgttcgcacacctgaccatcaatgctacggatattccttctggtagccataaagtctcgctgtccagctggtatcacgaccgtggttgggcagatattagcaatatgacgttttccaatggtaaactgattgtgaaccaagacggcttctactacctgtacgcgaacattagcttccgtcaccatgaaaccagcggcgacctggcgactgagtatctgcaactgatggtgtatgttacgaaaacgtcgatcaagattccgtccagccataccctgatgaaaggtggcagcaccaagtactggagcggcaacagcgagtttcactactatagcatcaacgtgggtggtttcttcaagctgcgtagcggtgaggaaatcagcattgaagtcagcaatccgagcctgctggacccggatcaggacgcaacttactttggtgcctttaaggttcgtgatattgac

SEQ ID NO: 112 - DNA, 474 bp, Artificial Sequence
  Monomer D3b-1
atggaagcccaaccatttgcacatttgacgattaacgccaccgacatcccgagcggtagccacaaggtgagcctgagctcttggtaccatgaccgcggctgggcgaaaatctctaatatgaccttcagcaacggcaagctgattgttaaccaggatggttactactacttgtatgctaacatttgctttcgtcaccacgaaacctccggcgatctggcgaccgagtatttgcagctgatggtttacgttaccaagacgagcatcaaaatcccgagctcccacaccctgatggatggtggtagcacgaagtattggagcggtaatagcgagttccacttctatagcattaatgtcggcggttactttaaactgcgctccggtgaggagatcagcatcgaggtgtctaatccgagcttgctggacccggaccaggatgcgacctattttggcgcgttcaaagtccgcgacatcgac

SEQ ID NO: 113 - DNA, 513 bp, Artificial Sequence
  Monomer D3a-2
atgggcagcagccatcaccatcatcaccacagccaggatccggaagcgcaaccgttcgcacacctgaccatcaatgctacggatattccttctggtagccataaagtctcgctgtccagctggtatcacgaccgtggttgggcagatattagcaatatgacgttttccaatggtaaactgcgtgtgaaccaagacggcttctactacctgtacgcgaacattagcttccgtcaccatgaaaccagcggcgacctggcgactgagtatctgcaactgatggtgtatgttacgaaaacgtcgatcaagattccgtccagccataccctgatgaaaggtggcagcaccaagtactggagcggcaacagcgagtttcactactatagcatcaacgtgggtggtttcttcaagctgcgtagcggtgaggaaatcagcattgaagtcagcaatccgagcctgctggacccggatcaggacgcaacttactttggtgcctttaaggttcgtgatattgac

SEQ ID NO: 114 - DNA, 474 bp, Artificial Sequence
  Monomer D3b-2
atggaagcccaaccatttgcacatttgacgattaacgccaccgacatcccgagcggtagccacaaggtgagcctgagctcttggtaccatgaccgcggctgggcgaaaatctctaatatgaccttcagcaacggcaagctgcgtgttaaccaggatggttactactacttgtatgctaacatttgctttcgtcaccacgaaacctccggcgatctggcgaccgagtatttgcagctgatggtttacgttaccaagacgagcatcaaaatcccgagctcccacaccctgatggatggtggtagcacgaagtattggagcggtaatagcgagttccacttctatagcattaatgtcggcggttactttaaactgcgctccggtgaggagatcagcatcgaggtgtctaatccgagcttgctggacccggaccaggatgcgacctattttggcgcgttcaaagtccgcgacatcgac

SEQ ID NO: 115 - DNA, 513 bp, Artificial Sequence
  Monomer D3a-3
atgggcagcagccatcaccatcatcaccacagccaggatccggaagcgcaaccgttcgcacacctgaccatcaatgctacggatattccttctggtagccataaagtctcgctgtccagctggtatcacgaccgtggttgggcagatattagcaatatgacgttttccaatggtaaactgattgtgaaccaagacggcttctactacctgtacgcgaacattagcttccgtcaccatgaaaccagcggcgacctggcgactgagtatctgcaactgatggtgtatgttacgaaaacgtcggagaagattccgtccagccataccctgatgaaaggtggcagcaccaagtactggagcggcaacagcgagtttcactactatagcatcaacgtgggtggtttcttcaagctgcgtagcggtgaggaaatcagcattgaagtcagcaatccgagcctgctggacccggatcaggacgcaacttactttggtgcctttaaggttcgtgatattgac

SEQ ID NO: 116 - skipped

SEQ ID NO: 117 - DNA, 474 bp, Artificial Sequence
  Monomer D3b-3
atggaagcccaaccatttgcacatttgacgattaacgccaccgacatcccgagcggtagccacaaggtgagcctgagctcttggtaccatgaccgcggctgggcgaaaatctctaatatgaccttcagcaacggcaagctgattgttaaccaggatggttactactacttgtatgctaacatttgctttcgtcaccacgaaacctccggcgatctggcgaccgagtatttgcagctgatggtttacgttaccaagacgagcgagaaaatcccgagctcccacaccctgatggatggtggtagcacgaagtattggagcggtaatagcgagttccacttctatagcattaatgtcggcggttactttaaactgcgctccggtgaggagatcagcatcgaggtgtctaatccgagcttgctggacccggaccaggatgcgacctattttggcgcgttcaaagtccgcgacatcgac

SEQ ID NO: 118 - DNA, 513 bp, Artificial Sequence
  Monomer d4a-1
atgggcagcagccatcaccatcatcaccacagccaggatccggaggcgcaaccgtttgcccacctggtgattaatgcgactgatatccctagcggtagccacaaagtttctctgtcgtcgtggtaccatgatcgcggttgggcgaagatttccaatatgaccttttccaatggtaaactgattgtgaaccaggacggtttttactacctgtacgcaaacatcagctttcgccaccacgaaaccagcggcaagctggccacggagtatctgcagctgatggtttatgtgaccaaaaccagcatcaaaatcccgtctagccacaccctgatgaaaggcggtagcacgaagtattggagcggtaactcggagtttcactactatagcattaatgtcggcggcttctttaaactgcgtagcggcgaggaaatctccattgaagtctccaacccgagcctgctggacccggaccaagatgcgacgtatttcggtgcgtttaaagtgcgtgacatcgac

SEQ ID NO: 119 - DNA, 474 bp, Artificial Sequence
  Monomer D4b-1
atggaggcccaaccgttcgcacatctggtaatcaacgcaaccgacatcccaagcggcagccacaaggtcagcctgagcagctggtatcatgatcgtggttgggctaagattagcaacatgaccttcagcaatggcaagttgattgtcaatcaggatggttattactatttgtatgctaatatctgcttccgtcaccatgaaacttccggtaagctggcgacggagtacctgcaactgatggtgtacgtgacgaaaacgagcatcaagattccgtccagccataccctgatggacggtggcagcaccaaatactggagcggcaacagcgagttccacttttactctattaacgttggtggctacttcaagttgcgcagcggtgaagaaatcagcattgaggttagcaatccgagcttgctggacccggatcaggacgcgacctacttcggtgcattcaaagttcgtgatattgac

SEQ ID NO: 120 - DNA, 513 bp, Artificial Sequence
  Monomer D4a-2
atgggcagcagccatcaccatcatcaccacagccaggatccggaggcgcaaccgtttgcccacctggtgattaatgcgactgatatccctagcggtagccacaaagtttctctgtcgtcgtggtaccatgatcgcggttgggcgaagatttccaatatgaccttttccaatggtaaactgcgtgtgaaccaggacggtttttactacctgtacgcaaacatcagctttcgccaccacgaaaccagcggcaagctggccacggagtatctgcagctgatggtttatgtgaccaaaaccagcatcaaaatcccgtctagccacaccctgatgaaaggcggtagcacgaagtattggagcggtaactcggagtttcactactatagcattaatgtcggcggcttctttaaactgcgtagcggcgaggaaatctccattgaagtctccaacccgagcctgctggacccggaccaagatgcgacgtatttcggtgcgtttaaagtgcgtgacatcgac

SEQ ID NO: 121 - DNA, 474 bp, Artificial Sequence
  Monomer D4b-2
atggaggcccaaccgttcgcacatctggtaatcaacgcaaccgacatcccaagcggcagccacaaggtcagcctgagcagctggtatcatgatcgtggttgggctaagattagcaacatgaccttcagcaatggcaagttgcgtgtcaatcaggatggttattactatttgtatgctaatatctgcttccgtcaccatgaaacttccggtaagctggcgacggagtacctgcaactgatggtgtacgtgacgaaaacgagcatcaagattccgtccagccataccctgatggacggtggcagcaccaaatactggagcggcaacagcgagttccacttttactctattaacgttggtggctacttcaagttgcgcagcggtgaagaaatcagcattgaggttagcaatccgagcttgctggacccggatcaggacgcgacctacttcggtgcattcaaagttcgtgatattgac

SEQ ID NO: 122 - DNA, 513 bp, Artificial Sequence
  Monomer D4a-3
atgggcagcagccatcaccatcatcaccacagccaggatccggaggcgcaaccgtttgcccacctggtgattaatgcgactgatatccctagcggtagccacaaagtttctctgtcgtcgtggtaccatgatcgcggttgggcgaagatttccaatatgaccttttccaatggtaaactgattgtgaaccaggacggtttttactacctgtacgcaaacatcagctttcgccaccacgaaaccagcggcaagctggccacggagtatctgcagctgatggtttatgtgaccaaaaccagcgagaaaatcccgtctagccacaccctgatgaaaggcggtagcacgaagtattggagcggtaactcggagtttcactactatagcattaatgtcggcggcttctttaaactgcgtagcggcgaggaaatctccattgaagtctccaacccgagcctgctggacccggaccaagatgcgacgtatttcggtgcgtttaaagtgcgtgacatcgac

SEQ ID NO: 123 - DNA, 474 bp, Artificial Sequence
  Monomer D4b-3
atggaggcccaaccgttcgcacatctggtaatcaacgcaaccgacatcccaagcggcagccacaaggtcagcctgagcagctggtatcatgatcgtggttgggctaagattagcaacatgaccttcagcaatggcaagttgattgtcaatcaggatggttattactatttgtatgctaatatctgcttccgtcaccatgaaacttccggtaagctggcgacggagtacctgcaactgatggtgtacgtgacgaaaacgagcgagaagattccgtccagccataccctgatggacggtggcagcaccaaatactggagcggcaacagcgagttccacttttactctattaacgttggtggctacttcaagttgcgcagcggtgaagaaatcagcattgaggttagcaatccgagcttgctggacccggatcaggacgcgacctacttcggtgcattcaaagttcgtgatattgac

SEQ ID NO: 124 - protein, 14 aa, Artificial Sequence
  His tag
MGSSHHHHHHSQDP

(19) United States
(12) Patent Application Publication
(10) Pub. No.: US 2014/0096274 A1
(43) Pub. Date: Apr. 3, 2014

(54) TNF Family Ligand Variants

(71) Applicants: Wilhelmus Johannes Quax, Antonius Deusinglaan I (NL); Vicente R. Tur, Barcelona (ES); Luis Serrano, Barcelona (ES); Albert Martinus Van Der Sloot, Barcelona (ES); Robbert H. Cool, Antonius Deusinglaan I (NL); Aart H.G. Van Assen, Antonius Deusinglaan I (NL)

(72) Inventors: Wilhelmus Johannes Quax, Antonius Deusinglaan I (NL); Vicente R. Tur, Barcelona (ES); Luis Serrano, Barcelona (ES); Albert Martinus Van Der Sloot, Barcelona (ES); Robbert H. Cool, Antonius Deusinglaan I (NL); Aart H.G. Van Assen, Antonius Deusinglaan I (NL)

(21) Appl. No.: 13/997,311

(22) PCT Filed: Dec. 23, 2011
(86) PCT No.: PCT/IB11/55937

§ 371 Date: Dec. 9, 2013

(30) Foreign Application Priority Data
Dec. 24, 2010 (EP) 10252230.7

(51) Int. Cl. C07K 14/525

(52) U.S. Cl. CPC C07K 14/525
USPC 800/13; 530/350; 536/23.5; 435/320.1; 435/252.33; 435/358; 435/369; 435/252.31; 435/348; 435/254.21; 514/1.1; 514/16.9; 514/16.6; 514/16.7; 514/19.3

(57) ABSTRACT

The present invention relates to variants of TNF family ligands which have been mutated at the ligand trimerisation interface so that they are not capable of assembling into trimers, and either assemble into dimers or remain as monomers. Such ligands bind to the TNF receptor but are unable to activate it, effectively functioning as competitive inhibitors. The invention also relates to nucleic acids encoding the variants of TNF family ligands, vectors and host cells comprising the nucleic acid and methods for the treatment of diseases associated with aberrant signalling through a TNF receptor.

### BACKGROUND

The Tumor Necrosis Factor ligand (TNF) family is a family of ligands which are involved in a wide range of biological activities, including cell proliferation and apoptosis. There is a complex balance between immunostimulatory and immunoregulatory functions within this family that ensures that an individual is capable of appropriate immune responses. Genetic polymorphisms or other mutations in the TNF ligand receptor family can result in deregulation of immune homeostasis, which is implicated in pathogenesis. For this reason, the TNF family represents a prime target for therapeutic intervention.

Each of the TNF family of ligands interacts with its cognate receptor(s) to trigger a number of signalling pathways that are important for immune tolerance, in addition to providing both protective and pathogenic effects on tissues (35,36,37). Examples of such proteins include ligands such as Receptor Activator of NF-Kappa Beta-Ligand (RANKL), TNF-related apoptosis-inducing ligand (TRAIL), B-Cell Activating Factor (BAFF), A Proliferation-inducing Ligand (APRIL), TNFalpha, CD30L, CD40L, FasL, Light, and Tumor necrosis factor-like Weak inducer of apoptosis (Tweak), which are implicated in disease conditions such as rheumatoid arthritis, autoimmune diabetes, systemic lupus erythematosus (SLE), Sjörgen's syndrome, experimental autoimmune encephalomyelitis (EAE), inflammatory bowel disease (IBD), autoimmune lymphoproliferative syndrome (ALPS), multiple sclerosis, and cancers such as breast cancer.

RANKL is particularly implicated in disorders associated with reduced bone density, such as osteoporosis, bone lesions due to rheumatoid arthritis (RA), Paget's disease and malignancy induced bone disease because signalling through RANKL results in increased production of bone-resorbing osteoclast cells. Binding of RANKL to its cognate receptor Receptor Activator of NF-Kappa Beta (RANK) expressed on osteoclast progenitor cells is crucial for the differentiation of these progenitor cells into mature osteoclasts, while binding of RANKL to RANK expressed on mature osteoclasts prevents these cells undergoing apoptosis and stimulates their adherence to bone cells (5). Increased signalling through RANKL and the production of an increased number of bone-resorbing osteoclasts disrupts the delicate homeostatic balance between osteoclasts and bone tissue producing osteoblasts, leading to disorders associated with reduced bone density, such as those mentioned above.

More recently, RANKL has been implicated in breast cancer. Here it is responsible for proliferative changes to the mammary epithelium which can lead to tumorigenesis (45,46). A soluble form of the RANKL extracellular domain is also produced by several types of tumour cells, including myeloma and metastatic breast cancer and by activated T lymphocytes in rheumatoid arthritis (5). RANKL has also been shown to promote the migration of RANK expressing tumor cells to bone tissue (48, 49, 50) and to control the development of progestin sensitive breast cancer (45, 46). The inventors therefore hypothesise that inhibiting the interaction of RANKL with its cognate receptor RANK could be used for the treatment of cancer and other diseases.

All TNF ligand family members consist of anti-parallel β-sheets, which self-trimerise into the homotrimeric active form of the ligand. Sequence homology is highest between the residues found at the trimer interface. Once formed, a ligand trimer binds three cognate receptors, with each receptor either binding in the groove between two adjacent ligands or binding directly to one of the ligands. (33,34).

As well as binding to their cognate receptors in order to promote signalling, TNF family ligands also bind to naturally occurring decoy receptors. These decoy receptors are either membrane bound or soluble pseudo-receptors, to which the TNF ligand can bind, without activating cell signalling. Whilst bound to the decoy receptor, the TNF ligand is sequestered away from the active receptor, and receptor-mediated signalling is inhibited.

Osteoprotegerin (OPG) is a soluble decoy receptor for RANKL. It is produced by osteoblasts and binds RANKL, thus preventing RANKL binding to and activating RANK, and resulting in a reduction of osteoclast activity. Likewise, DcR1 and DcR2 are decoy receptors for TRAIL, and DcR3 is a decoy receptor for FasL. These receptors sequester TRAIL or FasL, respectively, away from the active signalling receptors, effectively acting as endogenous competitive inhibitors.

Recently, a number of groups have begun studying the TNF ligand family in an attempt to manipulate aberrant signaling which is often associated with pathogenesis. With regard to inhibition of receptor signaling, much of this work has been modeled on the role of the decoy receptors, and has focused on increasing the sequestration of TNF ligands in order reduce receptor-mediated signaling.

One approach is to use the extracellular ligand binding domain of the ligand's endogenous receptor and fuse it with the Fc domain of IgG. These chimeric soluble receptors function as artificial decoy receptors and prevent the endogenous ligand binding to the endogenous receptor by sequestering it (51). One well known example is Etanercept (Enbrel®), a chimera between the extracellular ligand binding domain of TNFR2 (p75) and the Fc domain of IgG1 and which is clinically used in the treatment of RA and other immune diseases (39,40). A similar approach has also been used with the RANKL decoy receptor OPG (44).

An alternative approach requires the administration of a trimeric TNF ligand mutant incapable of receptor binding but capable of subunit exchange with the wild-type TNF ligand. This may result in the formation of mixed mutant/endogenous ligand trimers which are either incapable of receptor binding; as are the trimeric mutant forms, or, if bound, incapable of activating the receptor once bound (47). The trimeric wild-type ligand is essentially “poisoned” by the mutant variant and as a result, inactivated. A drawback of this approach is the heterogeneity of the preparation containing different ratios of wild-type to mutant monomers in the trimer, which makes the effects unpredictable.

Another strategy which has been successfully applied is the administration of anti-ligand antibodies (e.g Denosumab, also known as AMG 162 and Prolia®/Xgeva®) (42,43). These antibodies bind to RANKL and prevent it from binding to its cognate receptor, therefore blocking receptor signaling.

Recently, blocking peptides have also been used to block signaling through the RANK/RANKL pathway by binding directly to RANK and blocking the ligand binding site (31,32).

### DESCRIPTION OF THE INVENTION

The present invention relates to variants of TNF family ligands which have been mutated at the ligand trimerisation interface so that they are not capable of assembling into trimers, and either assemble into dimers or remain as monomers. Such ligands bind to the TNF receptor but are unable to activate it, effectively functioning as competitive inhibitors.

The invention also relates to nucleic acids encoding the variants of TNF family ligands, vectors and host cells comprising the nucleic acid and methods for the treatment of diseases associated with aberrant signalling through a TNF receptor.

Here, the inventors show that disrupting the trimerisation of TNF ligands prevents the formation of an active signaling complex. Furthermore, maintaining the ability of the ligand to bind to its cognate receptor, either in the form of a monomer or a dimer, blocks the receptor, competitively inhibiting signaling. TNF ligand variants which are not capable of trimerisation have the advantage that they are structurally similar to wild-type ligands, and are therefore less prone to proteolytic degradation than blocking peptides. The TNF ligand variants of the present invention therefore have potentially greater stability than blocking peptides due to a decreased rate of degradation and clearance from the body. Further, such a system exploits the TNF ligand signaling pathway in a manner distinct from pre-existing therapies as this approach targets the receptor and not the ligand and it can therefore be used alone or in combination with pre-existing therapies.

### Variants of TNF Family Ligands

In one aspect the present invention includes a variant of a TNF family ligand which is mutated such that it is not capable of assembling into a trimer, wherein the variant retains the ability to bind to one or more of its cognate receptor(s), but wherein binding to the receptor does not activate the receptor.

Within the context of the present invention, the term “mutated” encompasses substitution to any natural or non-natural amino acid residue, deletion, insertion and addition of amino acid residues.

In another embodiment the TNF ligand variant may include one or more post-translational modifications. Suitable modifications include pegylation, acetylation, formylation, alkylation such as methylation, and glycosylation, as well as labeling with fluorophores, radioisotopes, PET, etc. Such modifications may decrease immunogenicity, improve pharmacokinetics, and allow for certain specific applications of the variants (e.g. diagnostics).

As discussed above, TNF ligand trimerisation is required to effect receptor trimerisation, which is in turn required for signalling. Therefore, a variant of a TNF family ligand which is incapable of trimerisation, but able to assemble into a dimer or remain monomeric, and still able to bind its TNF receptor, will effectively block its receptor and therefore inhibit downstream signalling. The variant is therefore functioning as a competitive inhibitor.

Variants Capable of Assembling into Dimers

In one embodiment, the variant of a TNF family ligand may be capable of assembling into a dimer with another variant of the same TNF family ligand.

This will lead to formation of a TNF ligand dimer (homodimer or heterodimer), which is capable of binding to its cognate receptor, but will not be capable of activating the receptor because receptor trimerisation cannot occur when there is only a ligand dimer present.

In one embodiment the ligand variant has at least 10 fold, at least 100 fold, at least 1000 fold or more higher affinity for assembling into a dimer than assembling into a trimer.

In another embodiment, the ligand variant may have an affinity of at least 10⁸ M, at least 10⁹ M, at least 10¹⁰ M or greater for its cognate receptor(s). Affinity may be defined using the association constant (Kₐ), which is determined using the following formulae:

$$Ka = \frac{[ C ]}{[ R ] [ L ]}$$

Wherein [C] is the concentration of the complex, [R] is the concentration of unbound receptor and [L] is the concentration of unbound ligand.

In another embodiment, the ligand variant dissociation constant (K_{d}) of at least 10⁻⁸M, at least 10⁻⁹M, at least 10⁻¹⁰ M or less. Kd is determined using the following formula:

$$Kd = \frac{[ R ] [ L ]}{[ C ]}$$

Wherein [C] is the concentration of the complex, [R] is the concentration of unbound receptor and [L] is the concentration of unbound ligand.

In comparison to previous approaches, in particular the trimeric TNF ligand mutants discussed above, the present invention potentially ensures a faster response with a lesser dose, as it allows improvement of the Kd to provide stronger antagonists.

As discussed above, many TNF family receptors bind to their cognate ligands in the groove formed between two ligands of the trimer (e.g. RANK, TNFR1, TNFR2, FAS, CD40, CD27, CD30, DR4 and DR5). In this case a dimer will be required in order to stably bind, and therefore block, a single TNF receptor. Only one receptor will be bound by such a dimer due to the presence of only one cleft in which a receptor can be bound. The receptors will therefore be blocked, and no signalling will occur.

As also discussed above, some TNF family receptors bind their cognate ligands within the structure of the TNF-ligand monomer, generally the solvent exposed surface of the TNF ligand monomer (e.g. APRIL and BAFF). In this embodiment, the dimer is formed of two variant TNF ligands that bind to two TNF receptors. However, receptor activation will not occur due to the absence of the third ligand, which is required for receptor trimerisation.

In one embodiment, the TNF family ligand from which the variant is derived may be selected from the group consisting of RANKL (human accession no. AAB86811, mouse accession no. O35235), TRAIL (human accession no. P50591), APRIL (human accession no. BAE16556), BAFF (human accession no: Q9Y275), TNFalpha (human accession no. NP_{—}000585), TNFbeta (human accession no. P01374), CD30L (human accession no. NP_{—}001235; also NM_{—}001244), CD40L (human accession no. NP_{—}000065), FasL (human accession no. NP_{—}000630), Light (human accession no. O43557), Tweak (human accession no. BAE16557), GITRL (human accession no. AAQ89227), EDA-A1 (human accession no. Q92838), and OX40L (human accession no. P23510). Within this embodiment, the TNF family ligand may be of mammalian origin. More specifically, the TNF family ligand may be human, camel, dog, cat, horse, cow, pig, sheep, camelid, mouse, rat, rabbit, hamster, guinea pig, pig, sheep, and so on.

The variant of a TNF family ligand may be mutated at one or more positions. In certain embodiments, the variant may be mutated at 1, 2, 3, 4, 5, 6, 7, 8, 9, 10, 11, 12, 13, 14, 15, 16, 17, 18, 19, 20 or more positions.

It is preferred that the ligand variant is a soluble ligand variant. In this embodiment, the ligand may have been mutated to delete the transmembrane domain. For example, in the case of human RANKL, the ligand variant may comprise or consist of amino acid residues 159-317 or 161-317, and variants around this sequence. A RANKL ligand variant comprising or consisting of amino acids 161-317 is preferred. It will be clear to the skilled reader how to design equivalent soluble ligand variants from other TNF ligand family members and specific examples are given in more detail below.

### RANKL Variants

The inventors have illustrated the principle of preparing TNF ligand variants which are incapable of forming trimers using RANKL. In a preferred embodiment the TNF family ligand is RANKL.

The results described herein using RANKL can be equally applied to other TNF family ligands including TRAIL, APRIL, BAFF, TNFalpha, TNFbeta, CD30L, CD40L, FasL, Light, and Tweak, GITRL, EDA-A1, EDA-A2 and OX40L.

In one embodiment the RANKL variant may comprise or consist of a mutation at one or more of positions 169, 195, 213, 230, 257, 272, and 280 in the human RANKL sequence, for example, at 2 or 3 or 4 or 5 or 6 or all 7 of these positions (representative example combinations in human RANKL include 195 and 272; 213, 257 and 280; 213 and 280; 169, 230 and 272; 169, 213, 230, 257 and 280).

Included as aspects of the present invention are TNF ligand variants mutated at equivalent positions in orthologous proteins. For example, in the murine sequence, these positions are 168, 194, 212, 229, 256, 271, and 279. Mutations at such positions may be to any natural or non-natural amino acid, although mutations to more hydrophilic amino acids are preferred because residues positioned at the trimer interface will not be surface exposed following formation of a trimer, but at least some will be surface exposed for a TNF ligand variant incapable of forming a trimer.

In one embodiment a human RANKL variant may comprise or consists of one or more of the mutations T169V, K195D, F213Y, D230K, K257D, F272Y and F280Y. In the murine RANKL variant, these mutations are T168V, K194D, F212Y, D229K, K256D, F271Y and F279Y. Therefore, the variant may comprise or consist of 1, 2, 3, 4, 5, 6 or 7 of these mutations.

In one embodiment the variant may comprise a mutation at position 169 of the human RANKL sequence or at a position equivalent thereto in orthologous proteins. In the murine sequence, this position is 168.

In one embodiment the variant may comprise the mutation T169V in the human RANKL sequence or at a position equivalent thereto in orthologous proteins. In the murine sequence this mutation is T168V.

In one embodiment the variant may comprise a mutation at position 195 of the human RANKL sequence or at a position equivalent thereto in orthologous proteins. In the murine sequence, this position is 194.

In one embodiment the variant may comprise the mutation K195D in the human RANKL sequence. In the murine sequence this mutation is K194D.

In one embodiment the variant may comprise a mutation at position 213 of the human RANKL sequence or at a position equivalent thereto in orthologous proteins. In the murine sequence, this position is 212.

In one embodiment the variant may comprise the mutation F213Y in the human RANKL sequence or at a position equivalent thereto in orthologous proteins. In the murine sequence this mutation is F212Y.

In one embodiment the variant may comprise a mutation at position 230 of the human RANKL sequence or at a position equivalent thereto in orthologous proteins. In the murine sequence, this position is 229.

In one embodiment the variant may comprise the mutation D230K in the human RANKL sequence or at a position equivalent thereto in orthologous proteins. In the murine sequence this mutation is S229K.

In one embodiment the variant may comprise a mutation at position 257 of the human RANKL sequence or at a position equivalent thereto in orthologous proteins. In the murine sequence, this position is 256.

In one embodiment the variant may comprise the mutation K257D in the human RANKL sequence or at a position equivalent thereto in orthologous proteins. In the murine sequence this mutation is K256D.

In one embodiment the variant may comprise a mutation at position 272 of the human RANKL sequence or at a position equivalent thereto in orthologous proteins. In the murine sequence, this position is 271.

In one embodiment the variant may comprise the mutation F272Y in the human RANKL sequence or at a position equivalent thereto in orthologous proteins. In the murine sequence this mutation is F271Y.

In one embodiment the variant may comprise a mutation at position 280 of the human RANKL sequence or at a position equivalent thereto in orthologous proteins. In the murine sequence, this position is 279.

In one embodiment the variant may comprise the mutation F280Y in the human RANKL sequence or at a position equivalent thereto in orthologous proteins. In the murine sequence this mutation is F279Y.

The variant may also comprise one or more additional mutations which improve the solubility or stability of the dimer. Therefore, in one embodiment, the variant may comprise a mutation at one or more of positions 207, 221 or 247 in the human RANKL sequence, or at a position equivalent thereto in orthologous proteins. In the murine sequence these position are 206, 220 and 246. Mutations at these positions may be to any natural or non-natural amino acid, although mutation to a more hydrophilic amino acids is preferred to improve stability. In particular, the mutations I207R, C221S and I247E in the human RANKL sequence (R206R, C220S and I246E in the murine RANKL sequence) are preferred. The variant may comprise 1, 2 or 3 of these mutations.

In one embodiment the RANKL variant may comprise or consist of mutations at positions K195, C221 and F272 in the human RANKL sequence, or equivalent positions in orthologous proteins (in the mouse these are K194, C220 and F271).

In one embodiment the RANKL variant may comprise or consist of the mutations K195D, C221S and F272Y in the human RANKL sequence, or equivalent mutations at equivalent positions in orthologous proteins (in the mouse these are K194D, C220S and F271Y). This variant is herein referred to as hD1-1A and forms a monomer in the hD1-1 dimer, with monomer hD1-1B (the equivalent in the mouse is referred herein as monomer mD1A, and together with monomer mD1B, forms dimer mD1).

In another embodiment the RANKL variant may comprise or consist of mutations at positions F213, K257 and F280 in the human RANKL sequence, or equivalent positions in orthologous proteins (in the mouse these are F212, K256 and F279).

In another embodiment the RANKL variant may comprise or consist of the mutations F213Y, K257D and F280Y in the human RANKL sequence, or equivalent mutations at equivalent positions in orthologous proteins (in the mouse these are F212Y, K256D and F279Y). This variant is herein referred to as hD1-1B and forms one monomer in the hD1-1 dimer, with monomer hD1-1A (the equivalent in the mouse is referred herein as monomer mD1B, and together with monomer mD1A, forms dimer mD1).

In one embodiment the RANKL variant may comprise or consist of mutations at positions K195, I207, C221 and F272 in the human RANKL sequence, or equivalent positions in orthologous proteins (in the mouse these are K194, R206, C220 and F271).

In one embodiment the RANKL variant may comprise or consist of the mutations K195D, I207R, C221S and F272Y in the human RANKL sequence, or equivalent mutations at equivalent positions in orthologous proteins (in the mouse these are K194D, R206R, C220S and F271Y). This variant is herein referred to as hD1-2A and forms one monomer in the hD1-2 dimer, with monomer hD1-2B.

In another embodiment the RANKL variant may comprise or consist of mutations at positions I207, F213, K257 and F280 in the human RANKL sequence, or equivalent positions in orthologous proteins (in the mouse these are R206, F212, K256 and F279).

In another embodiment the RANKL variant may comprise or consist of the mutations I207R, F213Y, K257D and F280Y in the human RANKL sequence, or equivalent mutations at equivalent positions in orthologous proteins (in the mouse these are R206R, F212Y, K256D and F279Y). This variant is herein referred to as hD1-2B and forms one monomer in the hD1-2 dimer, with monomer hD1-2A.

In one embodiment the RANKL variant may comprise or consist of mutations at positions K195, C221, I247 and F272 in the human RANKL sequence, or equivalent positions in orthologous proteins (in the mouse these are K194, C220, 1246 and F271).

In one embodiment the RANKL variant may comprise or consist of the mutations K195D, C221S, I247E and F272Y in the human RANKL sequence, or equivalent mutations at equivalent positions in orthologous proteins (in the mouse these are K194D, C220S, I246E and F271Y). This variant is herein referred to as hD1-3A and forms one monomer in the hD1-3 dimer, with monomer hD1-3B.

In another embodiment the RANKL variant may comprise or consist of mutations at positions F213, I247, K257 and F280 in the human RANKL sequence, or equivalent positions in orthologous proteins (in the mouse these are F212, I246, K256 and F279).

In another embodiment the RANKL variant may comprise or consist of the mutations F213Y, I247E, K257D and F280Y in the human RANKL sequence, or equivalent mutations at equivalent positions in orthologous proteins (in the mouse these are F212Y, I246E, K256D and F279Y). This variant is herein referred to as hD1-3B and forms one monomer in the hD1-3 dimer, with monomer hD1-3A.

In another embodiment the RANKL variant may comprise or consist of mutations at positions T169, C221, D230 and F272 in the human RANKL sequence, or equivalent positions in orthologous proteins (in the mouse these are T168, C220, S229 and F271). In another embodiment the RANKL variant may comprise or consist of the mutations T169V, C221S, D230K and F272Y in the human RANKL sequence, or equivalent mutations at equivalent positions in orthologous proteins (in the mouse these are T168V, C220S, S229K and F271Y). This variant is known herein as hD2-1A and forms the hD2-1 dimer, together with monomer hD2-1B (the equivalent in the mouse is referred herein as monomer mD2A, and together with monomer mD2B, forms dimer mD2).

In another embodiment the RANKL variant may comprise or consist of mutations at positions T169, F213, D230, K257 and F280 in the human RANKL sequence or equivalent positions in orthologous proteins (in the mouse these are T168, F212, S229, K256 and F279).

In another embodiment the RANKL variant may comprise or consist of the mutations T169V, F213Y, D230K, K257D and F280Y in the human RANKL sequence or equivalent mutations at equivalent positions in orthologous proteins (in the mouse these are T168V, F212Y, S229K, K256D and F279Y). This monomer is known herein as hD2-1B, and forms the dimer hD2-1, together with monomer h2-1A (the equivalent in the mouse is referred herein as mD2B, and forms the dimer mD2, together with monomer mD2A).

In another embodiment the RANKL variant may comprise or consist of mutations at positions T169, I207, C221, D230 and F272 of the human RANKL sequence, or equivalent positions in orthologous proteins (in the mouse these are T168, R206, C220, S229 and F271).

In another embodiment the RANKL variant may comprise or consist of the mutations T169V, I207R, C221S, D230K and F272Y in the human RANKL sequence, or equivalent mutations at equivalent positions in orthologous proteins (in the mouse these are T168V, R206R, C220S, S229K and F271Y). This monomer is known herein as hD2-2A, and forms the dimer hD2-2, together with monomer hD2-2B.

In another embodiment the RANKL variant may comprise or consist of mutations at positions T169, I207, F213, D230, K257 and F280 in the human RANKL sequence or equivalent positions in orthologous proteins (in the mouse these are T168, R206, F212, S229, K256 and F279).

In another embodiment the RANKL variant may comprise or consist of the mutations T169V, I207R, F213Y, D230K, K257D and F280Y in the human RANKL sequence or equivalent mutations at equivalent positions in orthologous proteins (in the mouse these are T168V, R206, F212Y, S229K, K256D and F279Y). This monomer is known herein as hD2-2B, and forms the dimer hD2-2, together with monomer hD2-2A.

In another embodiment the RANKL variant may comprise or consist of mutations at positions T169, C221, D230, I247 and F272 in the human RANKL sequence, or equivalent positions in orthologous proteins (in the mouse these are T168, C220, S229, I246 and F271).

In another embodiment the RANKL variant may comprise or consist of the mutations T169V, C221S, D230K, I247E and F272Y in the human RANKL sequence, or equivalent mutations at equivalent positions in orthologous proteins (in the mouse these are T168V, C220S, S229K, I246E and F271Y). This monomer is known herein as hD2-3A, and forms the dimer hD2-3, together with monomer hD2-3B.

In another embodiment the RANKL variant may comprise or consist of mutations at positions T169, F213, D230, I247, K257 and F280 of the human RANKL sequence or equivalent positions in orthologous proteins (in the mouse these are T168, F212, S229, I246, K256 and F279).

In another embodiment the RANKL variant may comprise or consist of the mutations T169V, F213Y, D230K, I247E, K257D and F280Y in the human RANKL sequence or equivalent mutations at equivalent positions in orthologous proteins (in the mouse these are T168V, F212Y, S229K, I246E, K256D and F279Y). This monomer is known herein as hD2-3B, and forms the dimer hD2-3, together with monomer hD2-3A.

In another embodiment the RANKL variant may comprise or consist of mutations at positions C221 and F272 of the human RANKL sequence or equivalent positions in orthologous proteins (in the mouse these are C220 and F271).

In another embodiment the RANKL variant may comprise or consist of the mutations C221S and F272Y in the human RANKL sequence or equivalent mutations at equivalent positions in orthologous proteins (in the mouse these are C220S and F271Y). The mouse monomer is known herein as mD1′A, and forms the dimer mD1′, together with monomer mD1′B.

In another embodiment the RANKL variant may comprise or consist of mutations at positions F213, K257 and F280 of the human RANKL sequence or equivalent positions in orthologous proteins (in the mouse these are F212, K256 and F279).

In another embodiment the RANKL variant may comprise or consist of the mutations F213Y, K257D and F280Y in the human RANKL sequence or equivalent mutations at equivalent positions in orthologous proteins (in the mouse these are F212Y, K256D and F279Y). The mouse monomer is known herein as mD1′B, and forms the dimer mD1′, together with monomer mD1′A.

As described above, the present is not limited to RANKL, but can be extended to all members of the TNF family ligand. Therefore, the present invention includes a variant of a TNF ligand variant having mutations at positions equivalent to those discussed above in relation to RANKL. Such equivalent positions are detailed in Table 1, below. These positions are derived from FIGS. 31 and 32.

Human RANKL (hRANKL) has very high sequence similarity to mRANKL (>88% sequence identity, FIG. 22), and all but one amino acids at the positions recited above are conserved at least between mRANKL and hRANKL. Human counterparts to the mRANKL variants described herein are therefore highly similar in sequence to the mRANKL variants, and are included within the scope of the invention.

TABLE 1
Structurally equivalent mutations positions in other TNF family ligands
In one embodiment the invention includes the TNF ligand variants of SEQ ID NOs: 33-110.

| Mutations | | | | | | | | | | |
|---|---|---|---|---|---|---|---|---|---|---|
| position | Equivalent mutations positions in other TNF family ligands | | | | | | | | | |
| in murine | Human | Human | Human | Murine | Human | Human | Human | Human | Human | Human |
| RANKL | RANKL | TRAIL | BAFF | APRIL | TNFα | TNFβ | CD40L | APRIL | OX40L | GITRL |
| T168 | T169 | T127 | I150 | V112 | V93 | I68 | I127 | V121 | Q65 | G85 |
| K194 | K195 | F163 | F172 | L133 | L112 | F87 | T147 | L142 | — | — |
| F212 | F213 | F181 | Y192 | I153 | L131 | I106 | L168 | V162 | F100 | L116 |
| C220 | C221 | Y189 | L200 | L161 | L139 | V114 | T176 | L170 | Y108 | A124 |
| S229 | D230 | — | — | — | — | — | — | — | — | — |
| K256 | K257 | K224 | R231 | R186 | S171 | S150 | R207 | F194 | Q128 | T148 |
| F271 | F272 | L239 | N242 | Y199 | Y191 | L164 | Q220 | Y208 | — | — |
| F279 | F280 | I247 | I250 | V207 | V199 | A172 | V228 | V216 | V140 | T161 |

In other embodiments, the invention embraces any TNF superfamily member (including Human TRAIL, Human BAFF, human TNFα, human TNFβ, Human CD40L, Human APRIL, Human OX40L or Human GITRL) with a mutation at a position set out in the above table. Mutations at multiple positions are also aspects of the invention, so TNF ligand superfamily members may have 1, 2, 3, 4, 5, 6, 7, 8 or more mutations at the positions set out in the table below.

For completeness, it should be noted that the TNF family ligand variants may be synthesised with a tag sequence to aid purification. In case of a heterodimeric variant tag sequences can be fused to either one or both of the monomers comprising the heterodimer. The tag may be linked to either the N terminus or the C terminus of the protein, and may be linked either covalently or non-covalently. Preferably, the tag is linked covalently. This tag may be one of any number of suitable tags, as will be appreciated by one of skill in the art, such as a histidine tag, a FLAG tag, a biotin or streptavidin tag; preferably, the tag is a histidine tag, such as one with the sequence MGSSHHHHHHSQDP (SEQ ID NO: 124). The component monomers of the dimer may both be tagged, or just one may be tagged. If both are tagged, different tags may be used.

### Variant Dimers

In one aspect the invention includes a dimer comprising or consisting of two of the variants of TNF family ligands of the invention. Such a dimer may include any two TNF ligand variants of the same ligand described above. In a preferred embodiment the dimer comprises or consists of two of the RANKL variants discussed above.

In one embodiment, the dimer of two variants of TNF family ligands may be a heterodimer. The two variants included in the dimer may therefore contain different mutations to each other, relative to the wild-type sequence. Representative examples are the hD1-1, hD1-2, hD1-3, hD2-1, hD2-2 and hD2-3 dimers described above, which are preferred examples of dimers according to this aspect of the invention. Preferred dimer forms in the murine sequence are the mD1, mD1′ and mD2 dimers.

In one embodiment the two ligand variants may be present as a single peptide chain. Within this embodiment the sequences of the two ligand variants may be directly linked, or they may be linked through a linker sequence comprising or consisting of, for example, 1, 2, 3, 4, 5, 6, 7, 8, 9, 10, 15, 20, 30, 40, 50 or more amino acids.

In one embodiment the dimer may comprise or consist of a variant comprising or consisting of mutations at positions C221 and F272 of the human RANKL sequence or equivalent positions (mouse is C220 and F271), and a variant comprising or consisting of mutations at positions F213, K257 and F280 of the human RANKL sequence or equivalent positions (mouse is F212, K256 and F279).

In another embodiment the dimer may comprise or consist of a variant comprising or consisting of the mutations C221S and F272Y of the human RANKL sequence or equivalent mutations at equivalent positions (mouse is C220S and F271Y), and a variant comprising or consisting of the mutations F213Y, K257D and F280Y of the human RANKL sequence or equivalent mutations at equivalent positions (mouse is F212Y, K256D and F279Y). In the mouse, this is termed mD1′ herein.

In another embodiment the dimer may comprise or consist of a variant comprising or consisting of mutations at positions T169, C221, D230 and F272 of the human RANKL sequence or equivalent positions (mouse is T168, C220, S229 and F271) and a variant comprising or consisting of mutations at positions T169, F213, D230, K257 and F280 of the human RANKL sequence or equivalent positions (mouse is T168, F212, S229, K256 and F279).

In another embodiment the dimer may comprise or consist of a variant comprising or consisting of the mutations T169V, C221S, D230K and F272Y of the human RANKL sequence or equivalent mutations (mouse is T168V, C220A, S229K and F271Y) at equivalent positions and a variant comprising or consisting of the mutations T169V, F213Y, D230K, K257D and F280Y of the human RANKL sequence or equivalent mutations at equivalent positions (in mouse these are T168V, F212Y, S229K, K256D and F279Y). This dimer is herein termed hD2-1 in the human and mD2 in the mouse.

In one embodiment the TNF ligand variant dimer may comprise or consist of two of the variants of SEQ ID NOs: 35-110, with the proviso that the two variant monomers forming the dimer are variants of the same TNF ligand.

In a preferred embodiment, the two ligand variants are SEQ ID NOs: 35 and 36, SEQ ID NOs: 79 & 80, SEQ ID NOs: 81 & 82, SEQ ID NOs: 37 and 38, SEQ ID NOs: 83 & 84, SEQ ID NOs: 85 & 86, SEQ ID NOs: 87 & 88 SEQ ID NOs: 39 and 40, SEQ ID NOs: 89 & 90, SEQ ID NOs: 39 & 90, SEQ ID NOs: 89 & 40, SEQ ID NOs: 91 & 93, SEQ ID NOs: 92 & 94, SEQ ID NOs: 91 & 94, SEQ ID NOs: 92 & 93, SEQ ID NOs: 95 & 97, SEQ ID NOs: 96 & 98, SEQ ID NOs: 95 & 98, SEQ ID NOs: 96 & 97 SEQ ID NOs: 41 and 42, SEQ ID NOs: 99 & 101, SEQ ID NOs: 100 & 102, SEQ ID NOs: 99 & 102, SEQ ID NOs: 100 & 101, SEQ ID NOs: 103 & 105, SEQ ID NOs: 104 & 106, SEQ ID NOs: 103 & 106, SEQ ID NOs: 104 & 105, SEQ ID NOs: 107 & 109, SEQ ID NOs: 108 & 110, SEQ ID NOs: 107 & 110, or SEQ ID NOs: 108 & 109.

Variants Incapable of Assembling into Dimers

In one embodiment, the variant of a TNF family ligand may not be capable of assembling into a dimer with other variants of the same TNF ligand. In this embodiment, the variant will not be able to bind a receptor which binds within the cleft formed between two ligands because a ligand dimer will not form and there will therefore be no cleft into which a receptor can bind.

In one embodiment, the ligand variant binds its cognate receptor on the solvent exposed surface rather than in the cleft between two adjacent ligand monomers, as shown in FIG. 30. This will enable the ligand variant to bind to its cognate receptor without any requirement for the ligand variant to dimerise. Examples of TNF family ligands which bind in such a mode to their cognate receptor, and are therefore useful within this embodiment of the invention are APRIL, BAFF and most likely Tweak.

### Receptor Binding

As discussed above, TNF ligand variants of the invention bind to the TNF receptor but are unable to activate it.

The TNF ligand variants of the invention are considered to “bind to the TNF receptor” if they demonstrate more than 50% of the receptor binding observed with the wild-type ligand at saturating concentration. In other embodiments the ligand variants may demonstrate more than 60%, more than 70%, more than 80%, more than 90%, more than 91%, more than 92%, more than 93%, more than 94%, more than 95%, more than 96%, more than 97%, more than 98%, more than 99%, more than 99.5%, more than 99.6%, more than 99.7%, more than 99.8%, more than 99.9%, more than 99.95%, or more than 99.99% of the receptor binding observed with the wild-type ligand.

The TNF ligand variants of the invention are considered “unable to activate the receptor” if they demonstrate less than 50% of the receptor activation observed with the wild-type ligand at saturating concentration. In other embodiments the ligand variants may demonstrate less than 40%, less than 30%, less than 20%, less than 10%, less than 9%, less than 8%, less than 7%, less than 6%, less than 5%, less than 4%, less than 3%, less than 2%, less than 1%, less than 0.5%, less than 0.4%, less than 0.3%, less than 0.2%, less than 0.1%, less than 0.05%, or less than 0.01% of the receptor activation observed with the wild-type ligand.

In one embodiment, receptor activation may be assessed by measuring the levels of signalling through the receptor.

In one embodiment, the variant of a TNF family ligand according to the invention may have an increased binding affinity for one or more of its cognate receptor(s), compared to the wild-type TNF family ligand. The ligand variant may have increased binding affinity for 1, 2, 3, 4, 5 or more of its cognate receptors.

In one embodiment, the binding affinity for one or more of the cognate receptors may be increased by substituting one or more amino acids residues of the TNF family ligand to any other natural or non-natural amino acid, deleting one or more amino acid residues from the TNF family ligand or inserting one or more amino acid residues into the TNF family ligand.

Within this embodiment, binding affinity may be increased by substituting one or more of the amino acid residues present at the receptor binding interface of the ligand to any other natural or non-natural amino acid, or deleting any of the residues present at the receptor binding interface or inserting one or more additional residues at the receptor binding interface.

In another embodiment, the ligand variant may have an increased binding affinity for one or more of its cognate target receptors, compared to the wild-type TNF family ligand. In this context a “target” receptor refers to a receptor through which signalling can take place, i.e. not a decoy receptor, and which should be inhibited in order to produce a cellular response. Non-target receptors refer to active receptors which are not the main target of inhibition and/or decoy receptors.

Within this embodiment, affinity for the cognate target receptor may be increased by any of the methods discussed above.

In order to increase the binding affinity of a ligand variant for one or more of its cognate target receptors above its affinity for one or more of its decoy receptors, the ligand variant may include one or more amino acid residue substitutions or deletions which decrease the affinity of the ligand variant for one or more if its cognate decoy receptors.

In order to increase the binding affinity of a ligand variant for one or more of its cognate target receptors above its affinity for one or more of its non-target receptors, the ligand variant may include one or more amino acid residue substitutions, insertions or deletions which decrease the affinity of the ligand variant for one or more of its non-target cognate receptors. Such a receptor selective effect may be useful in cases where it is beneficial to inhibit the signalling mediated by one cognate receptor but not the signalling mediated by the other receptor(s) of the ligand. For example, in the treatment of Rheumatoid arthritis, it may be beneficial to block TNFalpha mediated signalling via TNFR1 but leave TNFalpha mediated TNFR2 signalling unperturbed. Such receptor selectivity is not possible, or at least not as readily possible, with the approaches used in the prior art.

### Sequence Identity, Fragments and Truncations

The TNF ligand variants of the invention may have at least 50% sequence identity to any one of the TNF ligands variants described herein and to SEQ ID NOs: 35-110. In one embodiment the variant may have at least 60%, at least 70%, at least 80%, at least 85%, at least 90%, at least 95%, at least 96%, at least 97%, at least 98%, at least 99%, at least 99.5%, at least 99.9% or more sequence identity to SEQ ID NOs: 35-110. The variant may retain the ability to bind its cognate receptor(s).

In one embodiment, the invention also encompasses fragments of the TNF ligand variants described herein and of SEQ ID NOs: 35-110. Such a fragment may be 7, 8, 9, 10, 20, 30, 40, 50, 60, 70, 80, 90, 100, 150, 200, 250, 300 or more amino acids in length. The variant fragment may retain the ability to bind its cognate receptor(s).

In another embodiment, the invention also encompasses TNF ligand variants as described herein which have a truncation at the N-terminus and/or the C-terminus. The truncation may be 1, 2, 3, 4, 5, 6, 7, 8, 9, 10, 11, 12, 13, 14, 15, 16, 17, 18, 19, 20, 30, 40, 50, 60, 70, 80, 90, 100, 150, 200 or more amino acids in length. The truncation may be an internal deletion with the same characteristics. The variant truncate may retain the ability to bind its cognate receptor(s).

As described above, in one preferred embodiment, the ligand variant may be a soluble ligand variant. Therefore, the ligand may have been mutated to delete the transmembrane domain.

The ligand variant may therefore comprise or consist of amino acid residues 158-316 or 160-316 of murine RANKL, and variants around this sequence, such as those described above.

The ligand variant may comprise or consist of amino acid residues 159-317 or 161-317 of human RANKL (161-317 is preferred), and variants around this sequence, such as those described above.

The ligand variant may therefore comprise or consist of amino acid residues 114-281 of human TRAIL, and variants around this sequence, such as those described above.

The ligand variant may therefore comprise or consist of amino acid residues 114-250 of human APRIL, and variants around this sequence, such as those described above.

The ligand variant may therefore comprise or consist of amino acid residues 134-285 of human BAFF, and variants around this sequence, such as those described above.

The ligand variant may therefore comprise or consist of amino acid residues 84-233 of human TNF alpha, and variants around this sequence, such as those described above.

The ligand variant may therefore comprise or consist of amino acid residues 62-205 of human TNF beta, and variants around this sequence, such as those described above.

The ligand variant may therefore comprise or consist of amino acid residues 95-234 of human CD30L, and variants around this sequence, such as those described above.

The ligand variant may therefore comprise or consist of amino acid residues 114-261 of human CD40L, and variants around this sequence, such as those described above.

The ligand variant may therefore comprise or consist of amino acid residues 130-281 of human FASL, and variants around this sequence, such as those described above.

The ligand variant may therefore comprise or consist of amino acid residues 83-240 of human LIGHT, and variants around this sequence, such as those described above.

The ligand variant may therefore comprise or consist of amino acid residues 94-249 of human TWEAK, and variants around this sequence, such as those described above.

The ligand variant may therefore comprise or consist of amino acid residues 72-199 of human GITRL, and variants around this sequence, such as those described above.

The ligand variant may therefore comprise or consist of amino acid residues 230-391 of human EDA-A1, and variants around this sequence, such as those described above.

The ligand variant may therefore comprise or consist of amino acid residues 51-183 of human OX40L, and variants around this sequence, such as those described above.

A table showing preferred sequences, extracellular forms, and preferred fragments, is set out below for TNF ligands according to the invention. As the skilled reader will appreciate, variants around these preferred fragments are included as aspects of the invention, which may be extended or truncated at either or both ends, for example, by 1, 2, 5, 10 or more amino acids.

| | | Listed | | full | | Soluble | | Preferred | |
|---|---|---|---|---|---|---|---|---|---|
| | | accession | UniProtkb/ | length | | extracellular | | fragment | |
| | species | no. | Swiss-Prot | begin | end | begin | end | begin | end |
| RANKL | human | AAB86811 | O14788 | 1 | 317 | 140 | 317 | 161 | 317 |
| RANKL | mouse | O35235 | O35235 | 1 | 316 | 139 | 316 | 160 | 316 |
| TRAIL | human | P50591 | P50591 | 1 | 281 | 95 | 281 | 114 | 281 |
| APRIL | human | BAE16556 | O75888 | 1 | 250 | 105 | 250 | 114 | 250 |
| BAFF | human | Q9Y275 | Q9Y275 | 1 | 285 | 134 | 285 | 134 | 285 |
| TNFa | human | NP_000585 | P01375 | 1 | 233 | 77 | 233 | 84 | 233 |
| TNFb | human | P01374 | P01374 | 1 | 205 | 62 | 205 | 62 | 205 |
| CD30L | human | NP_001235 | P32971 | 1 | 234 | 95 | 234 | 95 | 234 |
| CD40L | human | NP_000065 | P29965 | 1 | 261 | 113 | 261 | 114 | 261 |
| FASL | human | NP_000630 | P48023 | 1 | 281 | 130 | 281 | 130 | 281 |
| LIGHT | human | O43557 | O43557 | 1 | 240 | 83 | 240 | 83 | 240 |
| TWEAK | human | BAE16557 | O43508 | 1 | 249 | 94 | 249 | 94 | 249 |
| GITRL | human | AAQ89227 | Q9UNG2 | 1 | 199 | 72 | 199 | 72 | 199 |
| EDA- | human | Q92838 | Q92838 | 1 | 391 | 160 | 391 | 230 | 391 |
| A1 | | | | | | | | | |
| OX40L | human | P23510 | P23510 | 1 | 183 | 51 | 183 | 51 | 183 |

### Fusion Proteins

In one embodiment the ligand variants of the invention may be fused to a heterologous peptide. This may allow purification of the ligand variant during production, or may confer an additional therapeutic property to the variant for therapeutic or diagnostic use such as increasing retention time in the circulatory system by allow weak binding to abundant blood proteins such as serum albumin. A particularly suitable heterologous peptide for purification is a hexahistidine tag or a Flag tag.

For heterodimers comprising two differing ligand variants, the heterologous peptide maybe be fused to one of the monomers constituting the heterodimer or to both, alternatively two different heterologous peptide sequences may be fused to the two monomers constituting the heterodimer.

### Nucleic Acids

In one aspect, the invention includes a nucleotide sequence encoding a variant of a TNF family ligand according to the invention. The nucleic acid may encode any of the variants, truncates or fragments described above. The nucleic acid may be DNA including chromosomal DNA and cDNA or RNA, including mRNA. The mRNA or DNA sequences may or may not include one or more introns.

In one embodiment the nucleic acid may comprise or consist of any one of SEQ ID NOs: 111-123. Also included within the scope of the invention are fragments of these nucleic acid sequences. Such a fragment may be 7, 8, 9, 10, 20, 30, 40, 50, 60, 70, 80, 90, 100, 150, 200, 250, 300, 400, 500 or more nucleotides in length.

The nucleic acid may include one or more regulatory sequences such as a promoter, an enhancer, an internal ribosomal entry site (IRES), a Kozak sequence or a ribosomal binding site (RBS) sequence.

The monomers comprising the heterodimeric variants might be encoded in two separate nucleic acid molecules.

One or more ligand variants of the same TNF ligand may be encoded by a single nucleic acid molecule. In one embodiment the nucleic acid molecule may encode two ligand variants of the same ligand. In this embodiment expression of the two ligand variants may be under the control of the same or different promoters, IRES sites or other regulatory sequences.

In another aspect, the invention includes a vector comprising a nucleotide sequence encoding any of the variants, fragments or truncates of the invention. Vectors suitable for use in the method of the invention include plasmids and viruses (including both bacteriophage and eukaryotic viruses), as well as other linear or circular DNA carriers, such as those employing transposable elements or homologous recombination technology. Particularly suitable viral vectors include baculovirus-, lentivirus-, adenovirus- and vaccinia virus-based vectors.

In yet another aspect, the invention includes a host cell comprising a nucleic acid or a vector encoding one or more of the ligand variants of the invention. Suitable examples of host cells include prokaryotic host cells such as bacterial cells, yeast host cells and mammalian host cells. Particularly preferred host cells include E. coli, CHO, HEK293 PerC7 cells, B. subtilis, insect cells, and yeast such as S. cerevisiae.

### Receptor-Variant Complexes

The invention encompasses complexes which comprise or consist of one or more TNF ligand variants and one or more cognate TNF family receptors.

In one embodiment the complex may comprise or consist of a dimer formed of two TNF ligand variants which are incapable of assembling into a trimer and one or two cognate TNF family receptors.

In another embodiment the complex may comprise or consist of one or two TNF ligand variants which are incapable of assembling into dimers or trimers and one or two cognate TNF family receptors.

### Transgenic Animals

In another aspect, the invention includes a transgenic animal which has been engineered to express one or more of the TNF family variants or dimers disclosed herein.

The animal may be any animal which can be engineered to express one or more TNF ligand variants, for example a mammal. Examples of suitable animals include monkeys, mice, cows, sheep, rabbit, rat, hamster, guinea pig, goat, horses, donkey, pigs, cats, dogs and camels.

### Pharmaceutical Compositions

In one aspect the invention includes a pharmaceutical composition comprising one or more of the variants of a TNF family ligand, the dimers, the nucleotide sequences, the vectors or the host cells of the invention.

In one embodiment the pharmaceutical composition may additionally comprise a pharmaceutically-acceptable carrier, excipient, diluent or buffer. Suitable pharmaceutically acceptable carriers, excipients, diluents or buffers may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences (38).

The pharmaceutical composition may include a therapeutically effective amount of one or more of the variants of a TNF family ligand, the dimers, the nucleotide sequences, the vectors or the host cells of the invention. A pharmaceutically effective amount is an amount able to treat the disease. The actual amount will depend on a number of factors including the size, weight, age, gender, and health of an individual, and the rate of blood clearance, and will be decided by a clinical practitioner. Generally a pharmaceutically effective amount will be between 1 g/kg body weight and 1 mg/kg body weight or less.

In one embodiment the pharmaceutical composition may include an additional therapeutic component, in particular, a component useful for the treatment of osteoporosis, rheumatoid arthritis, Paget's disease or malignancy induced bone disease. Such a component may include Denosumab, antiresorptive agents, including bisphosphonates, the selective estrogen-receptor modulator (SERM), raloxifene, calcitonin, denosumab, teriparatide, calcium or vitamin D. In another embodiment the additional therapeutic component may be useful for the treatment of cancer, and may include chemotherapeutics, ERMs, SERMs, or specific biological targeted therapies e.g. Herceptin. In another embodiment the additional therapeutic component may be useful for the reducing inflammation any may include infliximab, etanercept, immunosupresives such as metroxate, and other anti-inflammatory agents such as NSAIDs.

The invention also includes any medical device which may have the pharmaceutical composition of the invention inserted into it or coated onto it. Such devices include, stents, pins, rods, meshes, beads, syringes, plasters, microchips, micro fluidic devices, and stitches.

### Methods of Treatment

In another aspect, the invention provides a variant of a TNF family ligand, a dimer, a nucleotide sequence, a vector of, a host cell or the pharmaceutical composition of the invention for use in medicine.

In one embodiment the invention includes a variant of a TNF family ligand, a dimer, a nucleotide sequence, a vector, a host cell or a pharmaceutical composition according to the invention for use in the treatment of osteoporosis, rheumatoid arthritis, Paget's disease, malignancy induced bone disease or cancer, such as breast cancer.

In another embodiment the invention includes a method of treating osteoporosis, rheumatoid arthritis, Paget's disease, malignancy induced bone disease or cancer such as breast cancer comprising administering a pharmaceutically effective amount of a variant of a TNF family ligand, a dimer, a nucleotide sequence, a vector of, a host cell or the pharmaceutical composition of the invention to a patient in need of treatment.

As used herein, the term “treatment” encompasses therapy, and can be prophylactic or therapeutic.

A pharmaceutically effective amount is an amount able to treat the disease. The actual amount will depend on a number of factors including the size, weight, age, gender, health of an individual, and the rate of blood clearance, and will be decided by a clinical practitioner. Generally a pharmaceutically effective amount will be between 1 g/kg body weight and 1 mg/kg body weight or less.

In another embodiment the invention includes the use of a variant of a TNF family ligand, a dimer, a nucleotide sequence, a vector, a host cell or a pharmaceutical composition according to the invention in the manufacture of a medicament for the treatment of osteoporosis, rheumatoid arthritis, Paget's disease, malignancy induced bone disease or cancer such as breast cancer.

The variant of a TNF family ligand, dimer, nucleotide sequence, vector, host cell or pharmaceutical composition according to the invention may be used for the treatment of disease in any animal. The animal may be a mammal such as a camel, dog, cat, horse, cow, pig, sheep, camelid, mouse, rat, rabbit, hamster, guinea pig, pig, sheep. In one embodiment, the mammal may be a human.

The variant of a TNF family ligand, dimer, nucleotide sequence, vector, host cell or pharmaceutical composition according to the invention may be administered to a patient using any one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

### Methods of Designing Variants of TNF Family Ligands

The invention includes a method for producing a variant of a TNF family ligand comprising the steps of:
a) identifying amino acids in the TNF family ligand that are located in the trimerisation interface as candidates for mutation;
b) substituting each of one or more residues in the trimerisation interface; and
c) selecting amino acid substitutions which have a neutral or positive effect on the stability of the dimer but a negative effect on the stability of the trimer.

In one embodiment, the most hydrophilic amino acid substitution at newly solvent exposed positions may be favoured.

In another embodiment the method may further comprise one or more of the following steps:
d) selecting amino acid substitutions to increase the affinity for one or more of the target receptor(s);
e) selecting amino acid substitutions to increase selectivity for one or more of the target receptors, either by increasing affinity for one or more of the target receptor(s) or decreasing affinity for one or more of the non-target receptors, including decoy receptors.
f) producing a variant of a TNF family ligand; and
g) Modifying the variant to improve its properties such as to decrease its immunogenicity or improve its pharmacokinetics. Such modifications may include one or more of pegylation, acetylation, formylation, alkylation such as methylation, and glycosylation and other possible modifications.

In one embodiment, the method may be a method of producing an inhibitory variant of a TNF family ligand.

### BRIEF DESCRIPTION OF THE FIGURES

FIG. 1 shows a schematic view of RANK inhibitor design. A) Top view of the RANKL-RANK signaling complex. Depicted is the native biological active signaling complex comprising of a RANKL homo trimer-individual monomers in green (monomer ‘A’), blue (monomer ‘B’) and yellow (monomer ‘C’) and three RANK receptors (red), with each individual receptor binding in the cleft between two RANKL monomers. Upon changing trimeric RANKL (left) into a dimeric variant (right) only one intact receptor binding interface is present. While still able to bind to RANK it is unable to form the biological active RANKL-RANK signaling complex. B) and C) Side view of the RANKL hetero dimeric inhibitory variants D1 (mD1) (B) and D2 (mD2) (C). The view is toward the former trimeric interface by rotating the structure in panel B 90° counter clock wise on its x-axis, the positions of amino acid substitutions used to construct the dimeric variants are depicted in red and the two monomers comprising each heterodimer—“A” and “B”—are shown in green and blue, respectively. D) all mutated positions in mRANKL used to construct the heterodimers mD1 and mD2 depicted in a single mRANKL monomer (side view orientation). E) and F) similar to FIGS. 1B and 1C but now depicting the amino acid substitutions used to construct the dimeric variants mD1 (E) and mD2 ((F).

FIG. 2 is a schematic view depicting the binding of a TNF family ligand trimer to 3 receptors (left), and the binding of a TNF family ligand dimer to a single receptor at the only cleft formed between two ligand monomers (right). The orientation of the ligand receptor complex is similar to FIG. 1a. The receptors are depicted as blue circles and the TNF ligand monomers as red, blue and green ribbons.

FIG. 3 shows capillary electrophoresis of A) murine wild-type RANKL, B) dimer 1 (mD1), and C) dimer 2 (mD2).

FIG. 4 shows biophysical characterization of the murine RANKL dimers. A) Capillary electrophoresis under denaturating conditions. The electropherograms of murine flagRANKL WT (flag-mRANKL WT) (upper trace) and mD2 (lower trace) are shown. The boxed area encloses the main protein peak. B) Detailed view of the boxed area of the main protein peak. C) Size exclusion chromatography. The upper trace shows the elution profile of flag-mRANKL WT (elution volume Ve: 10 ml) and the lower traces show the superimposed elution profiles of mD1, mD2 (Ve: 11 ml) and an mD2 version containing an additional flag-tag (Ve: 10.9 ml). The vertical line depicts the elution volume of flag-mRANKL WT and the tick marks indicate the elution volumes of calibration standards (C: Conalbumin, 75 kDa; O: Ovalbumin, 43 kDa; CA: Carbonic Anhydrase 29 kDa; R: Ribonuclease 13.7 kDa and A: Aprotinin, 6.5 kDa) and of the void volume (V₀ 8 ml) of the column. D) Apparent molecular weight as calculated by dynamic light scattering (DLS). WT is flag-mRANKL WT; D1 is mD1 and D2 is mD2.

FIG. 5 shows circular dichroism (CD) far UV wavelength spectrum and thermal denaturation. Upper panel) CD far UV wavelength spectrum (320-200 nm) of flag-mRANKL WT (WT), mD1 (D1) and mD2 (D2). Lower panel) Thermal denaturation as monitored by CD of flag-mRANKL WT, mD1 and mD2. Samples were heated from 25-90° C. with a rate of 40° C./hr, unfolding was measured at 222 nm. The CD signal is expressed in mean residue molar ellipticity units.

FIG. 6 shows a surface Plasmon resonance binding curve of A) murine RANKL WT and B) RANKL mD1 and C) RANKL mD2 binding to immobilized hRANK-FC receptor

FIG. 7 shows the results of a competitive ELISA using RANKL dimer mD2.

FIG. 8 shows osteoclastogenesis determination by TRAP (tartrate resistant acid posphatase) assay using A) 100 ng/ml wild-type mRANKL, B) 100 ng/ml RANKL dimer 2 (mD2), C) 100 ng/ml wild-type mRANKL and 100 ng/ml RANKL dimer 2 (mD2), and D) untreated.

FIG. 9 is a graphical representation of the results of FIG. 8, A) mD1+/−mRANKL WT and B) mD2+/−mRANKL WT.

FIG. 10 shows A) Molecular weight determination of flag-mRANKL WT, mD1 and mD2 by size exclusion chromatography using known calibration standards. Plotted is Kₐᵥ of sample or standard versus the logarithm of the molecular weight (Mr), with Kₐᵥ defined as the ratio between (elution volume of protein−column void volume) and (column volume−column void volume). The Mr of flag-mRANKL WT, mD1 and mD2 were obtained by interpolation of the individual Kₐᵥ's and the calibration line. The following calibration standards were used Conalbumin, 75 kDa; Ovalbumin, 43 kDa; Carbonic Anhydrase 29 kDa; Ribonuclease 13.7 kDa and Aprotinin, 6.5 kDa. B) Blue Native PAGE analysis. mD1 and mD2, and mRANKL WT were separated on a 12% Blue Native PAGE gel. The dimeric variants show a band below the 66 kDa BSA marker band while mRANKL WT shows a band above the 66 kDa BSA band.

FIG. 11 shows a circular dichroism (CD) wavelength scan of A) mD1, and B) mD2. Additional CD wavelength scan data with WT control and additional replicates of mD1 and mD2 can be found in the top panel of FIG. 5.

FIG. 12 shows a circular dichroism (CD) temperature scan of A) mD1, and B) mD2. Additional CD temperature scan data with WT control and additional replicates of mD1 and mD2 can be found in the lower panel of FIG. 5.

FIG. 13 shows analytical gel filtration chromatography for A) murine flag wild-type RANKL, B) RANKL mD2, and C) mD2 containing an additional N-terminal flagtag. Additional gel filtration chromatography data containing mD1 can be found in FIG. 4 C and FIG. 10 A.

FIG. 14 shows analytical gel filtration chromatography, where F is flag wild-type RANKL, E and C are RANKL mD2 and D is mD2 containing an additional N-terminal flagtag. FIG. 14 is based on FIG. 13 but having all elution profiles overlaid in a single figure. Additional gel filtration chromatography data containing mD1 can be found in FIG. 4 C and FIG. 10 A.

FIG. 15 shows kinetics of RANK binding as measured by SPR. The SPR sensorgrams show binding of murine RANKL WT, mD1 or mD2 to mRANK-Fc at a low capture density (A) or a high capture density (B). Sensorgrams in A represent measurements that were performed using a very low density of mRANK-Fc (7.5, 45 and 38 RU for WT, mD1 and mD2, respectively) to ensure a 1:1 interaction between ligand and receptor (see text). Measurements in B were performed at a high mRANK-Fc density (1880, 1942 and 1935 RU for WT, mD1 and mD2 respectively). Ligand was injected from 0-180 seconds and dissociation was monitored for an additional 470 s. (C) Sensorgrams used for the high density measurement as depicted in table 5. Arrows indicated the approximate response level in case of a 1:1, 1:2 or 1:3 interaction stoichiometry.

FIG. 16. Binding of murine RANKL dimers towards human RANK. Binding of 0.3 to 15 nM of murine RANKL WT, mD1 or mD2 towards A) mRANK-Fc or B) hRANK-Fc as measured by SPR using a single cycle kinetics method. Ligand was injected with 5 consecutive injections from the lowest to the highest concentration and after the last injection dissociation was monitored for 600 s. Binding data was fitted using a 1:1 interaction model. WT is flag-mRANKL WT, D1 is mD1 and D2 is mD2.

FIG. 17 shows ELISA of murine RANKL WT, mD1 and mD2 binding to immobilized (A) mRANK-Fc receptor or (B) murine OPG-Fc.

FIG. 18 shows A) Competitive inhibition of murine flagRANKL WT binding mRANK-Fc by RANKL mD1 and mD2. Binding of 2 nM of murine flagRANKL WT to immobilized mRANK-Fc in the presence of 0-400 nM mD1 or mD2 is measured by an anti-flag antibody and expressed as % binding relative to the binding of flag-mRANKL WT with no inhibitor present. The results are expressed as mean values+/−S.E.M. (n=3). B) EC50 shift of flag-mRANKL WT by mD2. Binding of 0-50 nM flag-mRANKL WT to immobilized mRANK-Fc in the presence or absence of various concentrations of mD2 (3, 5, 11, 22 or 43 nM). Binding of flag-mRANKL WT is measured using an anti-flag antibody. Results are expressed as mean+/−S.E.M. (n=3). D1 is mD1 and D2 is mD2. FIG. 18B is a duplicate of FIG. 7.

FIG. 19 shows A) RANKL WT-mediated osteoclastogenesis. Formation of multinucleated cells as observed by TRAP staining in murine RAW 264.7 cells upon treatment with 100 ng/mL mRANKL WT (WT); untreated cells (NT). Multinucleated cells are marked with arrows. B & C) Inhibition of RANKL-mediated osteoclastogenesis by mD1 and mD2. The relative number of multinucleated RAW 264.7 cells upon treatment with mRANKL WT alone or in the presence of 50 ng/mL and 100 ng/mL mD1 (B) or mD2 (C) as measured with the TRAP assay is shown. The results are expressed as mean values+/−S.E.M. (n=3). * indicates a statistically significant difference (p<0.05).

FIG. 20 shows the conservation between human and murine RANKL. 143 of the 149 residues (89%) are identical.

FIG. 21A shows the sequence conservation between the human and murine RANKL sequences of dimer 1 (mD1). Mutations in dimer A are shown in dark blue, mutations in dimer B are shown in light cyan, and mutations shared between dimers A and B are shown in dark green.

FIG. 21B shows the sequence conservation between the human and murine RANKL sequences of dimer 2 (mD2)_. Mutations in dimer A are shown in dark blue, mutations in dimer B are shown in light cyan, and mutations shared between dimers A and B are shown in dark green.

FIG. 22 shows sequence alignment of the extracellular domain of murine RANKL and human RANKL. Conserved positions are depicted as dots, amino acids that differ between the murine and human the sequence are indicated in red. Positions that are mutated in mD1 (A) and mD2 (B) are boxed and the substituted residues are shown above the alignment. This figure is a more concise figure based on FIGS. 21a and b.

FIG. 23 shows the non-conserved residue positions (red) between the human and murine RANKL sequences mapped on the dimer structure. A) shows the conserved trimer interface between the human and murine RANKL sequences as mapped on the dimer structure and B) shows the conserved receptor binding interface between the human and murine RANKL sequences as mapped on the dimer structure. The position of the receptor binding interface between the two adjacent monomer is indicated by the blue arrow.

FIG. 24 shows conformation of stoichiometry by Native Gel for Dimer 1 (mD1) and 2 (mD2). Making use of a NativePAGE™ Novex® 4-16% Bis-Tris Gel samples were loaded and the size was checked. Visible is one band at the expected size and two bands higher, the sizes do not correspond to the size of a trimer or monomer. A more complete version from a subsequent experiment containing WT control can be found in FIG. 10C.

FIG. 25 shows conformation of stoichiometry by DLS for mD1 and mD2. A) % Intensity plotted versus the radius measured for dimer 1. The first peak has a polydispersity of 35% for a measured radius of 2.8 nm corresponding to a 38 kDa protein, the second and third peak correspond to a radius which is to big to be a protein. B) % Intensity plotted versus the radius measured for dimer 2. The first peak has a polydispersity of <0.1% for a measured radius of 3.0 nm corresponding to a 46 kDa protein, the second and third peak correspond to a radius which is too large to be a protein. A more complete version from a subsequent experiment containing WT control can be found in FIG. 4D.

FIG. 26 shows representative curves of receptor binding to hRANK-Fc of wt-mRANK-L trimer and RANK-L Dimer 1 (mD1) and Dimer 2 (mD2) as determined with SPR. Concentration dependent association was reached for both dimers and for wildtype. The maximum response units are as expected for the interaction between dimeric RANK-L and one monomeric unit of mRANK-FC, as can be calculated with the formula {[MWbound/MWcaptured]×RU RANK-FCcaptured} which gives the expected maximum RU value. In the experiment with RANK-L Dimer 1 (mD1) 57 RU of mRANK-FC was captured, the expected maximum RU value would be 34. For the experiment with RANK-L Dimer 2 (mD2) 25 RU mRANK-FC was captured, the expected RU value would be 15. It is visible that both values are almost reached. For the trimeric mRANK-L 37RU mRANK-FC was captured, the maximum RU value expected here would be (105/60*37=) 65 RU. This value is the calculated maximum reached in the experiment shown. (A) mD1 (B) mD2 and (C) mWT. Please note, this figure shows magnified versions of the curves shown in FIG. 6.

FIG. 27 shows binding of mRANKL WT, mD1 and mD2 to hOPG as shown by SPR. Directly immobilized OPG on a CM5 chip, concentration range RANKL (4 nM-31.25 pM). Concentration dependent binding, almost no dissociation. The curves show the association phase, followed by the dissociation phase, regeneration of the bound ligand from the receptor and return to baseline.

FIG. 28 shows the results of an ELISA to measure the binding of mRANKL-WT, D1 (mD1) and D2 (mD2) to A) human RANK-Fc and B) human OPG-Fc.

FIG. 29 shows dimer 1 (mD1) treated cells (concentration 100 ng/mL), showing the formation of bigger osteoclasts.

FIG. 30 shows the association of wild-type TNF ligands with their cognate receptor, when ligands bind either A) in the cleft formed between two receptors, or B) to the surface exposed surface of the receptor, wherein the large blue ovals represent receptor monomers and the small red ovals represent ligand monomers.

FIG. 31 shows an alignment of the extracellular ligand binding domain of various TNF ligands against murine RANKL. Structurally equivalent positions of mRANKL mutations are shown in bold and underlined. All sequences shown are based on the X-ray structures, except human APRIL which is based on a homology model with murine April as template.

FIG. 32 shows a structural alignment of the extracellular ligand binding domain of various TNF ligands against murine RANKL. All sequences shown are based on the X-ray structures, except human APRIL which is based on a homology model with murine April as template. mRANKL is in black and the amino acid positions used to create the mRANKL dimers are indicated. In grey are the other TNF-ligands.

FIG. 33. Soluble expression of human dimeric RANKL variants in E. coli BLR (DE3) upon induction with IPTG. Cells were grown in LB at 37° C. while shaking at 220 rpm, upon reaching an OD600 protein expression was induced with 1 mM IPTG and temperature was lowered to 30° C. Samples were recovered 4 and 16 hrs (overnight) post induction. Proteins were extracted using Novagen Bugbuster according to the suppliers specifications and insoluble and soluble fraction were separated by centrifugation for 30 min at 20.000 g. Proteins in the soluble fraction were separated on a 4-12% NuPAGE gel (Invitrogen) and transferred by Western blot. Subsequently, histag containing proteins were detected using an anti-histag antibody (Sigma). Soluble expression could be demonstrated for all variants. From each variant 3 independent clones were tested. Of note, the signal from hRANKL D2.3 gave such a strong signal that it became overexposed.

FIG. 34 A). Purity of purified human RANKL dimer variants hD1-3 and hD2-3 as determined by SDS-PAGE/CBB stain. Proteins were purified using an Histrap column purification (HIS-TRAP: GE Lifesciences) step followed by a Gelfiltration step (Hiload 16/60 Superdex 75, GE Lifesciences). B) Difference in molecular weight as determined by a difference in elution volume between trimeric murine flagtag RANKL WT and human dimeric histag RANKL variant hD2-3 as measured on a Hiload 16/60 superdex 75 column (GE Lifesciences). Variant hD2-3 elutes at a later volume than trimeric flagtag RANKL WT and similar to murine dimeric RANKL mD1 and mD2.

FIG. 35. Binding of dimeric human RANKL variants and human RANKL WT towards murine RANK at 37° C. (left) or human RANK (right) receptor. Receptors were captured at a level of 200 RU using a Protein A (Sigma) modified surface of a CM5 chip (Biacore GE Lifesciences). Receptor binding curves of purified human hD1.3 and hD2.3 variants and human RANKL WT (E. coli produced, R&D systems) were recorded in real time using the single cycle kinetics method (Biacore) using a Biacore T100 (Biacore, GE Lifescience). Ligands were injected in 5 consecutive injection from 1 nM to 100 nM. After subtracting the signal from the reference channel and buffer injections the resulting binding curves were fitted to a 1:1 interaction model using the BiaEvaluation software (Biacore GE Lifescience). Kinetic constants and affinity constants are shown in table 11. Flow rate was 50 ul/min and protein A surface was regenerated using 10 mM Glycine pH 2 solution, murine RANK-Fc and human RANK-Fc were obtained from R&D systems. Running buffer was HBS-EP+ (Biacore GE Lifescience).

Various aspects and embodiments of the present invention will now be described in more detail by way of example. It will be appreciated that modification of detail may be made without departing from the scope of the invention.

### EXAMPLES

### Example 1

### In Silico Design of RANKL Dimers

RANKL, like most other TNF ligand family members, binds the RANK receptor in the cleft between two adjacent monomers and RANKL induced trimerization of the RANK receptor results in activation of downstream signalling. We reasoned that by engineering a dimeric RANKL variant having only one intact receptor binding interface a competitive antagonist could be created that binds RANK receptor but is unable to trimerize and activate it.

Several crystal structures are available of the extracellular domain of murine RANKL are available (20) but none of human RANKL. The two highest resolution structures of murine RANKL (PDB codes: 1IQA and 1S55; 2.20 and 1.90 Å respectively) were used for the design (20). Both structures contain the biological active RANKL homotrimer in the asymmetric unit comprising of three RANKL monomer chains (A, B and C). The strategy involved engineering mutations that will prevent trimer formation, while retaining one ligand monomer-monomer interface and the receptor binding interface, and prevent aggregation of the newly exposed surface. A dimeric structure was constructed by deleting monomer C, thus leaving newly solvent exposed patches on each of the two remaining monomers.

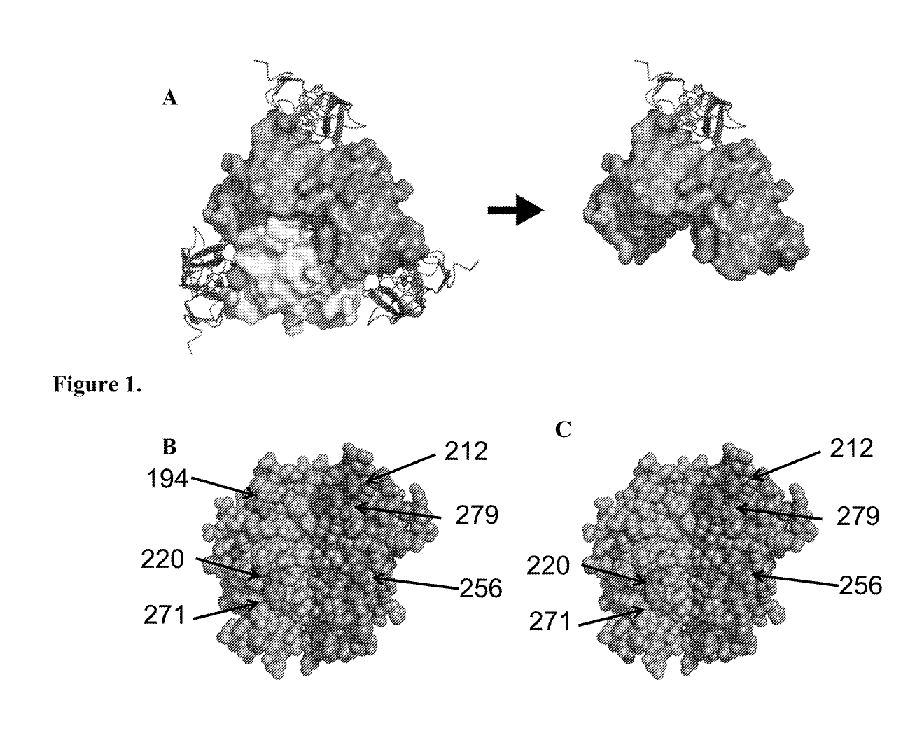

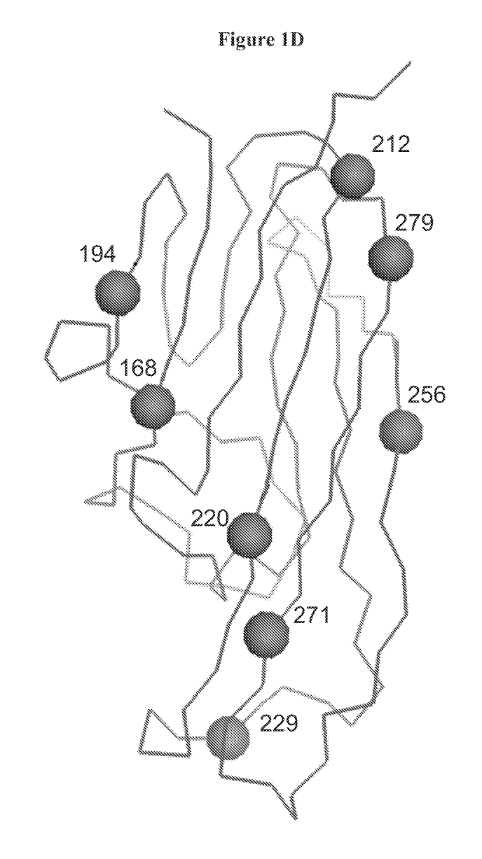

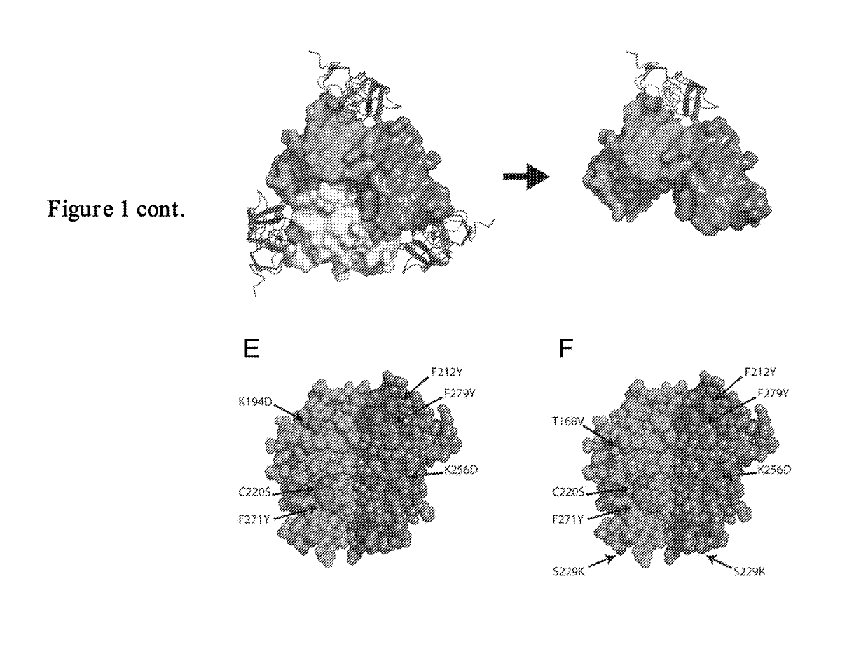

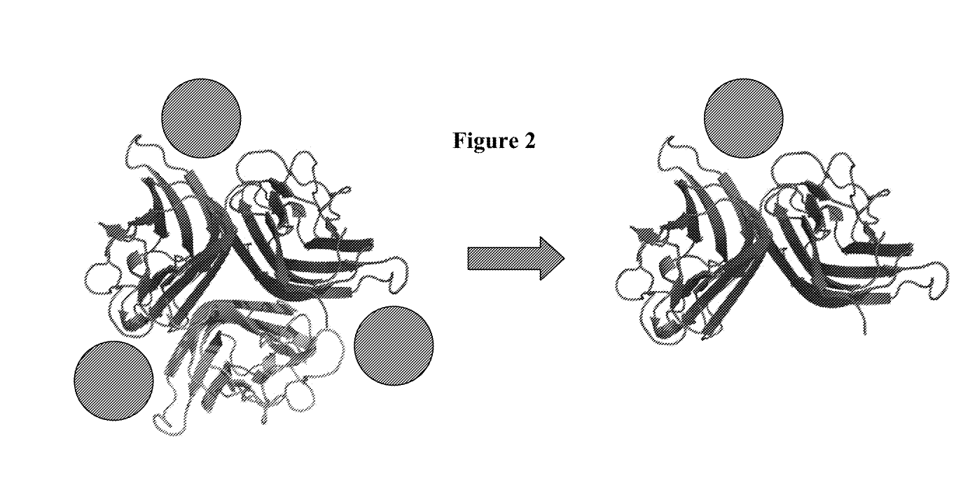

Next, an in silico amino acid mutagenesis scan using the FoldX protein design algorithm was performed on amino acid residues comprising the newly created solvent exposed interface of the dimer (FIG. 1) (25). Here, amino acid residues composing this newly created solvent exposed surface of the dimer model were mutated in silico to all other nineteen naturally occurring amino acids and the effect on stability of the dimeric structure and the trimeric structure was calculated by the FoldX protein design algorithm. The FoldX in silico mutagenesis and energy calculation were performed as described before. (25, 24, 15) Amino acids substitutions that had a neutral or positive effect on the stability of the dimer but a negative effect on the stability of the trimeric structure were retained. Favourable substitutions were combined and when possible the most hydrophilic amino acid substitution at newly solvent exposed positions was favoured. Remaining hydrophobic patches were modified by introducing energetically favourable hydrophilic substitutions. Favourable combinations of single amino acid substitutions were determined.

Two different designs: mD1 (mouse Dimer 1) and mD2 (mouse Dimer 2), each comprising of a monomer A and B, were proposed for experimental characterization. The mutations made in these dimers are shown in Table 2, below.

TABLE 2
Murine dimeric RANKL Variants.

| | Monomer | amino acid substitutions | | | | |
|---|---|---|---|---|---|---|
| Dimer 1 | | | | | | |
| variants | | | | | | |
| mD1 | A | K194D | C220S | F271Y | | |
| | B | F212Y | K256D | F279Y | | |
| mD1′ | A | C220S | F271Y | | | |
| | B | F212Y | K256D | F279Y | | |
| Dimer 2 | | | | | | |
| variant | | | | | | |
| mD2 | A | T168V | C220S | S229K | F271Y | |
| | B | T168V | F212Y | S229K | K256D | F279Y |

Due to the three fold symmetry axis of the RANKL trimer, construction of a heterodimer consisting of a monomer A and B, was required. For example, residue 271 in monomer A is in the interface that previously interacted with the third monomer and substituting wild-type Phe271 for a tyrosine residue reduces the affinity for the third monomer. On the other hand, in monomer B this residue is part of the interface that interacts with monomer A and here the F271Y substitution would severely affect the stability of the dimer. By designing a hetero dimer it was possible to change positions in the interface with the third monomer to disfavour binding while the equivalent position in the other monomer that is part of the dimer interface could remain unchanged. In addition to selected mutations to disfavor trimer formation, other mutations were introduced to make the newly exposed surface more hydrophilic.

Dimer 1 was predicted to destabilize trimer formation by ˜8 kcal/mol. Trimer destabilization by dimer 2 was predicted to be ˜5 kcal/mol. FoldX calculation also showed that dimer 2 was 2.8 kcal/mol more stable than dimer 1 and that the propensity to form homodimers was also energetically unfavourable (table 3).

TABLE 3
FoldX calculated ΔΔG stability energies
for the murine dimeric RANK variants.

| variant | kcal/mol | |
|---|---|---|
| D1 | 2.5 | heterodimer |
| D1-AA | 3.6 | homodimer |
| D1-BB | 7.4 | homodimer |
| D2 | −0.3 | heterodimer |
| D2-AA | 0.8 | homodimer |
| D2-BB | 5.8 | homodimer |
| D1-ABC | 8.2 | trimer |
| D2-ABC | 5.3 | trimer |

Table 3 legend: ΔΔG stability energies are calculated for the heterodimers D1 (mD1) and D2 (mD2) (consisting of a monomer A and B). In addition stability energies for putative D1 and D2 homodimers (consisting of two monomers A or two monomers B) are also depicted, showing that these homodimers are less energetically favorable than the heterodimers. The last two rows depict the destabilization of the murine RANKL trimer (monomers A, B and C) upon introducing the D1 or D2 mutations in monomer A and B, showing a clear destabilization of the trimer structure. Stability energies are in kcal/mol.

### Example 2

### Site Directed Mutagenesis, Expression and Purification of Dimers mD1 and mD2

Mutations were introduced using several round of site directed mutagenesis in a cDNA encoding soluble murine RANKL (aa 160-316). (18) The mutated PCR product corresponding to monomer A was cloned into the first multiple cloning site (MCS) of a pCDF-Duet-1 bicistronic co-expression vector (Merck) using BamH1 and HindIII. Monomer B was cloned into the second MCS using NdeI and XhoI. Monomer A is fused to a hexahistidine-tag to aid purification.

A recombinant negative E. coli BL21 derivate, E. coli BL21 BLR, was transformed with the expression vector encoding mD1 and mD2. The E. coli BL21 BLR cells were grown in 2×LB medium at 37° C. and 250 rpm until OD 0.5 was reached. Streptomycin (100 ug/mL) (Duchefa) or spectomycin (100 ug/mL) (Duchefa) was used as selective agent. After reaching OD 0.5 the cultures were induced with 1 mM Isopropyl β-D-1-thiogalactopyranoside (IPTG) (Duchefa) and the temperature was lowered to 20° C. The cells were incubated overnight and subsequently harvested by centrifugation at 10,000 g for 15 minutes. The pellet was washed with PBS and resuspended in 20 mM sodium phosphate (Merck) buffer, containing 10% glycerol (Duchefa), 150 mM NaCl (Merck), and 7 mM β-mercaptoethanol (Sigma), pH 7.5 (buffer A).

Cells were disrupted by sonicating the resuspended pellet for three minutes. The extract was clarified by centrifugation at 42,000 g for one hour.

The supernatant was loaded on a Ni Sepharose HP Column (HisTRAP HP GE Healthcare). The column was washed with buffer A and the his-tag containing dimer was eluted with a gradient 0-100% of elution buffer (buffer A containing 1 M imidazol (Sigma) pH 7.5). By having only monomer A tagged and using a His-tag purification step, presence of B can only be explained by complex formation between A and B. Fractions containing his-tag protein were combined, concentrated and loaded on a gelfiltration column (Superdex 75 16/60 GE Healthcare). Buffer A was used as elution buffer. Purified proteins were >90% pure as determined using a collodial Coomassie Brilliant Blue-stained SDS-PAGE gel. Purified protein solutions were flash frozen in liquid nitrogen and stored at −80° C.

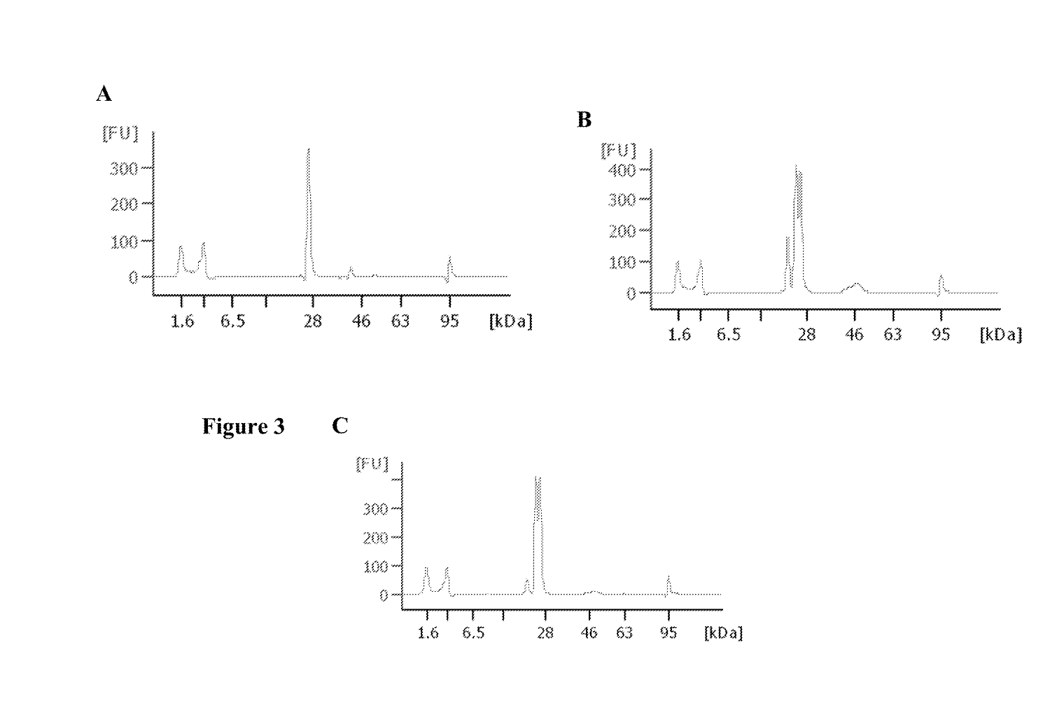

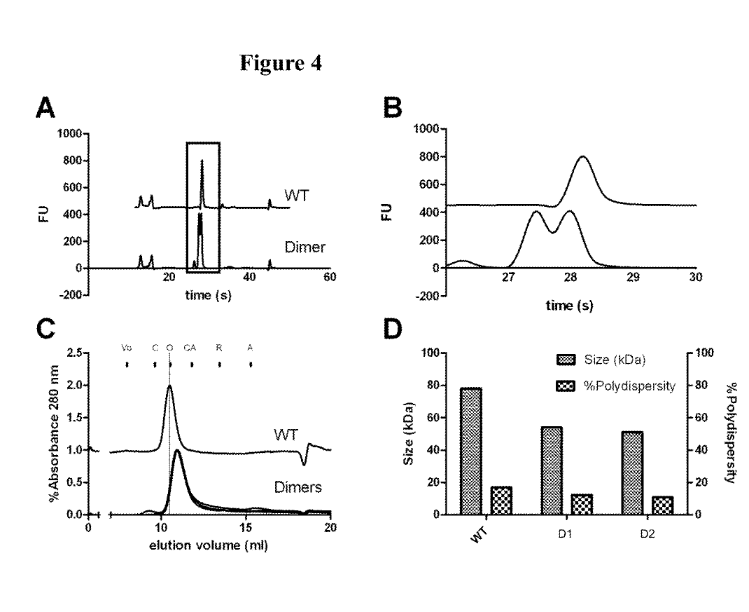

Mass spectroscopy (MALDI and MS-MS peptide sequencing) confirmed that both monomers of the heterodimer were present in the purified samples. For each variant, peptides with molecular weights corresponding to 19.2 kDa for His-tagged monomer A and 17.7 kDa for monomer B could be detected. Analysis by capillary electrophoresis also showed (FIG. 3 and FIGS. 4A and B) that monomer A and monomer B were present in the same quantities. Capilliary electrophoresis was carried out under denaturing conditions and showed that flag-mRANKL WT contains a single main peak, while mD1 and mD2 showed two equally sized, neighbouring peaks (FIG. 3 and FIGS. 4A and B). This is in agreement with the small—1.5 kDa—size difference between His-tagged monomer A and monomer B.

### Example 3

### Biophysical Characterization and Conformation of Stoichiometry

Circular Dichroism and thermal decay experiments were performed as described before (26). Samples were dialyzed against 20 mM Sodium phosphate buffer containing 250 mM NaCl and 10% glycerol pH 7.3 and were measured at 0.2 mg/mL.

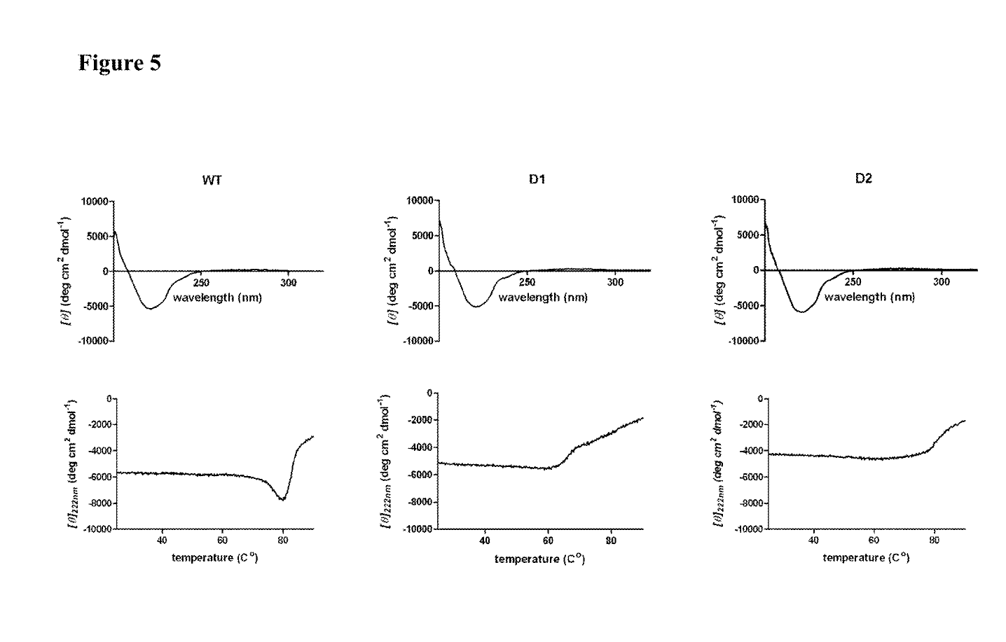

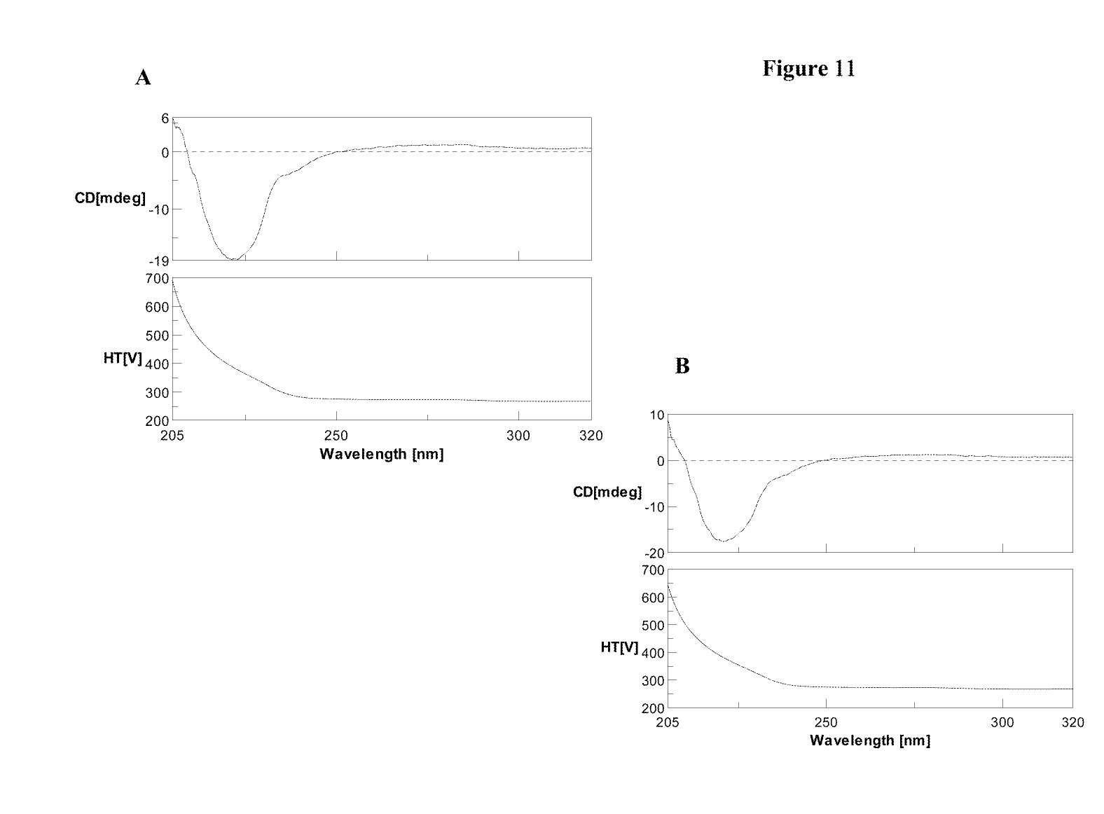

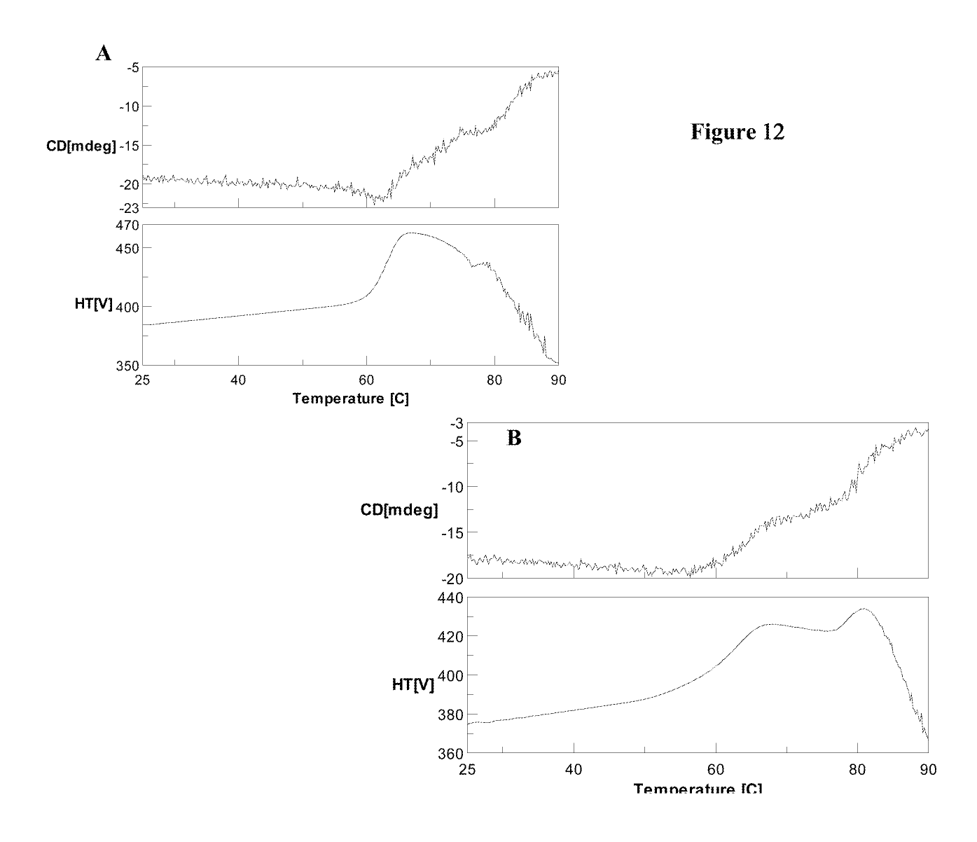

Circular dichroism measurements showed that the far UV (320-200 nm) CD spectrum of the dimers is similar to the CD spectrum of flag-mRANKL WT and indicated that both dimers are properly folded and have secondary structures consistent with an all β-sheet protein (FIG. 5 top panel and FIG. 11). Heat induced unfolding showed that both variants were structurally stable up to 60° C. (FIG. 5 lower panel and FIG. 12). mD2 as predicted by FoldX appears to be slightly (3-5° C.) more stable than mD1 (FIG. 5).

Analytical gelfiltration was performed with 100 μL sample with a concentration of 0.6 mg/mL. Samples were run 10/30 75 (GE Healthcare). The column was calibrated with protein standards of known size. Buffer A was used as elution buffer with a flow rate of 0.5 or 1 mL/min. The retention time was related to the retention times of the calibration standards, as shown in Table 4, below.

TABLE 4
Elution volumes from Gelfiltration assay

| | Vₑ (ml) | Kₐᵥ | ln(x) | MW (calc) |
|---|---|---|---|---|
| Dimers | | | | |
| dRANKL D2 + flag | 10.91 | 0.19 | 10.64 | 41821.92 |
| dRANKL D2 | 10.98 | 0.19 | 10.61 | 40596.64 |
| dRANKL D2 | 10.96 | 0.19 | 10.62 | 40943.01 |
| Trimers | | | | |
| flagRANKL WT | 10.5 | 0.16 | 10.82 | 49778.63 |
| flagRANKL WT | 10.5 | 0.16 | 10.82 | 49778.63 |
| flagRANKL WT | 10.49 | 0.16 | 10.82 | 49990.53 |

dRANKL D2 is mD2; flagRANKL WT is murine flagRANKL WT

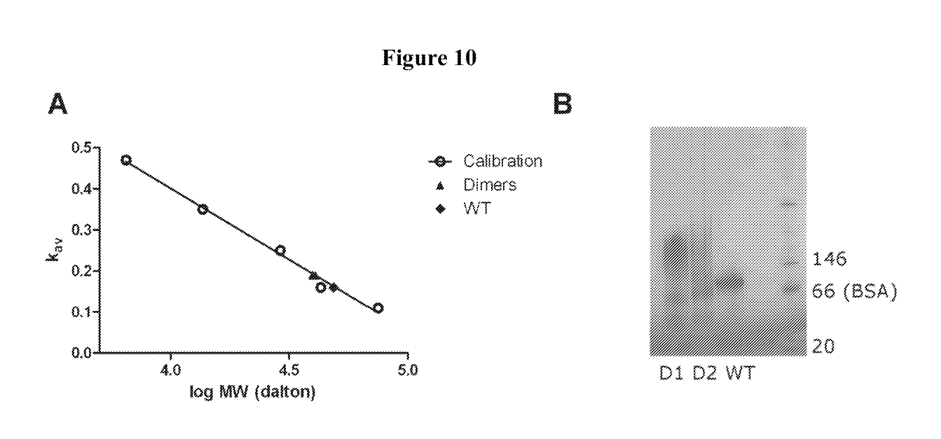

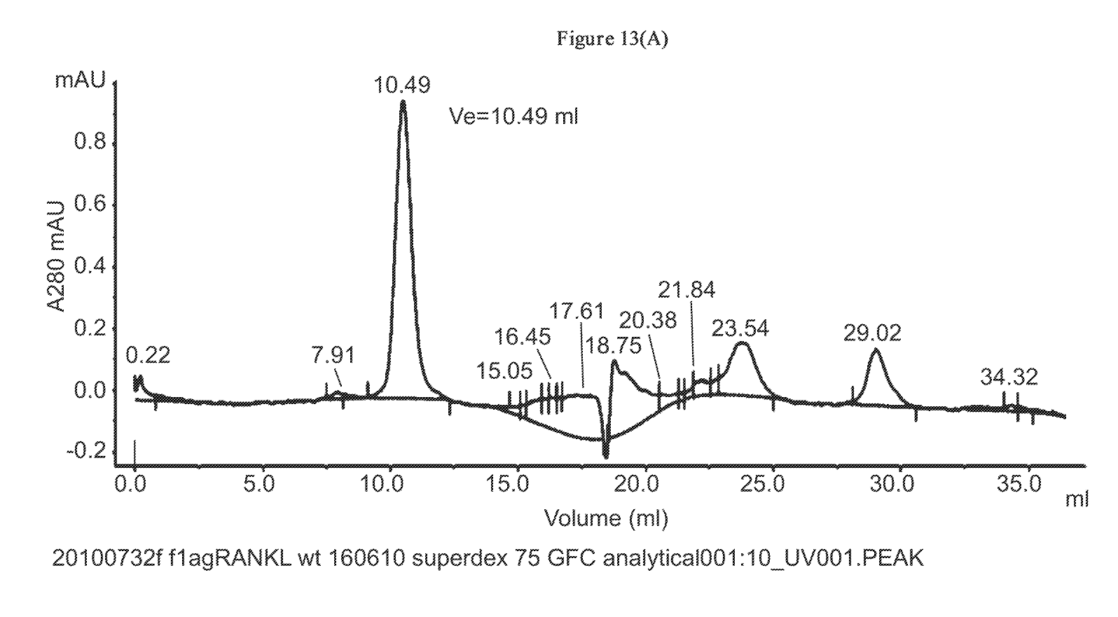

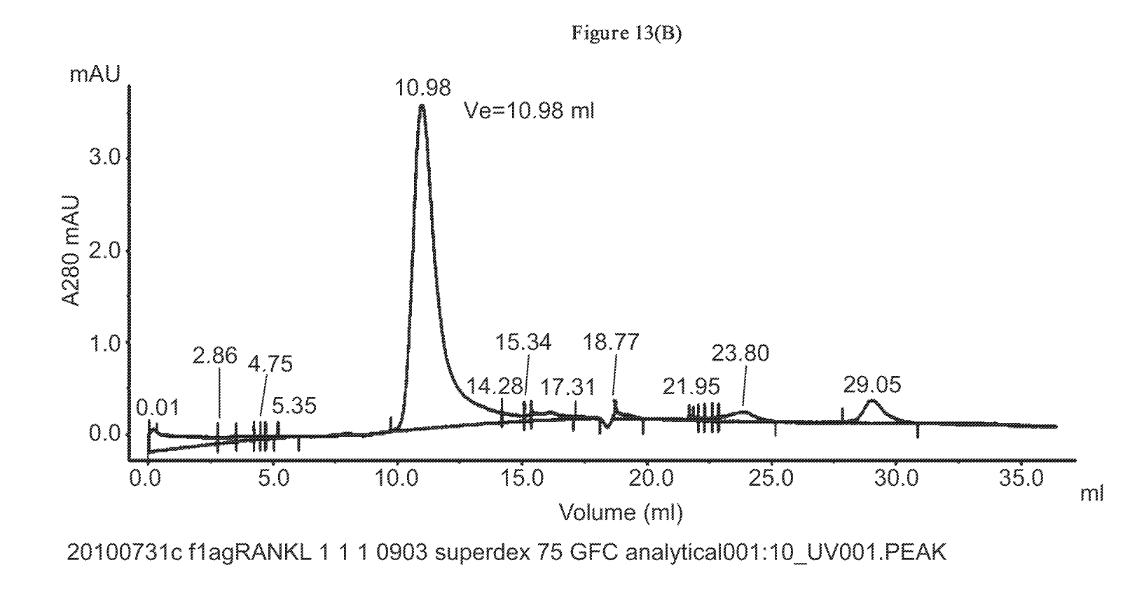

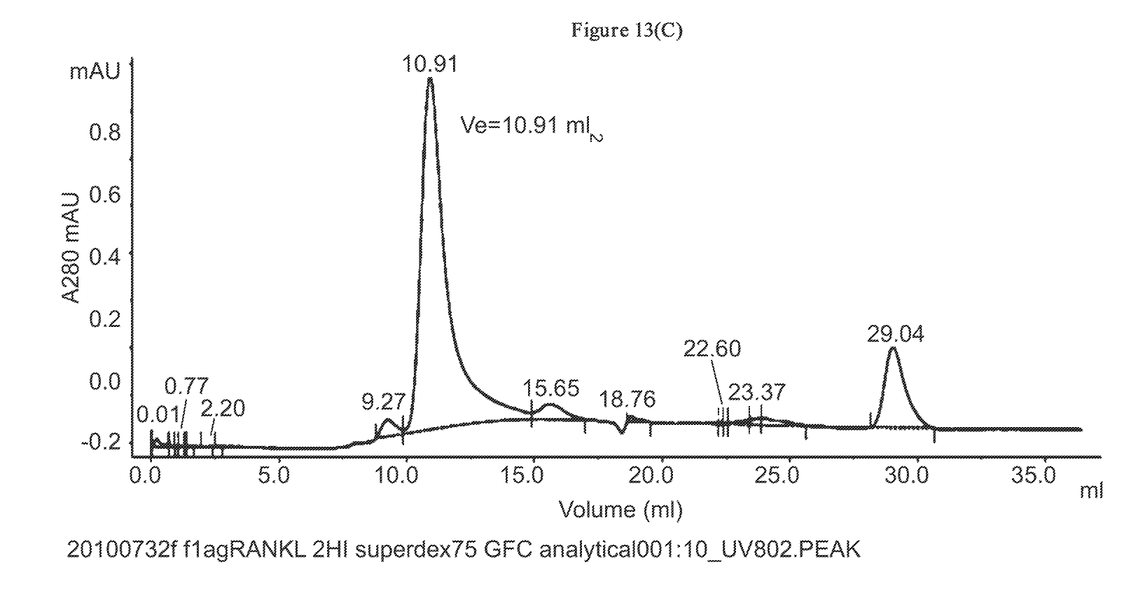

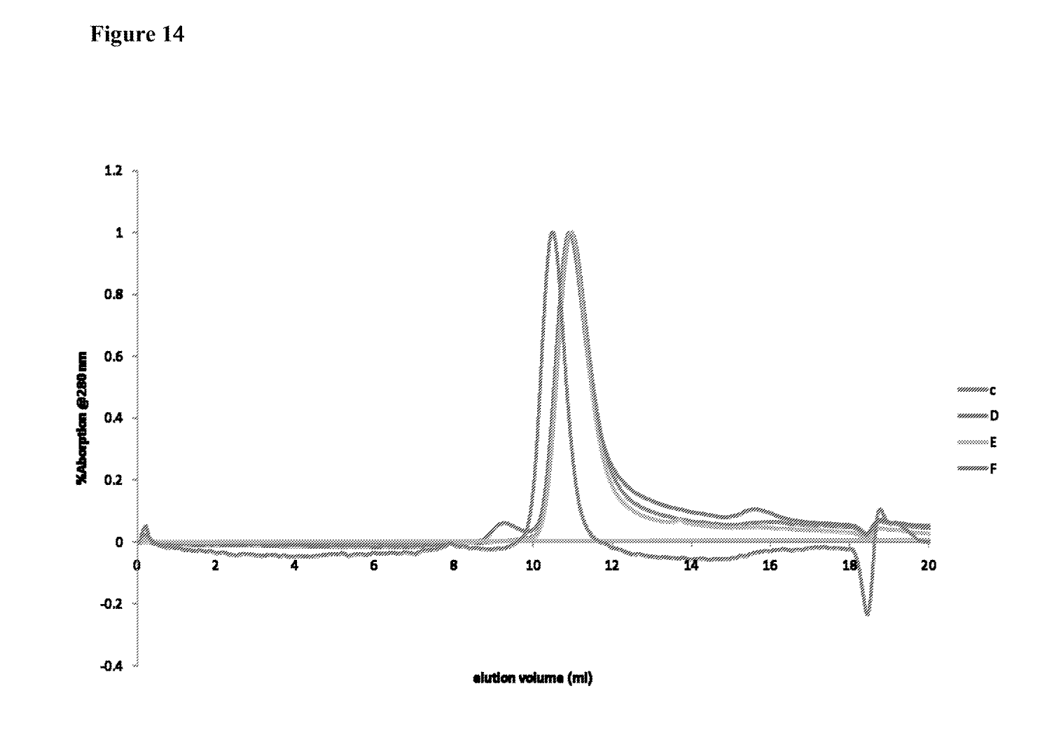

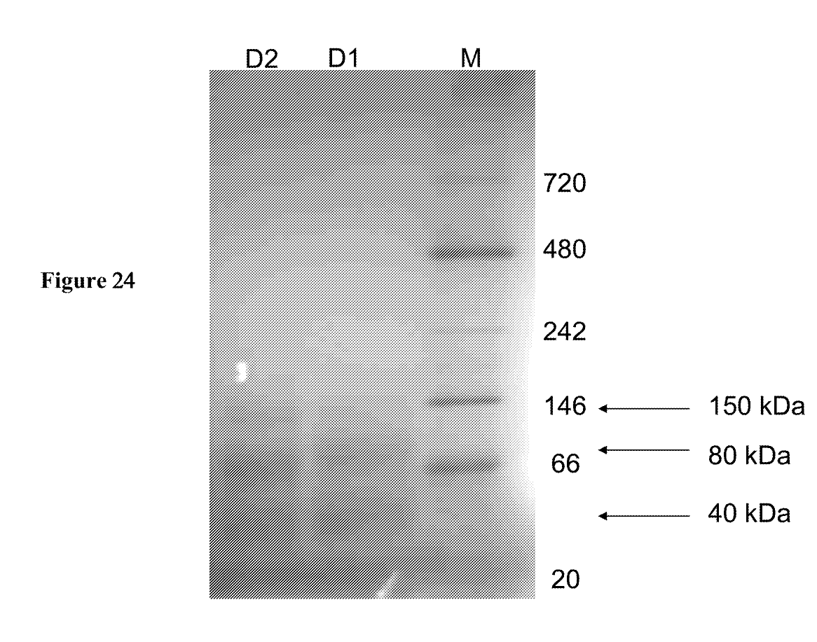

Analytical size exclusion chromatography showed that the dimer 1 (mD1) and dimer 2 (mD2) are not in the trimeric form as the elution volume is increased relative to flag-mRANKL wt (FIGS. 4C, 10A, 13 and 14). To confirm this a blue native PAGE was performed, the native gel showed a band at 35-40 kDa; the calculated size for the dimeric variants, but also two bands above 66 kDa suggesting some aggregation. No bands observed where wild-type mRANKL (54 kDa) would be expected or where monomers should run (18 kDa) (FIG. 10B and FIG. 24).

Samples were also analyzed by Dynamic Light Scattering (DLS). Purified samples (0.5-2 mg/mL) were before measurements filtered over a 0.2 μm filter. 20 measurements per sample were recorded. Data analysis was performed using Dynapro software, the data was analyzed as being bimodal. Blue native gel electrophoresis (NativePage 12% Invitrogen) was performed according to manufacturers instructions.

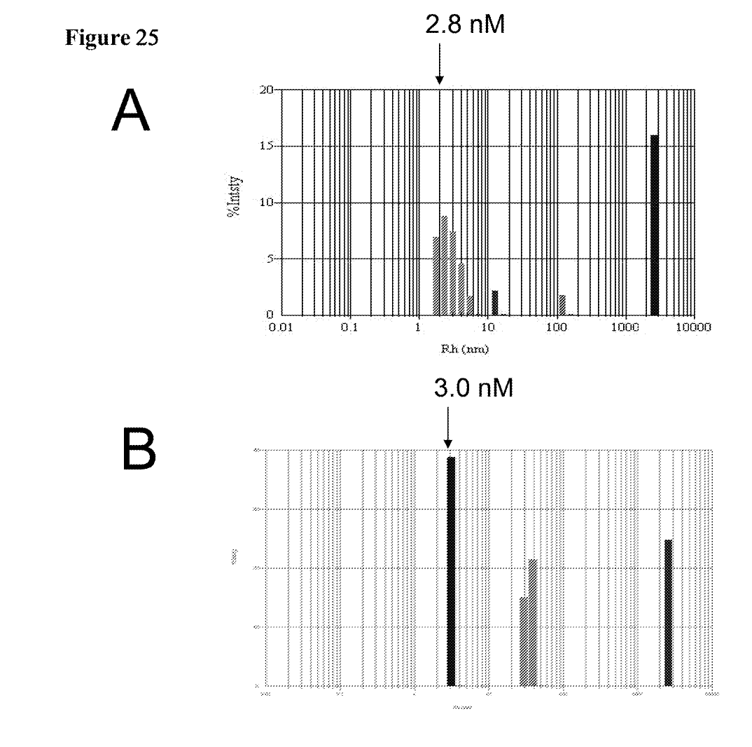

The calculated values from the DLS experiment are 38 kDa and 46 kDa for dimer 1 (mD1) and dimer 2 (mD2), respectively (Figure FIG. 4D and FIG. 25). While RANKL dimer 2 shows a minimal polydispersity (<0.1%), the polydispersity of dimer 1 was considerably larger at 35%. This is in line with the possible aggregation as observed in the BN-PAGE experiment described above. As for size exclusion chromatography, the mass calculated in the DLS experiments depends on the shape of the protein and this might explain why the values are slightly higher than would be expected (29).

### Example 4

### Binding of RANKL Variants D1 and D2 to Only One Receptor Molecule

A Surface Plasmon Resonance receptor binding assay was used as a shape independent method to determine the molecular weight of the RANKL dimers. In contrast to GFC and DLS which measure the oligomerization state of relatively concentrated samples (>100 ug/ml), this assay has the added benefit that it measures the MW; and hence the oligomerization state, at physiologically relevant concentrations (<1 ug/ml). The maximum response as recorded in a SPR assay depends on the MW and the amount (in response units [RU]) of the receptor coupled to the sensor chip and the MW of the analyte when observing a 1:1 binding (Equation 1).

$${RU}_{Ligand ( Maximum )} = \frac{{MW}_{Ligand}}{{MW}_{Receptor}} \times {RU}_{ReceptorCaptured} .$$

Binding parameters were determined using a Surface Plasmon Resonance based assay on a Biacore200 system (GE Healthcare). A C1 chip (GE Healthcare) was coated with 1000 response units of protein A (Sigma) using standard amine coupling chemistry as described before (15,27,28).

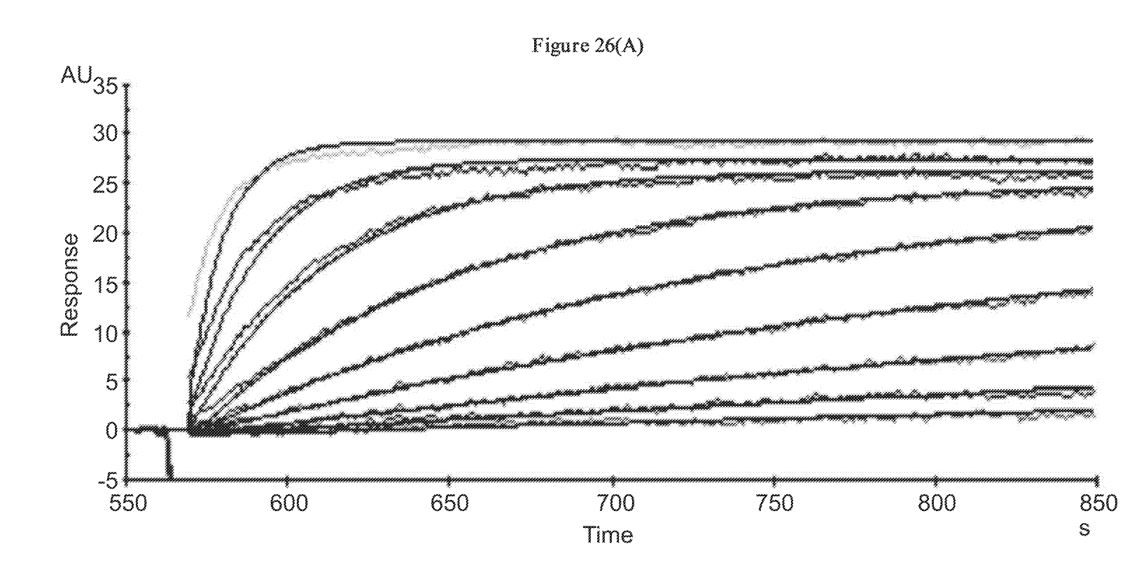

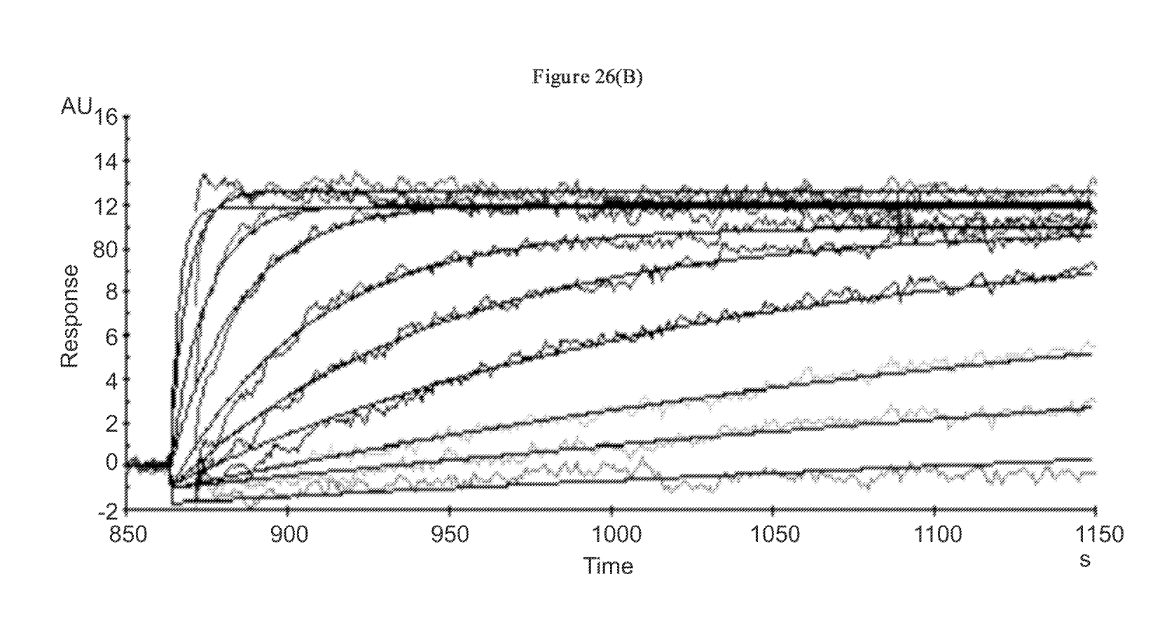

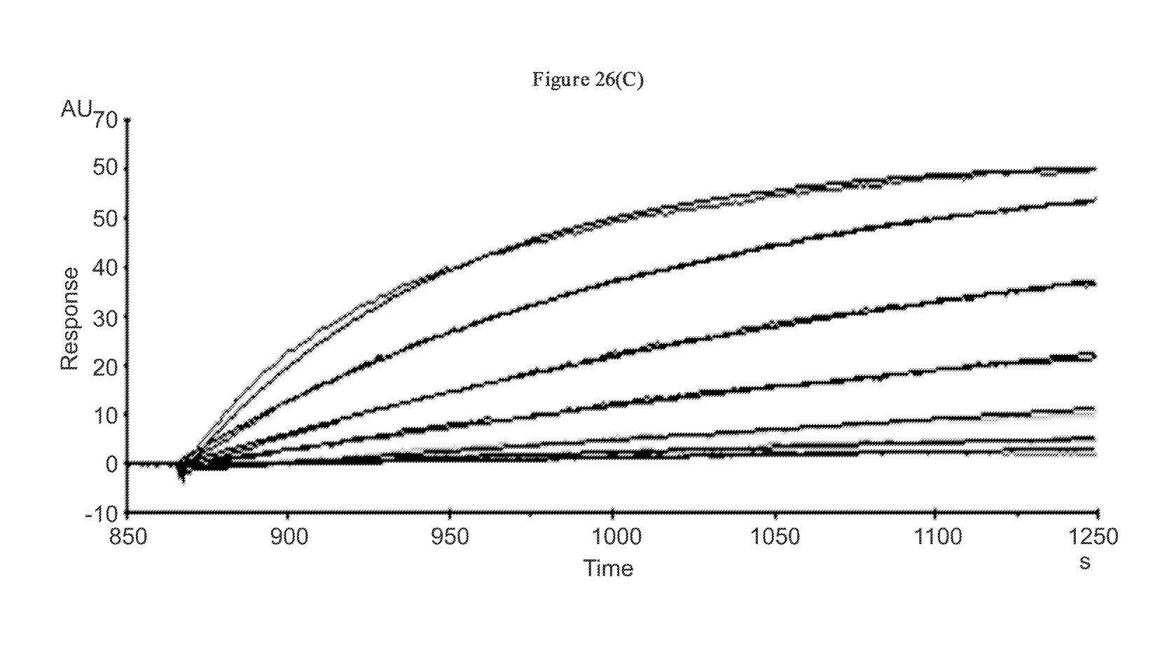

The calculated Rₘₐₓ for dimeric RANKL variant mD1 at a receptor capture level of hRANK-Fc of 57 RU is 34 RU and for dimeric variant mD2 at a capture level of 25 RI is 15 RU. The measured levels are 30 RU and 14 RU, respectively. The measured Rₘₐₓ for mRANKL wild-type is (65 RU) at a capture level of 37 RU. Thus the measured values of the dimers and wt closely agree with the calculated theoretical values. Hence, the oligomerization state of the RANKL variants is dimeric (FIG. 26).

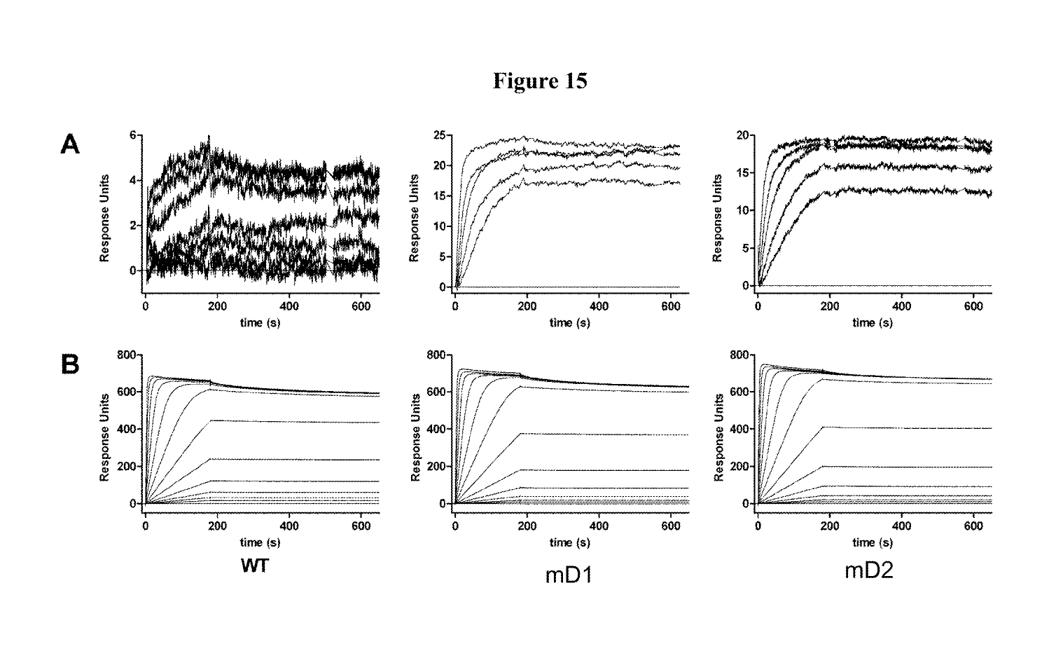

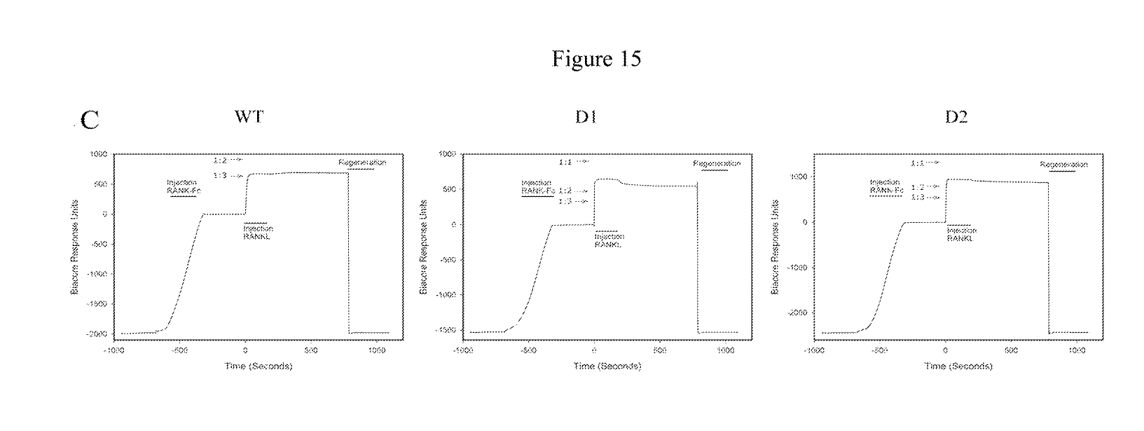

The same experiment was performed with murine RANKL WT and mRANK-FC. At very low receptor density, RANKL proteins bind only one receptor molecule, allowing the data to be fitted to a Langmuir 1:1 interaction model (52). mRANKL WT needed a very low density (<20 resonance units (RU)) to come close to a 1:1 ratio (FIG. 15A). FIG. 15A shows typical sensorgrams of flag-mRANKL WT binding to only 7.5 RU of captured mRANK-Fc. Maximum binding of 5.5 RU is reached, which is slightly lower than the expected Rₘₐₓ value of 6.8 RU for a 1:1 ratio, but definitely larger than 3.4 RU, the Rₘₐₓ value expected for a binding ratio of 2 molecules of RANK per flag-mRANKL WT trimer. Similar to RANKL WT, binding of RANKL mD1 or mD2 to mRANK-Fc results in slightly lower Rₘₐₓ values than expected for a 1:1 binding ratio: 24 RU D1 compared to 26 RU, and 20 RU D2 compared to 23 RU (FIG. 15A). These binding ratios could be reached with mD1 and mD2 at densities of mRANK-Fc of <50 RU. The approximate 1:1 binding of RANKL proteins to mRANK-Fc allows fitting the data to the 1:1 Langmuir binding model. The trimeric nature of members of the TNF ligand family makes it difficult to measure binding kinetics at high receptor densities. However, such measurements can provide information on the ratio of molecules in the formed complex. SPR measurements were performed using ˜1500-2000 RU of captured mRANK-Fc on the surface of a CM5 sensor chip (FIGS. 15B and C). This is a density that results in a mean ratio of RANKL:RANK of approx 1:3 (52). Indeed, the results of flagRANKL WT show that this ratio is nearly reached since the apparent Rₘₐₓ value, obtained after injecting 500 nM of flag-mRANKL WT corresponds to one trimeric molecule of flag-mRANKL WT binding to 2.8 monomeric subunits of RANK-Fc. (Table 5, below) In contrast, the ratio of binding of RANKL variants mD1 and mD2 to mRANK-Fc seems hardly to be affected by receptor density (Table 5). The values obtained after injecting 500 nM of the dimeric variants correlate to binding of 1.5 monomeric subunits by mD1 and 1.6 monomeric subunits by mD2. These findings suggest that the dimeric RANKL constructs lost the ability to bind three RANK-Fc receptor molecules, as was expected from the designs.

TABLE 5
RANKL-RANK stoichiometric ratio determination

| | Low Density | | | High Density | | |
|---|---|---|---|---|---|---|
| | RANK-Fc | Measured Rmax | Calculated | RANK-Fc | Measured Rmax | Calculated |
| | Capture level (RU) | RANKL (RU) | Stoichiometry | Capture level (RU) | RANKL (RU) | Stoichiometry |
| mWT | 7.5 | 5.5 | 1:1.28 | 1960 | 670 | 1:2.75 |
| mD1 | 45 | 24 | 1:1.15 | 1510 | 640 | 1:1.45 |
| mD2 | 38 | 20 | 1:1.17 | 2430 | 946 | 1:1.58 |

Table 5: Determination of the stoichiometric ratio of the interaction between murine RANKL WT (WT), mD1 or mD2 with immobilized murine RANK-Fc. At low receptor density levels RANKL WT, mD1 and mD2 form a 1:1 complex with RANK-Fc while at high receptor density the RANKL WT/RANK-Fc interaction approach a stoichiometric ratio of 1:3. In contrast, the stoichiometric ratio's for the interactions between mD1 or mD2 and RANK remain around 1:1.5. The following molecular weights were used for the calculation: 56.3, 36.9, 37 and 60 kDa for murine WT, mD1, mD2 and mRANK-Fc respectively.

### Example 5

### Determination of RANK-Fc and OPG Binding

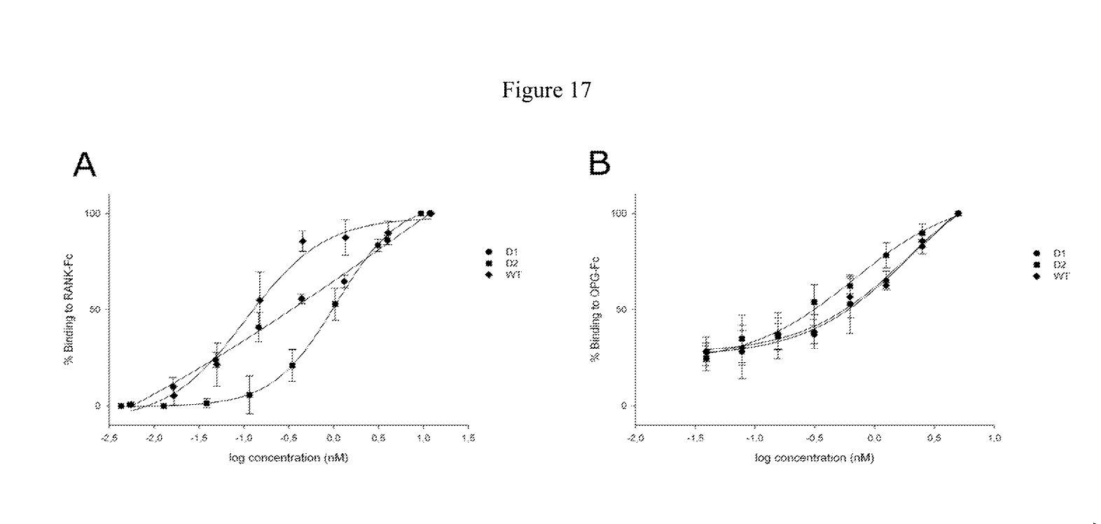

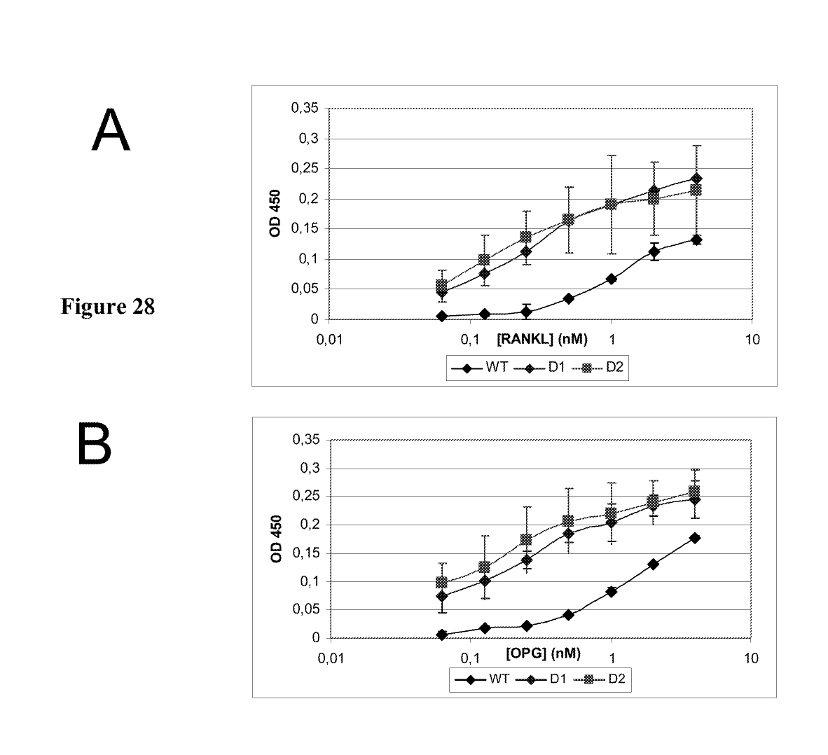

To demonstrate that RANKL dimers were still able to bind human RANK or human OPG with high affinity an ELISA experiment was performed. Wells were coated with hRANK-FC or hOPG-FC fragments and a concentration range of the RANKL dimers was added. Both dimer 1 (mD1) and dimer 2 (mD2) still bound to hRANK-FC with similar high affinity, as shown in (FIG. 28A) Binding was shown to be concentration dependent, Similar results were observed for hOPG binding; both mD1 and mD2 bind in a concentration dependent fashion with similar affinities to OPG (FIG. 28B). In addition to hRANK-Fc and hOPG-Fc, the dimers mD1 and mD2 also were still able to bind to (A) murine RANK-Fc and (B) murine OPG-Fc (FIGS. 17A and B).

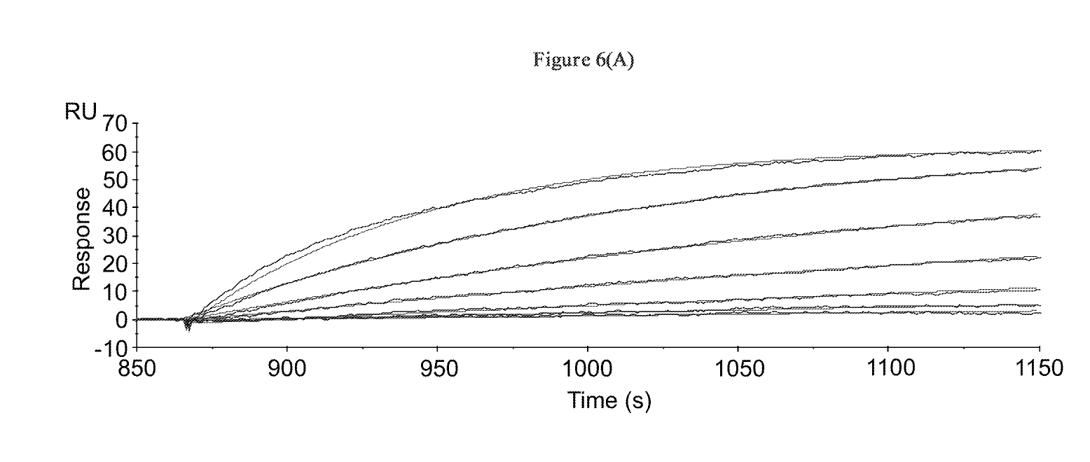

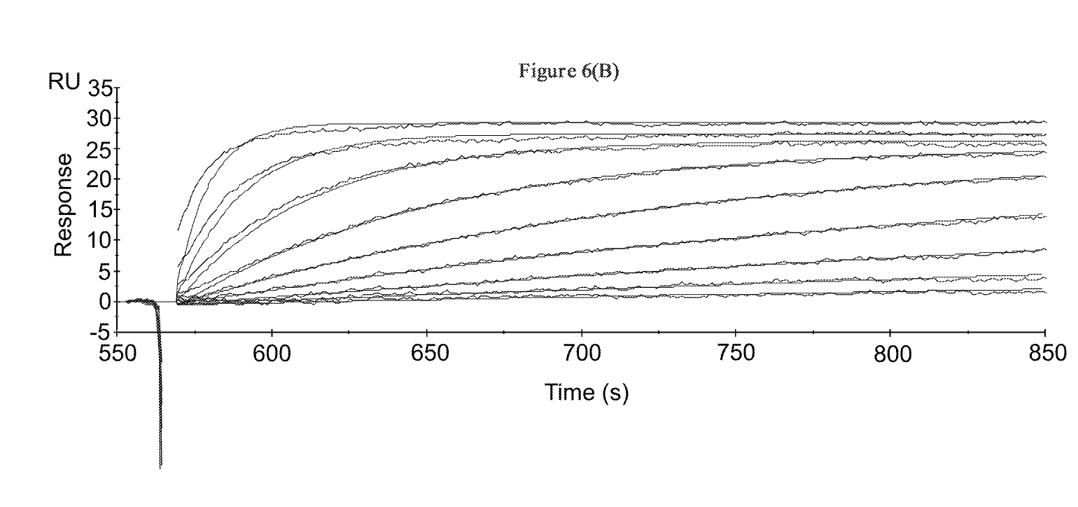

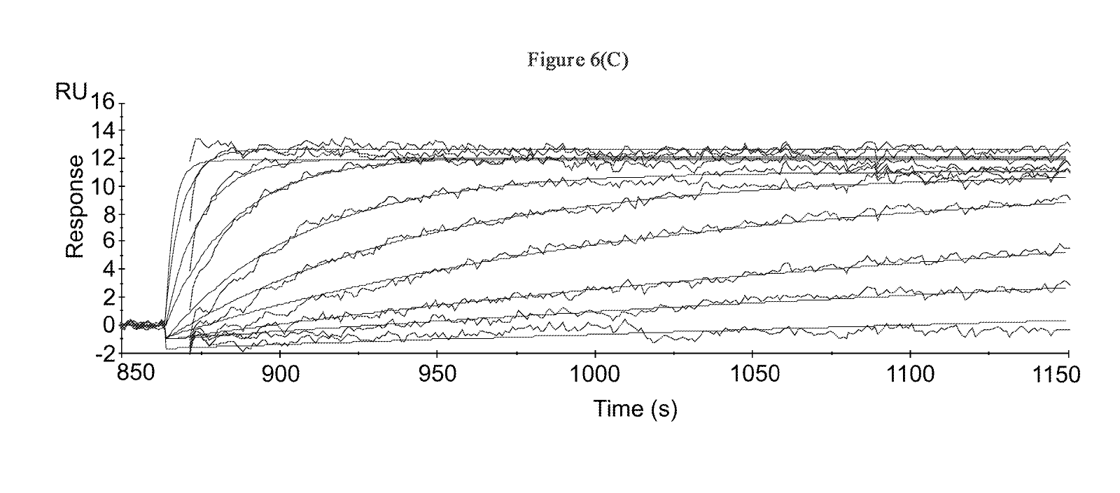

A Biacore experiment was performed to determine the kinetics of binding of the dimeric variants. hRANK-FC (Sigma) was captured in flow cell 2 at a level of 40 RU by a 60 s injection pulse of a 0.7 ug/ml hRANK-FC solution at a flow rate of 50 uL/min. Flow cell 1 served as reference cell. Subsequently, serial dilutions ranging from 562 nM to 100 pM of murine RANKL wt (R&D systems), or the purified RANKL dimers mD1 and mD2 were injected over both flow cells at 50 uL/min and 37° C. Binding was measured in real time. After each cycle the protein A surface was regenerated with a 30 s pulse of 10 mM Glycine pH 1.5. K_{d} values were calculated by fitting the data to a langmuir 1:1 binding model using the BIAevaluation software as described before (30). (Table 6 and FIG. 6).

TABLE 6
Binding kinetics of mD1, mD2 and trimeric
murine wtRANKL to hRANK-Fc as shown by SPR

| | kₐ (1/Ms) | k_{d} (1/s) | K_{A} (1/M) | K_{D} (pM) |
|---|---|---|---|---|
| mD1 | 4.52 (+/−1) E6 | 1.97 (+/−0.5) E−4 | 2.33 (+/−0.7) E10 | 44.6 (+/−7) |
| mD2 | 8.55 (+/−1.5) E6 | 5.54 (+/−2.5) E−4 | 1.80 (+/−0.8) E10 | 61.9 (+/−25) |
| mWT | 8.27 (+/−1.8) E5 | 8.97 (+/−1.74) E−5 | 9.23 (+/−0.2) E9 | 110 (+/−4) |

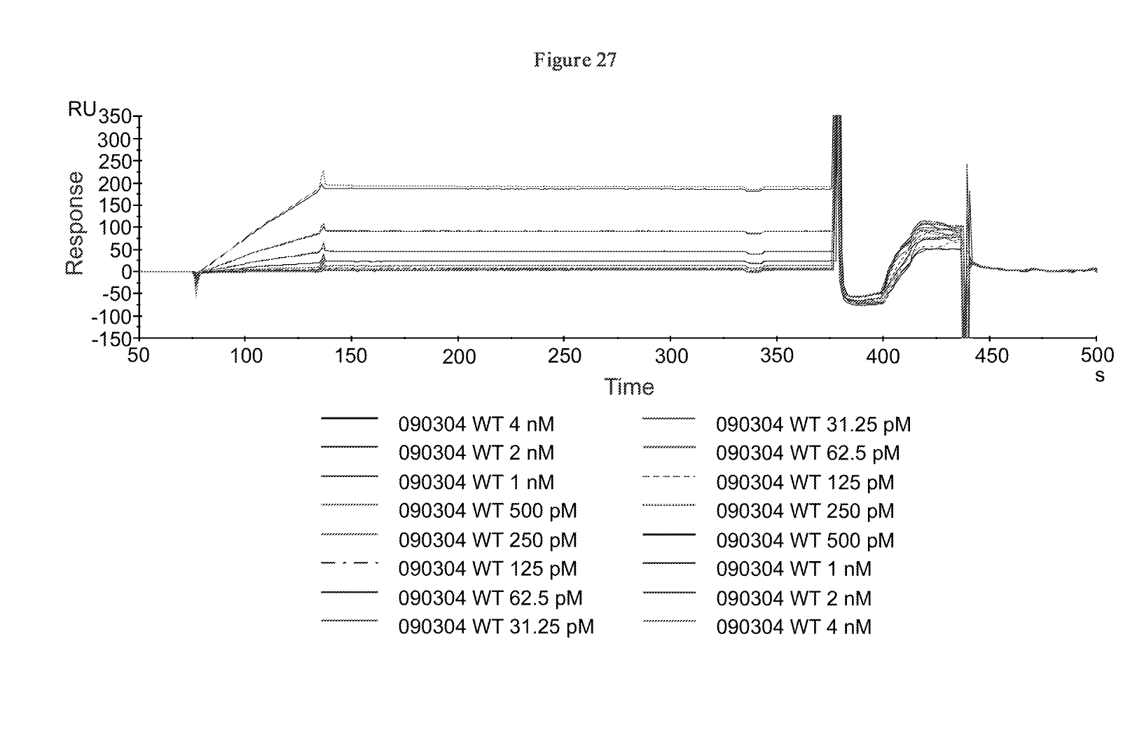

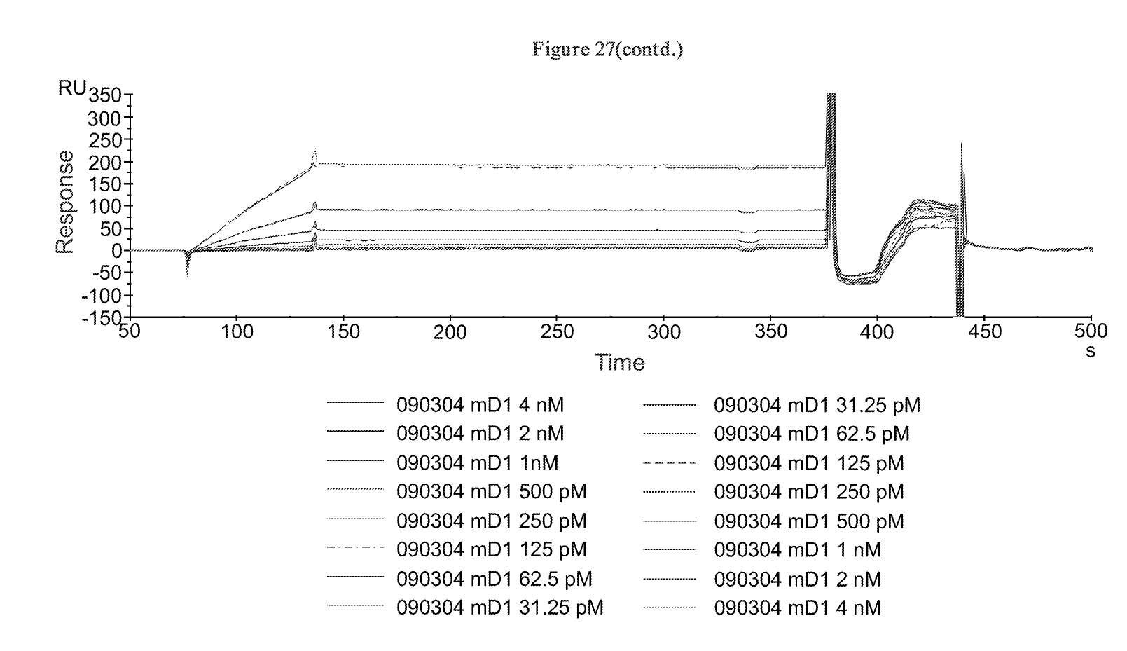

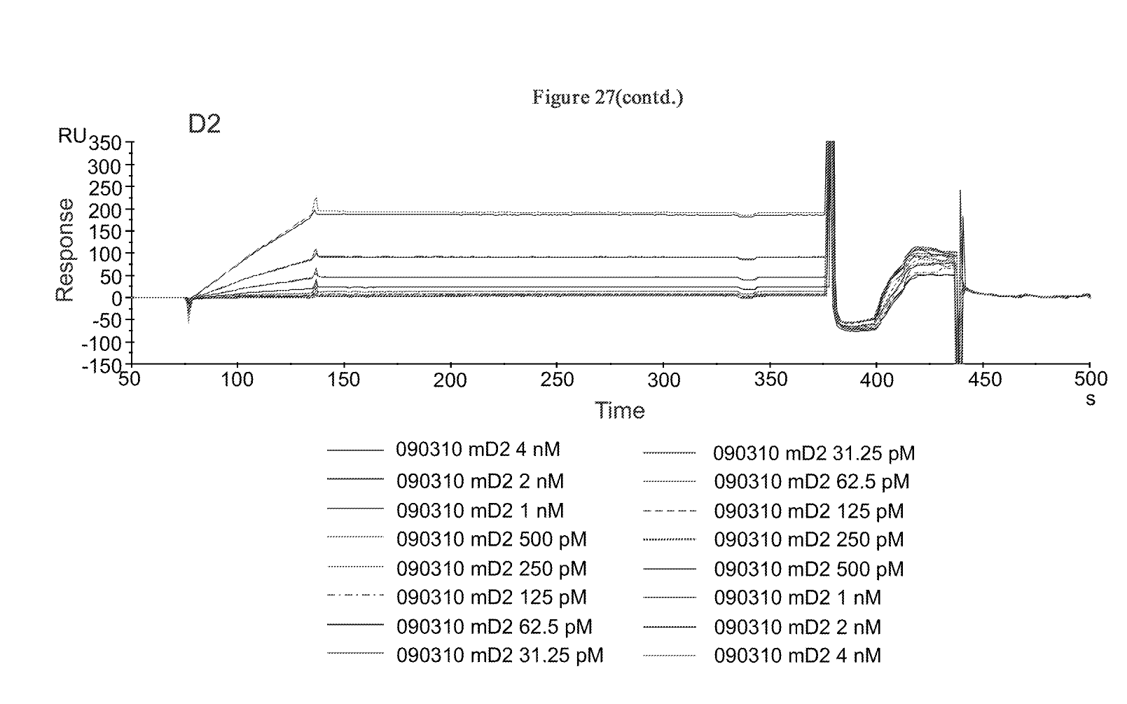

hOPG-Fc (R&D systems) was immobilized directly on the surface of a CM5 chip (GE Healthcare) using a standard amine coupling procedure according to manufacturer's instructions. RANKL dimers mD1 and mD2 or murine RANKL WT were injected at 4 nM at a flow of 70 uL/min and binding was measured in real time. The temperature was set at 25° C. during the measurements. Between cycles the surface was regenerated with a 30 s injection of 10 mM Glycine, pH 1.5. Association and dissociation was visualized with BIAevaluation software from Biacore (FIG. 27).

Kinetic binding parameters were measured in real time using SPR. RANKL dimer binding curves were recorded using concentrations ranging from 100 pM to 562 nM dimer 1 (mD1) or dimer 2 (mD2), trimeric murine wild-type RANKL was used as a control. Measurements were performed at 37° C. The dissociation constants (K_{D}) were determined by fitting the data to a Langmuir 1:1 interaction model. The K_{D} values for wild-type, dimer 1 and dimer 2 were 110 (+/−40) pM, 45 (+/−7) pM and 62 (+/−25) pM, respectively (table 6). The association rate constants (kₒₙ) were ˜5 fold (dimer 1) and ˜10 fold (dimer 2) faster than the association rate constant of wild-type murine RANKL, as shown in Table 6, above. The binding off-rate constant (k_{off}) of wild-type murine RANKL was ˜2 to 6 fold slower than for dimer 1 and dimer 2. SPR binding measurements to OPG at 25° C. showed similar results as RANK binding (FIG. 27B).

Kinetic binding parameters were also measured for mD1 and mD2 binding to mRANK-FC in real time using SPR using murine RANKL-WT as a control (FIG. 15; table 8). RANKL dimer binding curves to mRANK-Fc captured at low density on the surface of a C1 sensorchip were recorded using concentrations ranging from 100 pM to 562 nM. At very low receptor density, RANKL proteins bind only one receptor molecule, allowing the data to be fitted to a Langmuir 1:1 interaction model (29). RANKL WT needed a very low density (<20 resonance units (RU)) to come close to a 1:1 ratio (FIG. 3A). FIG. 15A shows typical sensorgrams of flag-mRANKL WT binding to only 7.5 RU of captured RANK-Fc. Maximum binding of 5.5 RU is reached, which is slightly lower than the expected Rₘₐₓ value of 6.8 RU for a 1:1 ratio, but definitely larger than 3.4 RU, the Rₘₐₓ value expected for a binding ratio of 2 molecules of RANK per flag-mRANKL WT trimer. Similar to mRANKL WT, binding of RANKL mD1 or mD2 to mRANK-Fc results in slightly lower Rₘₐₓ values than expected for a 1:1 binding ratio: 24 RU D1 compared to 26 RU, and 20 RU D2 compared to 23 RU (FIG. 15A). These binding ratio's could be reached with mD1 and mD2 at densities of mRANK-Fc of <50 RU. The approximate 1:1 binding of RANKL proteins to mRANK-Fc allows fitting the data to the 1:1 Langmuir binding model. The association rate constants (kₐ) are comparable for flag-mRANKL WT, mD1 and mD2 (Table 2). The dissociation rate constants (k_{d}) differ more, but also have large standard deviations due to the slow and thus hardly measurable dissociation. The calculated K_{D} values of the WT and D1 and D2 variants are similar and in the subnanomolar range, all showing a high affinity towards the RANK receptor.

TABLE 7
Binding kinetics of mD1, mD2 and trimeric murine RANKL WT to mRANK-Fc

| | kₐ (1/Ms)^{a} | k_{d} (1/s)^{a} | K_{A} (1/M)^{b} | K_{D} (pM)^{b} |
|---|---|---|---|---|
| mWT | (5.9 ± 1.0) × 10⁶ | (3.4 ± 4) × 10⁻⁴ | (4.4 ± 3.8) × 10¹⁰ | (65 ± 80) |
| mD1 | (5.8 ± 5) × 10⁶ | (1.5 ± 1.2) × 10⁻⁴ | (2.7 ± 2.1) × 10¹⁰ | (146 ± 220) |
| mD2 | (4.9 ± 0.8) × 10⁶ | (4.3 ± 2.5) × 10⁻⁵ | (2.1 ± 1.8) × 10¹¹ | (10 ± 9) |

Table 7 - Binding kinetics of mD1, mD2 and trimeric murine RANKL WT to mRANK-Fc as shown by SPR.
^{a}mean values derived from three different experiments;
^{b}mean values of the three kₐ/k_{d} or k_{d}/kₐ quotients of these different experiments.

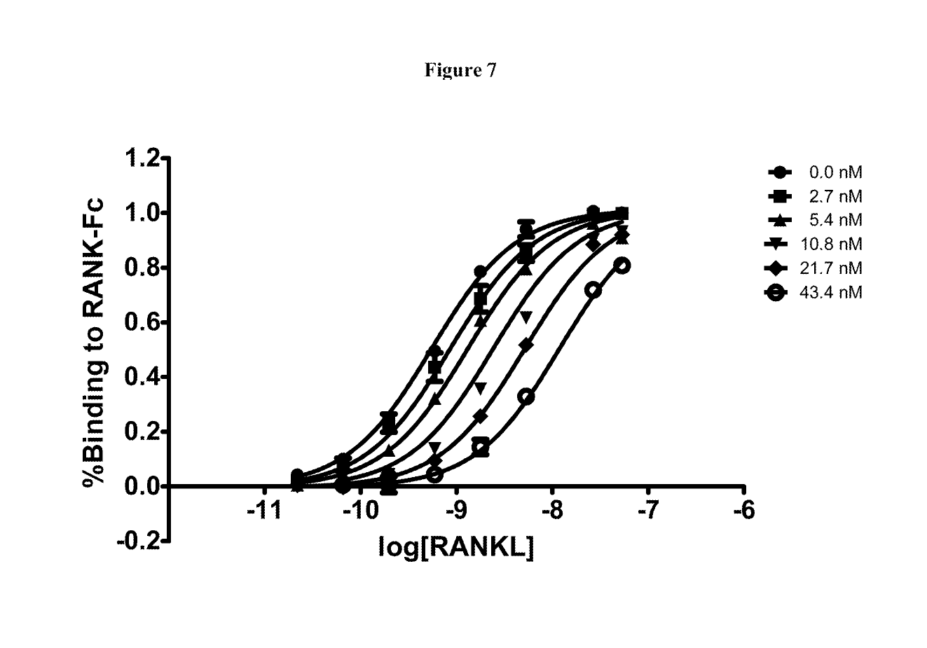

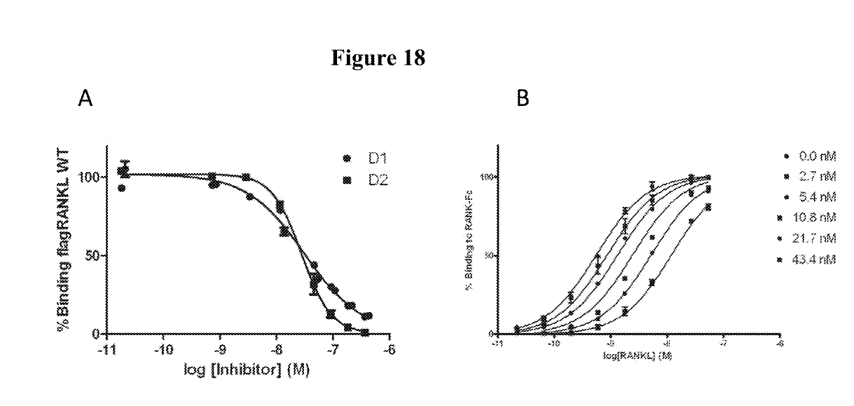

The ability of dimer 1 and dimer 2 to compete with wild-type murine RANKL for binding to mRANK was investigated using a competitive ELISA. Serial dilutions of wild-type mRANKL containing an N-terminal flagtag (flagRANKL) (0-53 nM) and a fixed concentration of dimer 1 (mD1) or dimer 2 (mD2) (0, 2.7, 5.4, 10.8, 21.7 or 43.4 nM) were added to RANK-Fc coated wells. After washing off unbound RANKL, mRANK bound flag-mRANKL was detected by an anti-flag antibody. A RANKL dimer concentration dependant decrease in mRANK binding and right shift in the EC₅₀ of flag-mRANKL was observed (FIGS. 7 and 18B), indicating a competitive inhibition based mechanism. IC₅₀ values for dimer 1 and dimer 2 were calculated to be 4 nM by a global fit of the individual dose response curves using a four parameter logistic model. No significant differences were revealed between dimer 1 (mD1) and dimer 2 (mD2).

### Example 6

### Inhibition of Osteoclastogenesis

To test the antagonistic properties of the RANKL dimers, the ability of RANKL dimers to block wild-type RANKL induced multinucleation of osteoclast precursor RAW 264.7 cells was determined.

Murine RAW 264.7 cells were cultured as described (19). The cells were sub-cultured every third or fourth day by a 1:10 dilution in fresh medium. Cells were seeded at a density of 1,000 cells/well in 100 μL in a 96 well plate. The wells bordering the edge of the plate were filled with 100 μL water to prevent evaporation of medium of wells containing cells. At day three the medium was changed for Alpha-Mem medium (Invitrogen) and RANKL dimers mD1 or mD2 or wt-mRANKL trimer (Antigenix America) were added at 50, 100 and 150 ng/mL. Inhibition of wtRANKL by dimer 1 or dimer 2 was measured by adding 100 ng/mL wtRANKL and 100 ng/mL of the dimeric variant. At day 5 the medium was replaced and wild-type or variant RANKL was added at the same concentrations.

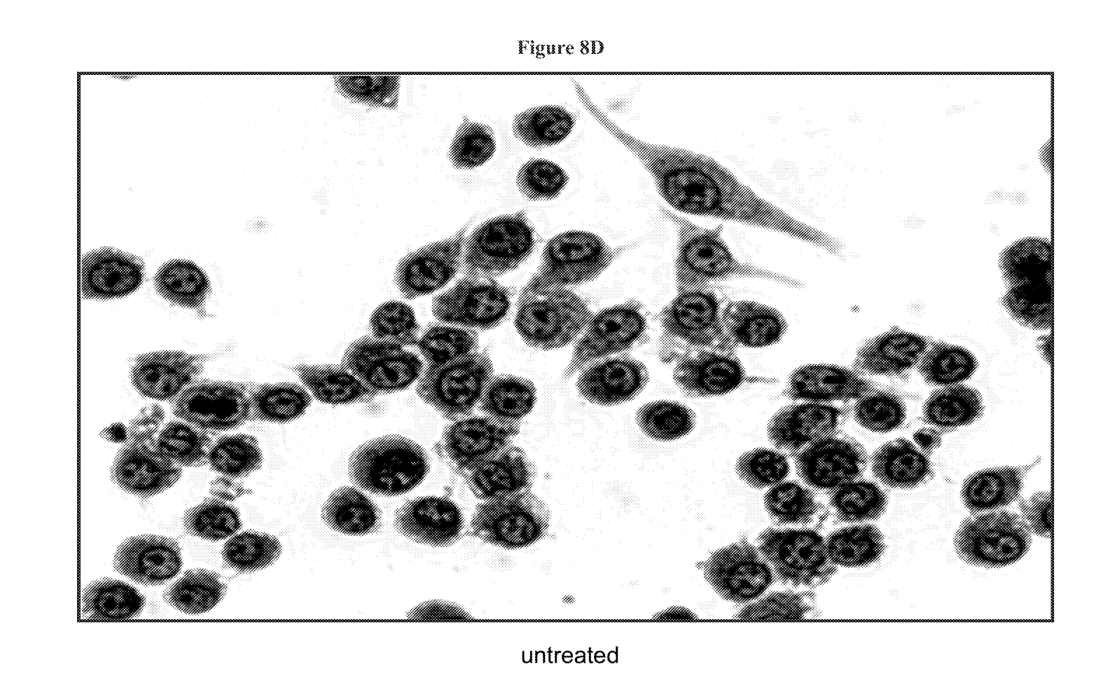

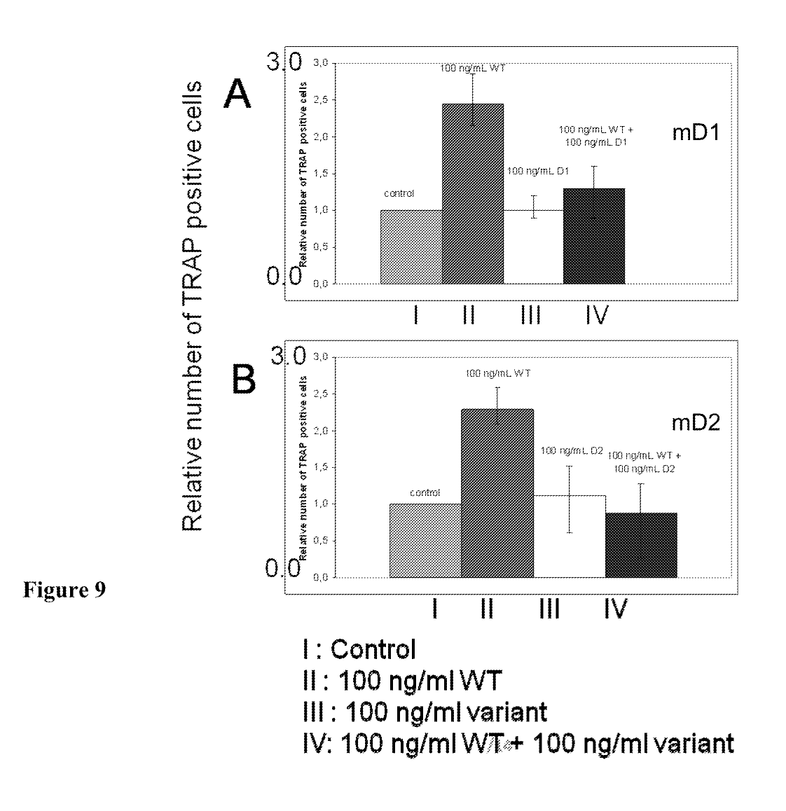

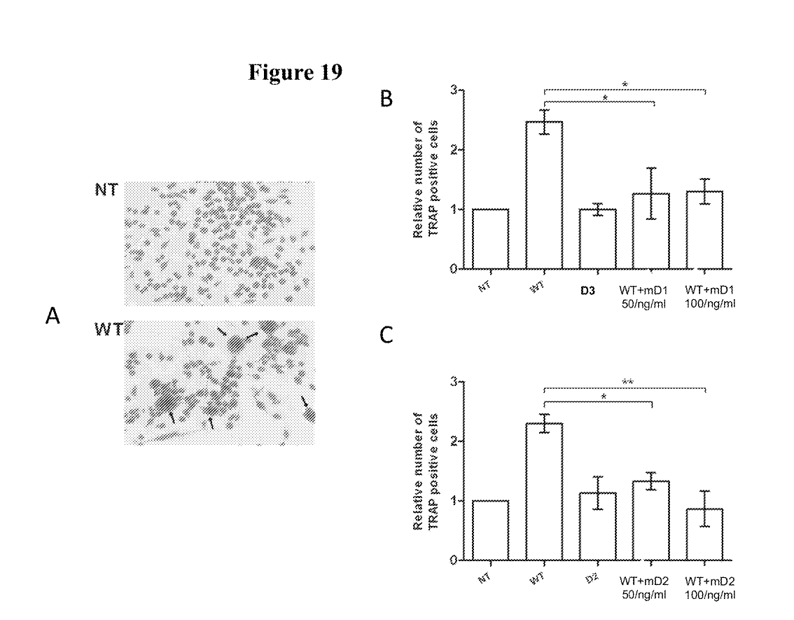

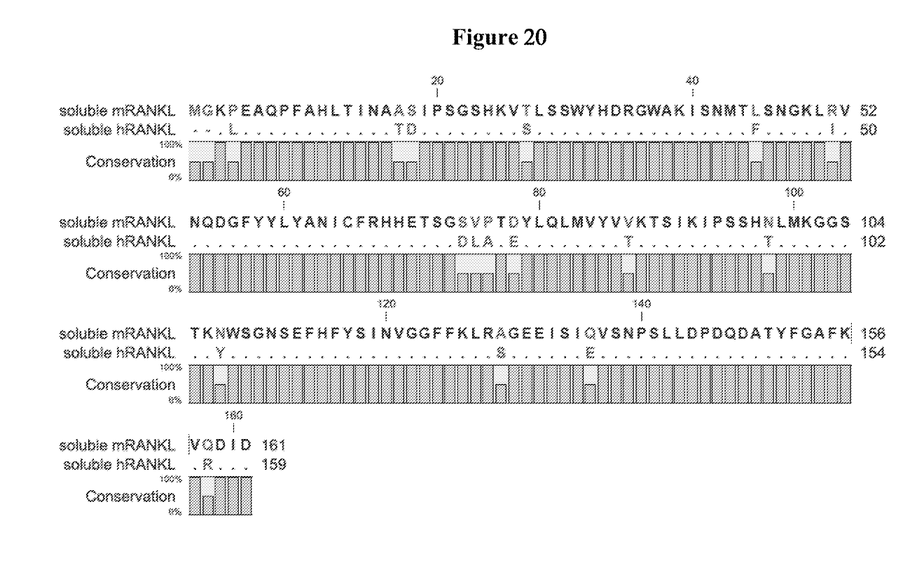

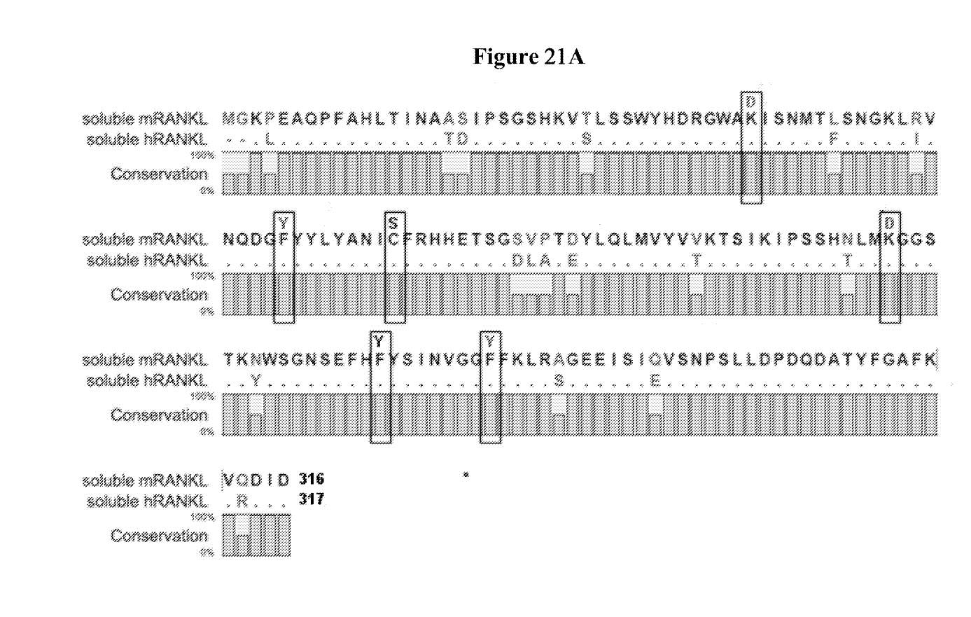

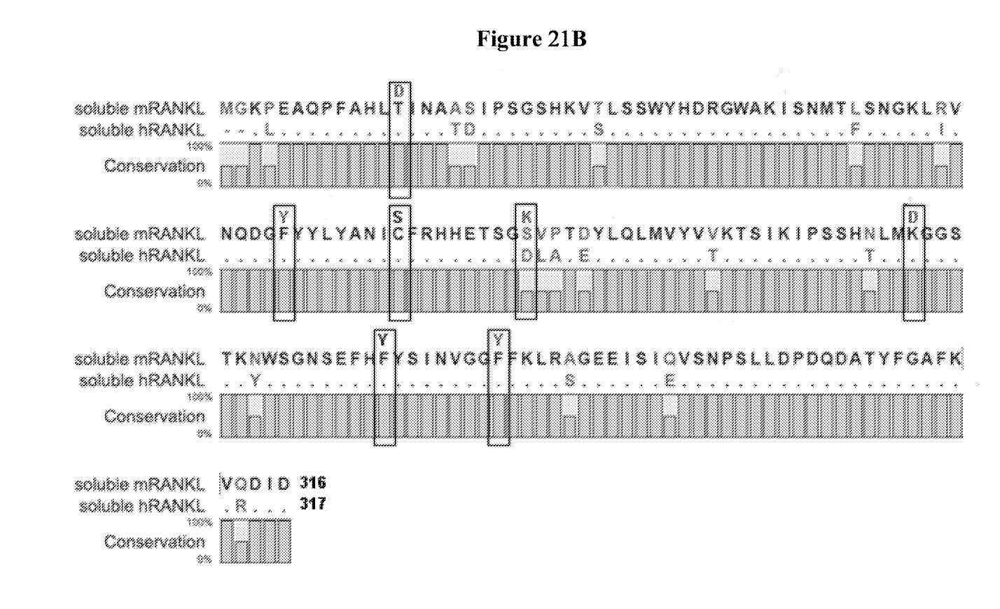

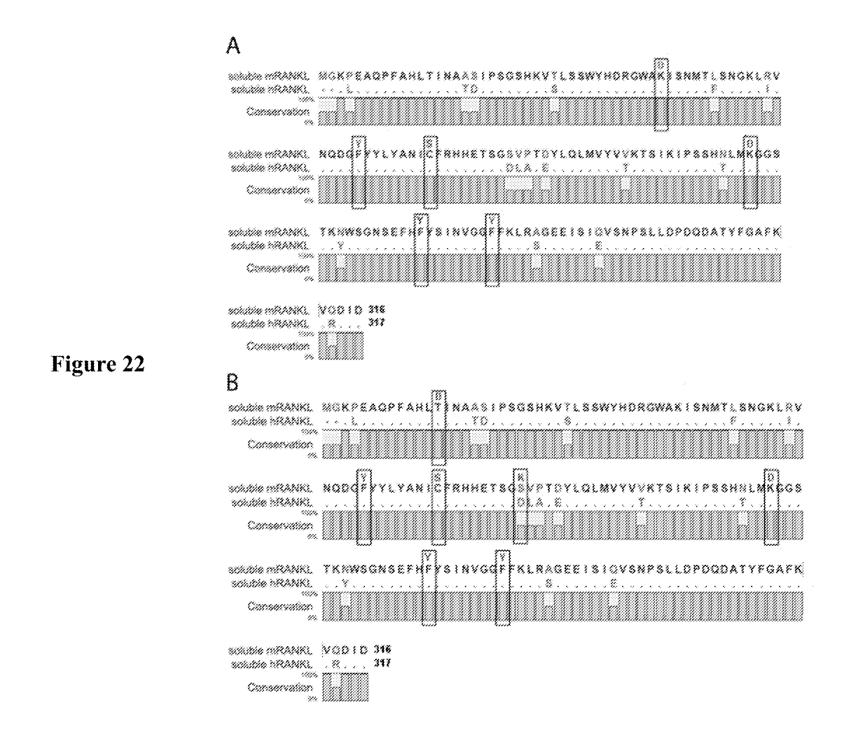

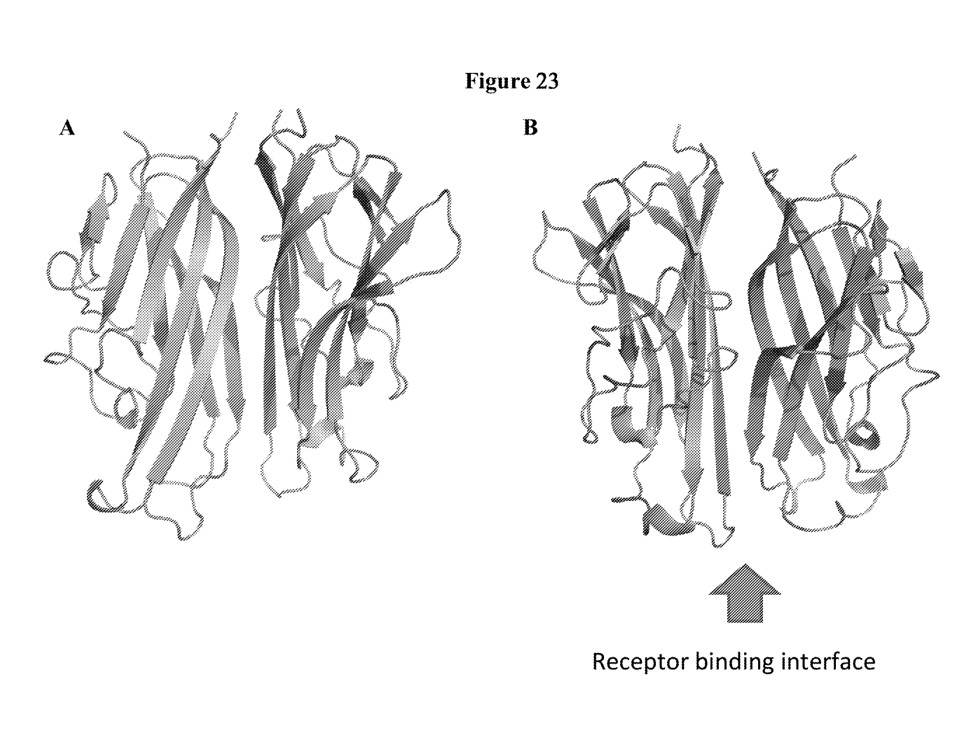

At day 7 osteoclastogenesis was determined using the tartrate resistance acid phosphatase (TRAP) assay (Sigma) according to manufacturer's instructions. The number of osteoclasts in each well was counted. The number of osteoclasts in the reference wells was set to one and the number of osteoclasts in the treated wells was calculated relative to the reference well. This allowed comparison between independent assays having different amount of cells at the start of the assay (FIGS. 9 and 19 B and C).

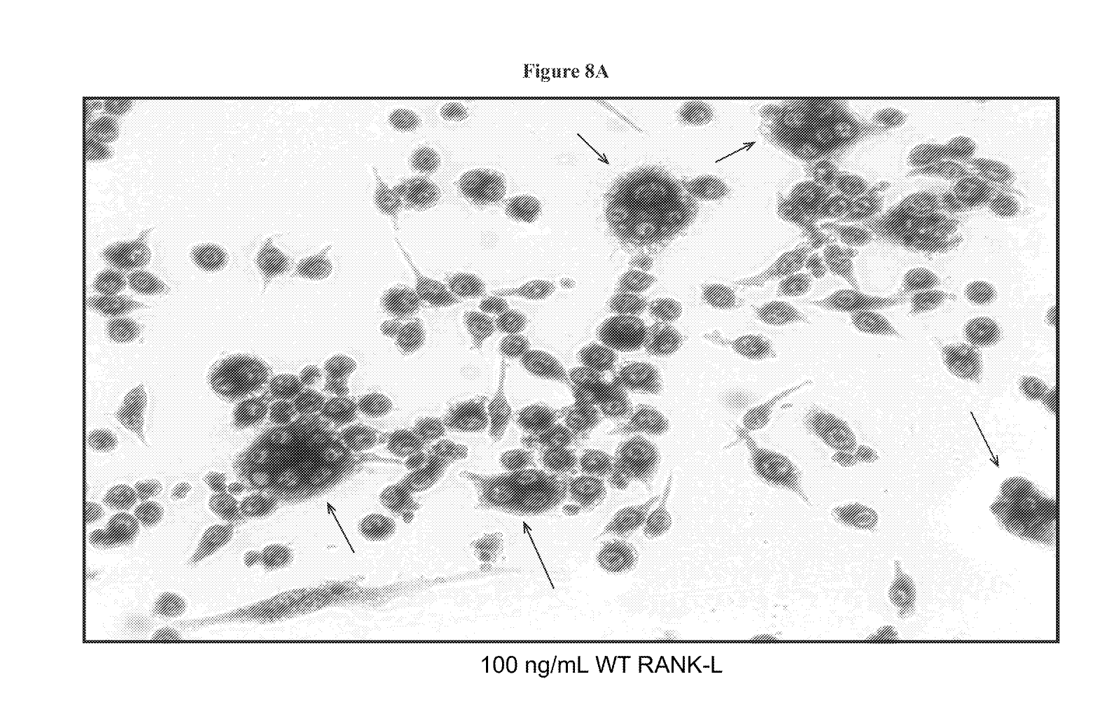

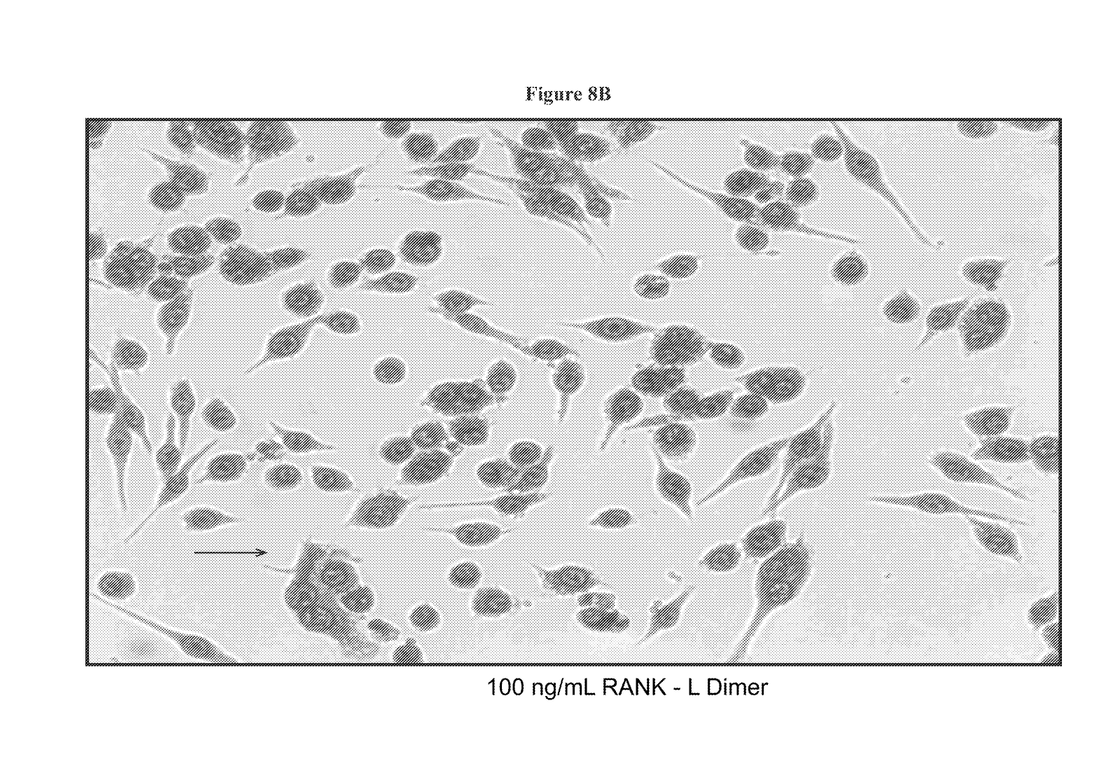

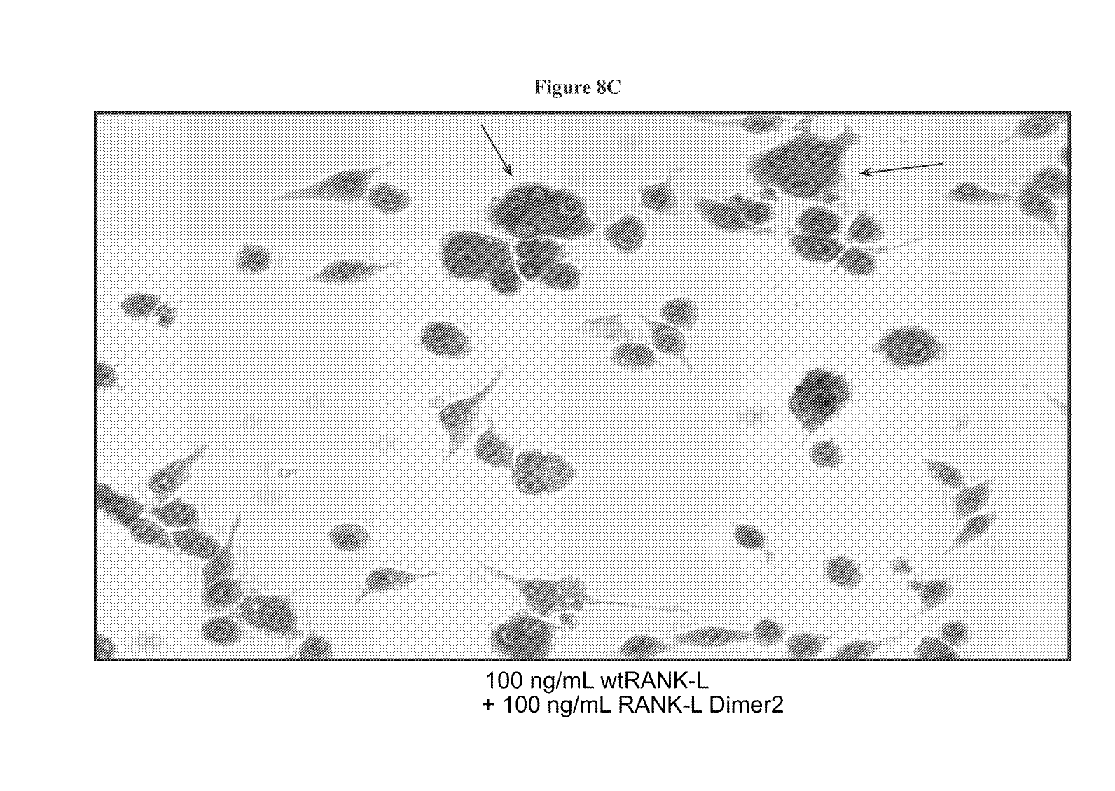

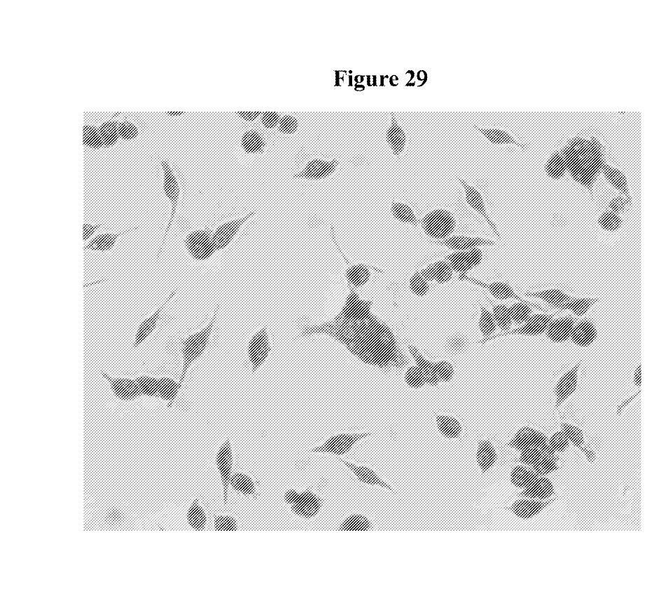

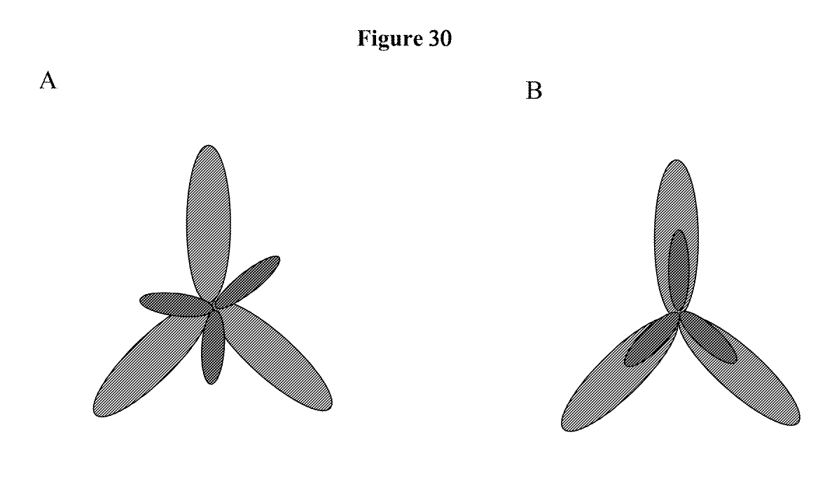

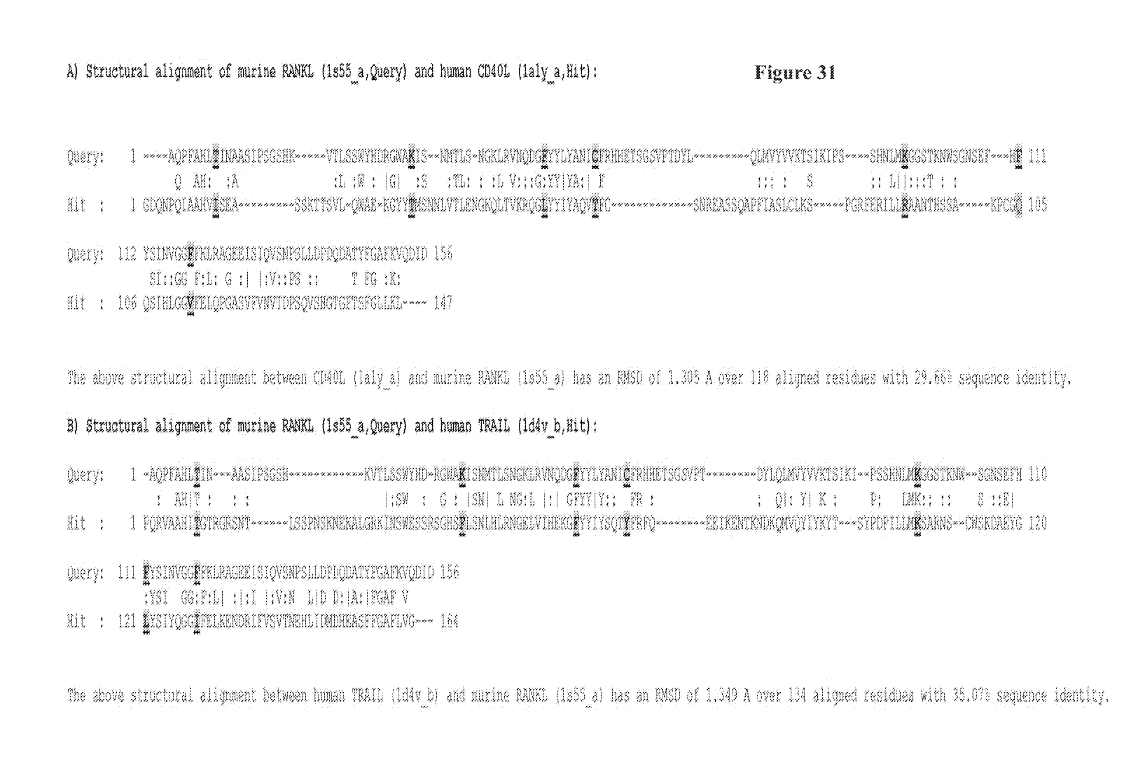

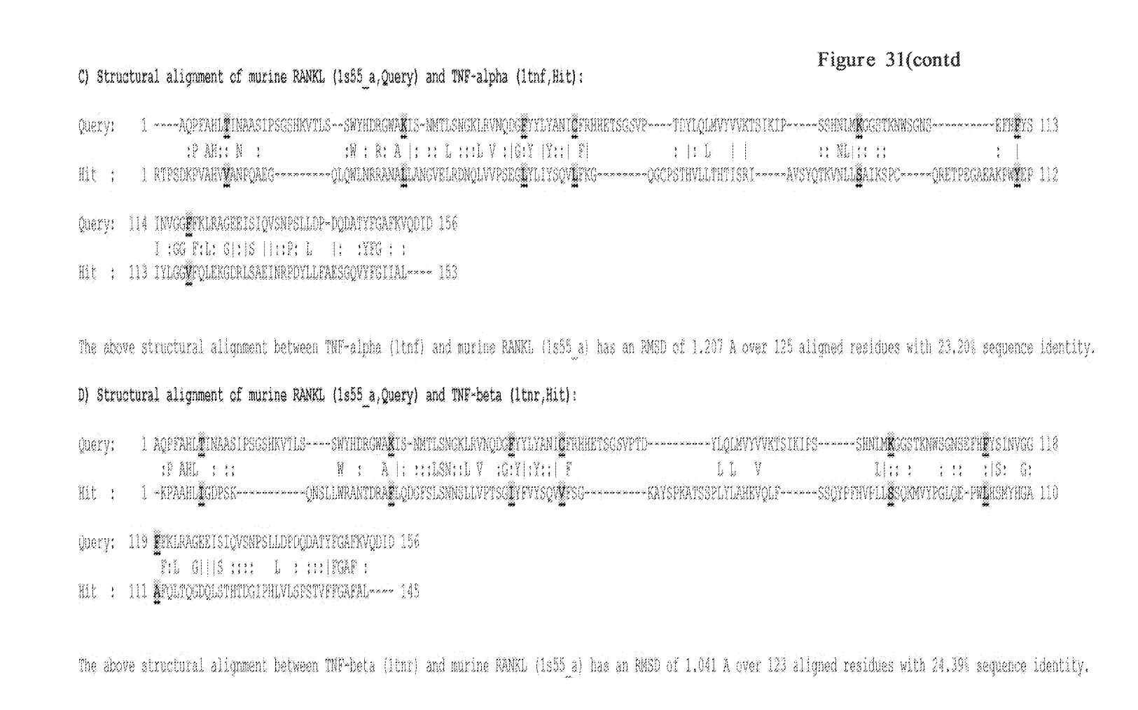

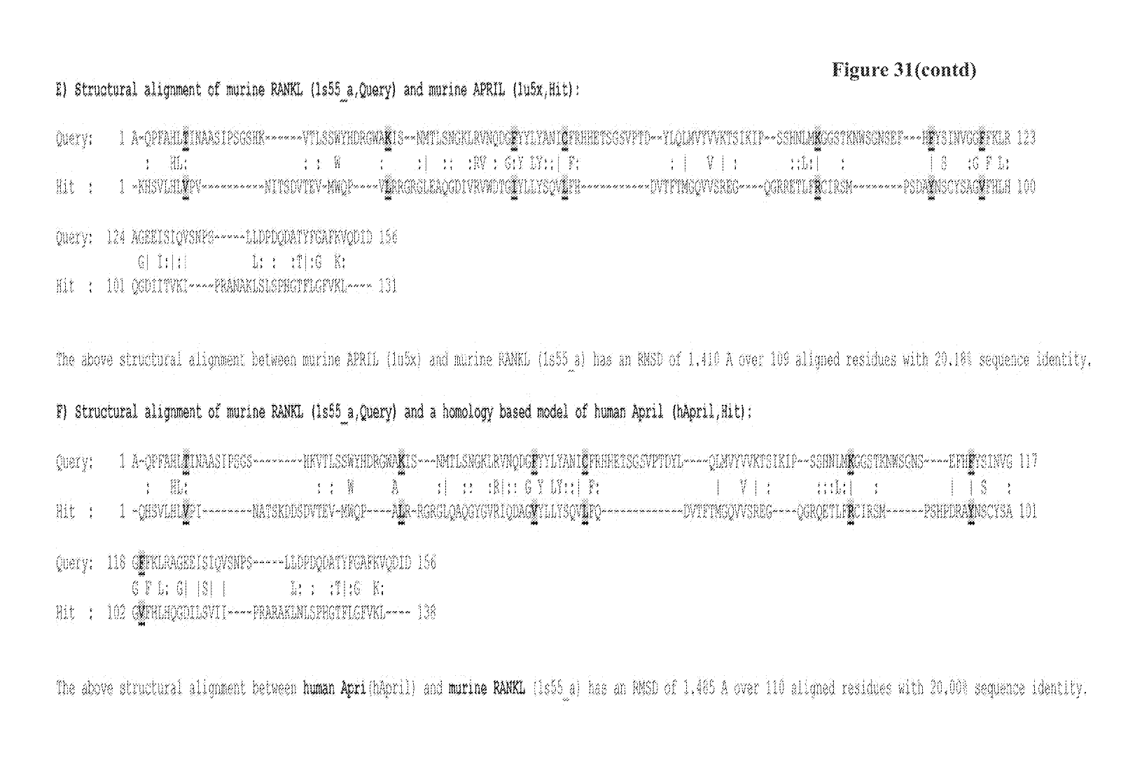

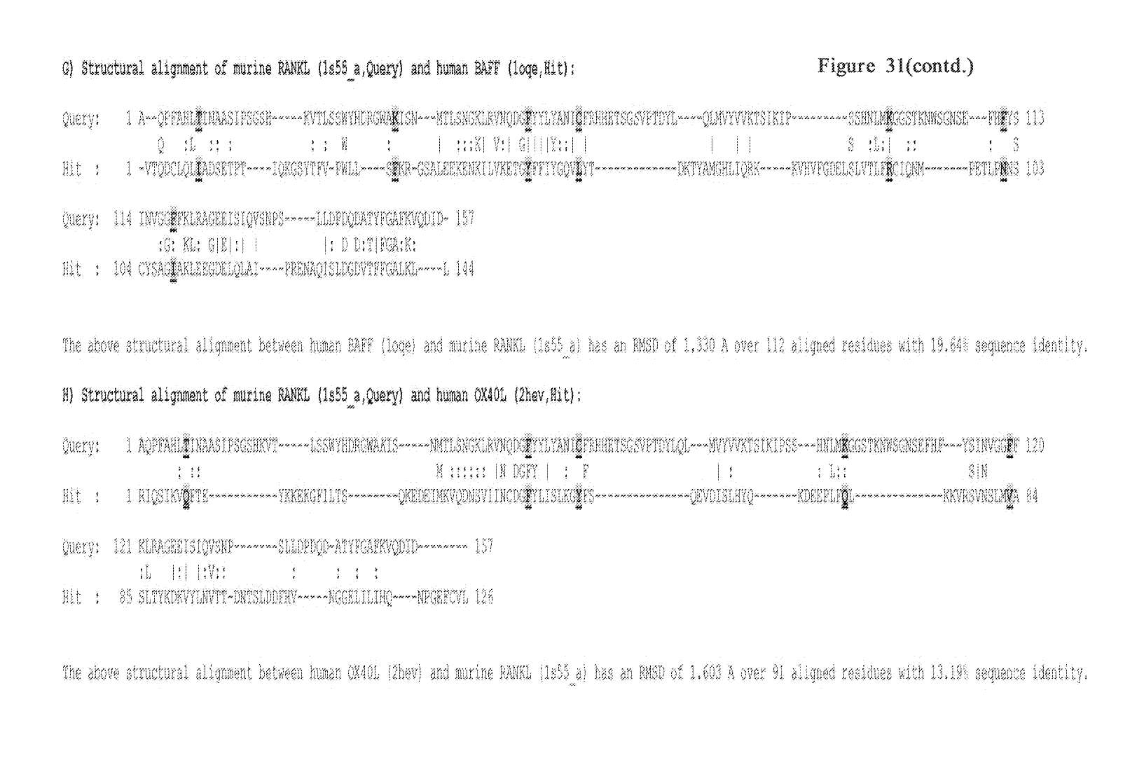

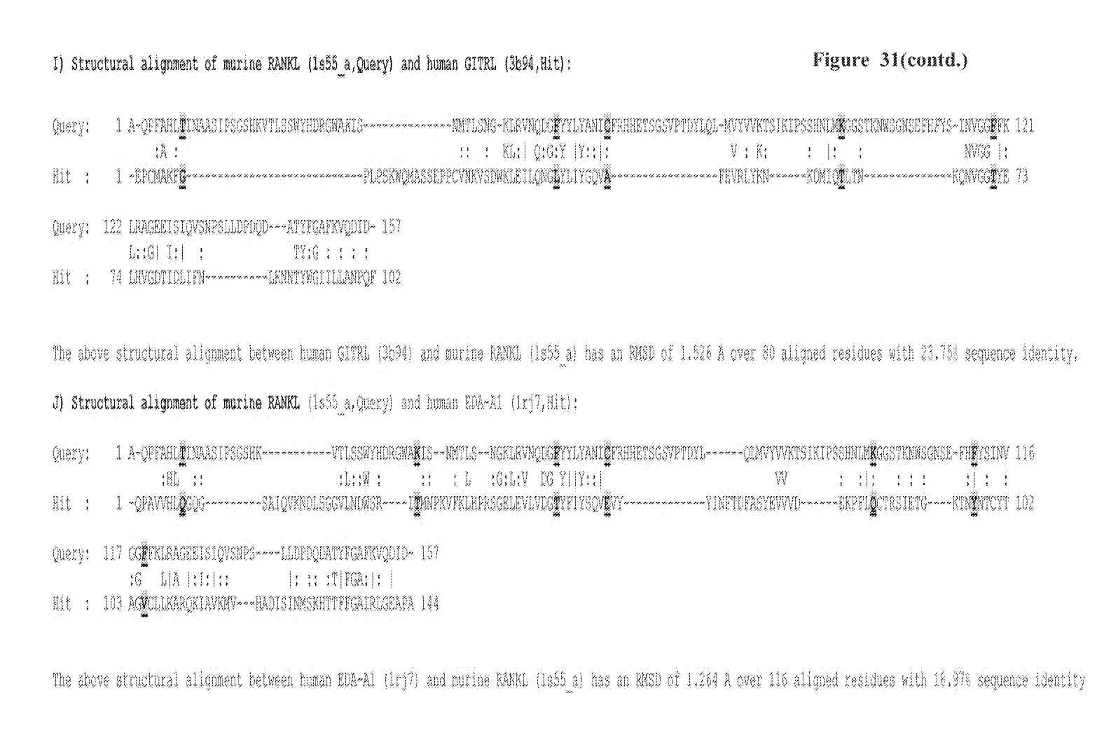

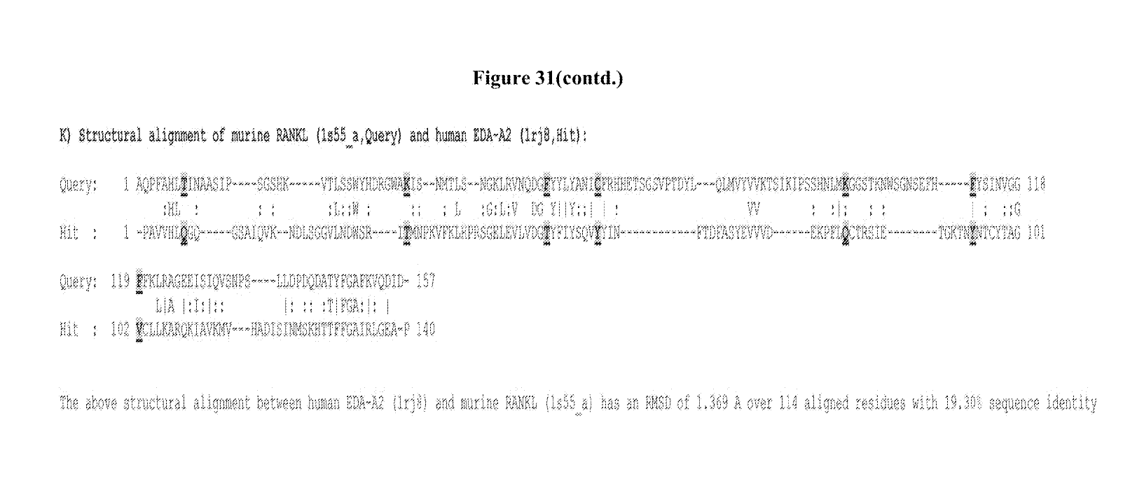

FIG. 8 shows the cells after staining with the multinucleated cells being marked. As can be seen, most multinucleated cells are present in the wild-type mRANKL treated sample. In contrast, the dimer treated samples show considerably less multinucleated cells and they are also less clustered than wild-type mRANKL treated cells. RANKL dimer 1 (mD1) (FIG. 29) appears to be less potent blocker of osteoclastogenesis than dimer 2 (mD2), the dimer 1 treated cells contain more nuclei and the osteoclasts are bigger than the ones observed in cells treated with RANKL dimer 2.

Upon treatment with RANKL dimer 1 and dimer 2 (100 ng/mL) the number of multinucleated cells remains similar to the untreated control samples. Importantly, treatment with RANKL dimers at a concentration of 100 ng/mL does not lead to an increase in the formation of multinucleated cells (FIG. 8B and FIGS. 19 B and C).

In conclusion, the RANKL dimers are potent inhibitors of wt-mRANKL induced osteoclastogenesis.

### Example 7

### Binding to Human RANK

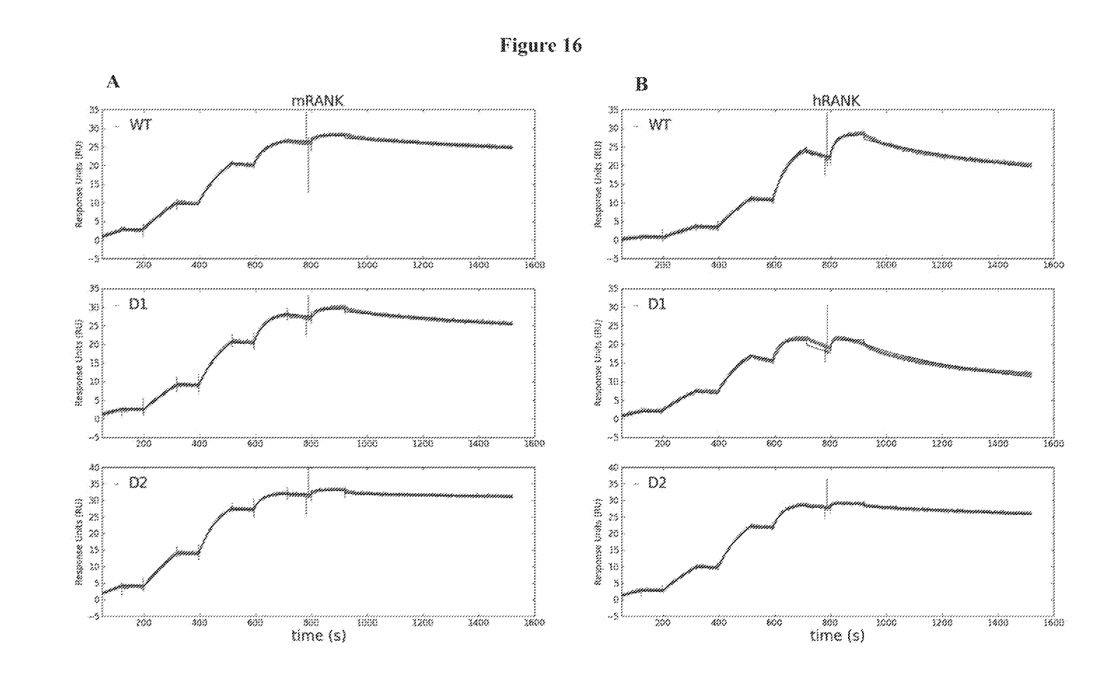

Considering the high similarity between human RANKL and murine RANKL (>88%) the murine dimers were also tested for hRANK binding. Binding to hRANK-Fc and mRANK-Fc (both from R&D Systems) was recorded for five consecutive concentrations of murine flag RANKL WT, D1 (mD1) or D2 (mD2) (˜0.3-15 nM) using SPR with a single cycle kinetics based approach. The capture level of mRANK-Fc and hRANK-Fc was low (20-40 RU) in order to ensure 1:1 binding. Both WT and the D1 and D2 dimeric variants bound with high affinity towards the mRANK receptor and to the human RANK receptor (FIG. 16: Table 8).

TABLE 8
binding of murine dimeric variants to human and murine RANK-Fc

| | mRANK-Fc | | | hRANK-Fc | | |
|---|---|---|---|---|---|---|
| | kₒₙ | k_{off} | K_{d} | kₒₙ | k_{off} | K_{d} |
| mWT | 6.14E+06 | 1.89E−04 | 3.15E−11 | 5.63E+06 | 5.75E−04 | 1.04E−10 |
| mD1 | 5.93E+06 | 2.47E−04 | 4.39E−11 | 8.80E+06 | 1.08E−03 | 1.29E−10 |
| mD2 | 6.73E+06 | 5.76E−05 | 8.55E−12 | 6.69E+06 | 1.73E−04 | 2.59E−11 |

Table 8 - Binding kinetics of D1, D2 and trimeric mRANKL WT to mRANK-Fc and hRANK as shown by SPR using single cycle kinetics. K_{d} in M, kₒₙ (1/Ms), k_{off} (1/s).

### Example 8

### Competitive Inhibition of RANK Binding

The ability of mD1 and mD2 to compete with mRANKL WT for binding to mRANK-Fc was investigated using a competitive ELISA. Serial dilutions of mD1 or mD2 (0-400 nM) and a fixed concentration (2 nM) of flag-mRANKL WT were added to mRANK-Fc coated wells. A dose-dependent decrease of flag-mRANKL WT binding was observed upon increasing concentrations of mD1 and mD2 (FIG. 18A). Both variants appear to be equally potent in blocking flag-mRANKL WT binding, having IC₅₀ values of 31 nM (±3 nM) (mD1) and 28 nM (±2 nM) (mD2).

As the concentration of flagRANKL WT used in the competitive ELISA was above its K_{D} for mRANK-Fc, for mD2 also an EC₅₀ shift experiment was performed. A dose-response curve of 0-50 nM flagRANKL WT binding to RANK-Fc was recorded in the presence and absence of various fixed concentrations of D2 (3, 5, 11, 22 or 43 nM) (FIG. 18B and FIG. 7). A clear right shift of the dose-response curve of flag-mRANKL WT was observed upon increasing concentrations of mD2 without any apparent loss in the maximal binding response. Upon a global fit of the individual dose response curves using a four parameter logistic model, an EC₅₀ for flag-mRANKL WT of 0.6 nM (±0.04 nM) was obtained and for mD2 an A₂ value of 4 nM (±0.5 nM) was calculated, i.e. the concentration of inhibitor required to shift the dose response curve by a factor of 2. This indicates that mD1 and mD2 are potent inhibitors.

### Example 9

### Design and Characterization of RANKL Heterodimeric Variants Based on Human RANKL

Based on the designs of murine based RANKL dimers that act as inhibitors of RANKL-signalling via the murine RANK receptor, similar variants were designed based on human RANKL that can act as inhibitors of RANKL-RANK signalling in humans. Also in view of a potential therapeutic application for these inhibitors, a variant based on human RANKL is highly desired.

Based on a high resolution crystal structure of murine RANKL (pdb code 1S55.pdb) a structural model of human RANKL was constructed using the protein design algorithm FoldX using a rotamer substitution approach as described before (refs #15, #28 and Reis C R et al Cell death & Disease 2010). Next, amino acid homologous to amino acid substitutions that were used to create the murine based dimers (table 2) were introduced in the structural model of human RANKL and the energetic effect on stability of dimer and trimer formation was assessed using FoldX (using a similar procedure as described in example 1). FoldX calculations revealed that all substitutions would be tolerated in the dimer and would disadvantage trimer formation. In addition to the two main variants (hD1.1 and hD2.1), two additional variants were designed for each main variant, comprising of either an additional I207R (hD1.2 and hD2.2) or I247E (hD1.3 and hD2.3) substitution in each monomer (table 9).

TABLE 9
human dimeric RANKL variants

| | Monomer | amino acid substitutions | | | | | |
|---|---|---|---|---|---|---|---|
| Dimer1 variants | | | | | | | |
| hD1-1 | A | K195D | C221S | F272Y | | | |
| | B | F213Y | K257D | F280Y | | | |
| hD1-2 | A | K195D | C221S | F272Y | I207R | | |
| | B | F213Y | K257D | F280Y | I207R | | |
| hD1-3 | A | K195D | C221S | F272Y | I247E | | |
| | B | F213Y | K257D | F280Y | I247E | | |
| Dimer2 variants | | | | | | | |
| hD2-1 | A | T169V | C221S | D230K | F272Y | | |
| | B | T169V | F213Y | D230K | K257D | F280Y | |
| hD2-2 | A | T169V | C221S | D230K | F272Y | | I207R |
| | B | T169V | F213Y | D230K | K257D | F280Y | I207R |
| hD2-3 | A | T169V | C221S | D230K | F272Y | | I247E |
| | B | T169V | F213Y | D230K | K257D | F280Y | I247E |

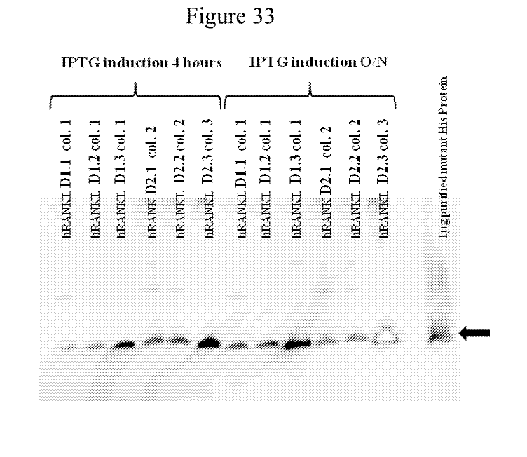

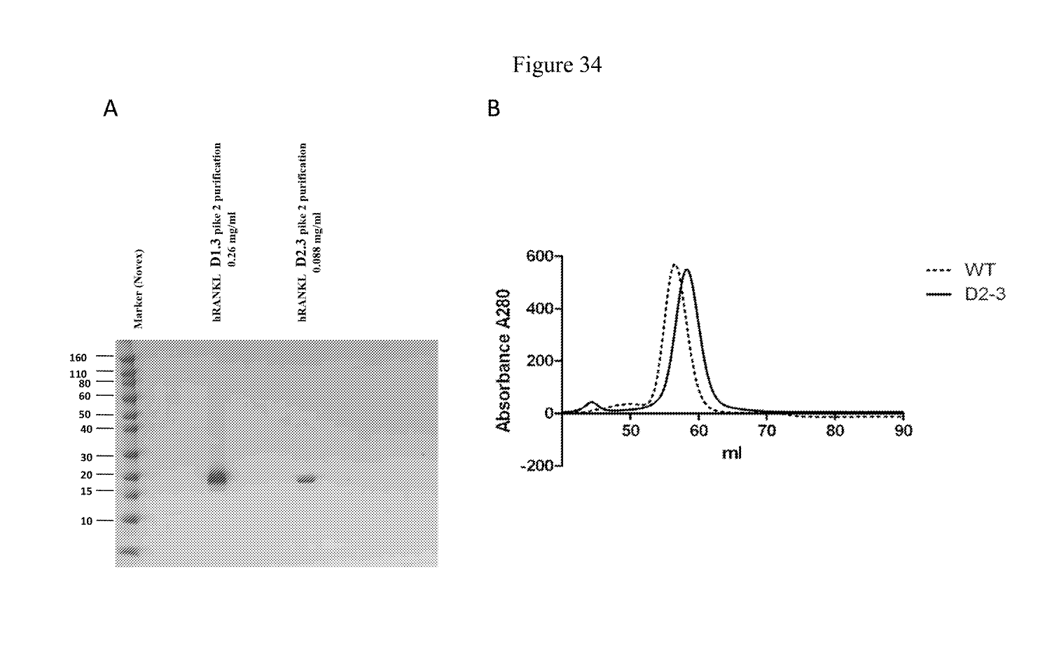

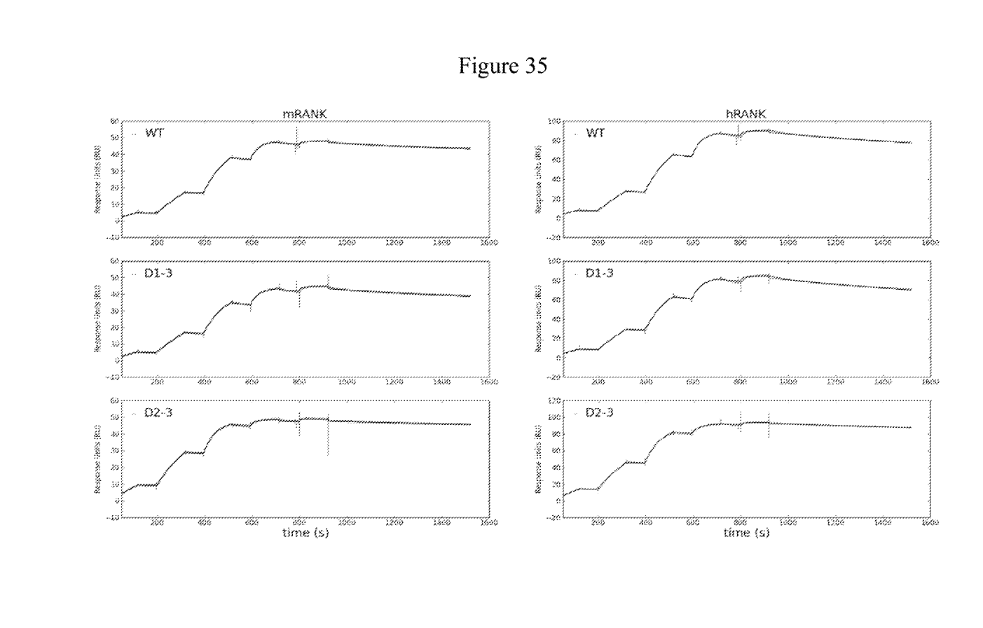

Synthetic constructs optimized for expression in E. coli were ordered from DNA 2.0 (Menlo Park, Calif., USA) encoding the first monomer under control of a T7 promoter and containing an N-terminal histag encoding sequence in frame with the first monomer (monomer A) followed by the second monomer (monomer B) under control of a second T7 promoter. The expression cassette of the vector is similar to the pCDF-Duet-1 bicistronic co-expression vector described in example 2 but the backbone of the vector is based on the pJexpress 411 protein expression vector of DNA 2.0 (Menlo Park, Calif., USA) which confers kanamycin resistance and uses a pUC origin of replication. The protein expression host E. coli BLR (DE3) was transformed with these constructs and soluble expression of the variants was detected after 4 hrs following induction with IPTG and after 16 hrs of induction with IPTG (figure X1) by Western Blot using an anti-his-tag antibody (Sigma). All variants showed over-expression of soluble human RANKL dimers (as detected by anti-his antibody) with variants hD1-3 and hD2-3 showing the highest yields (FIG. 33). Subsequently, variants hD1-3 and hD2-3 were expressed at 1 litre scale in 2×YT medium at 30° C. over night after induction with 1 mM IPTG. Cells were broken using French press and recombinant human RANKL dimers were purified from the soluble fraction using His-tag protein purification followed by size exclusion chromatography using the same protocol as for the murine dimers (see example 2). The purified dimers were over 90% pure as determined by SDS-PAGE Coomassie Brilliant blue staining (FIG. 34a). Comparison of the elution volumes of trimeric murine flagtag RANKL WT with hD2.3. on a Superdex 75 highload 16/60 size exclusion column (GE Lifesciences) revealed that the molecular weight of hD2.3 is substantial smaller than the flagmRANKL WT trimer (FIG. 34b), and consequently is also dimeric. The elution volume of hD1.3 is identical to that of hD2.3 and hence is also a dimer. A surface Plasmon based receptor binding assay revealed that hD1.3 and hD2.3 bind with high affinity towards human RANK and murine RANK and rate constants and affinity constants are similar to that of human RANKL WT (E. coli produced, R&D Systems) (FIG. 35 and table 10). Taken together, this shows that the dimeric variants based on human RANKL have similar properties as the murine RANKL based dimeric variants and hence can also be used to antagonize RANKL-RANK signalling.

TABLE 10
Binding kinetics of human RANKL dimers towards hRANK and mRANK

| | hRANK-Fc | | | mRANK-Fc | | |
|---|---|---|---|---|---|---|
| | kₐ (1/Ms) | k_{d} (1/s) | K_{D} (M) | kₐ (1/Ms) | k_{d} (1/s) | K_{D} (M) |
| hWT | 8.61E+05 | 2.30E−04 | 2.67E−10 | 1.04E+06 | 1.42E−04 | 1.36E−10 |
| hD1-3 | 7.98E+05 | 2.75E−04 | 3.45E−10 | 8.99E+05 | 1.90E−04 | 2.11E−10 |
| hD2-3 | 1.16E+06 | 8.87E−05 | 7.66E−11 | 1.65E+06 | 8.32E−05 | 5.04E−11 |

Table 10 - Binding kinetics of hD1.3, hD2.3 and trimeric human RANKL WT to mRANK-Fc and hRANK as shown by SPR using single cycle kinetics. K_{d} in M, k_{on (1/Ms), k}_{off} (1/s).

### Example 10

### FoldX Mutagenesis at Structurally Equivalent Positions in Other TNF-Ligands

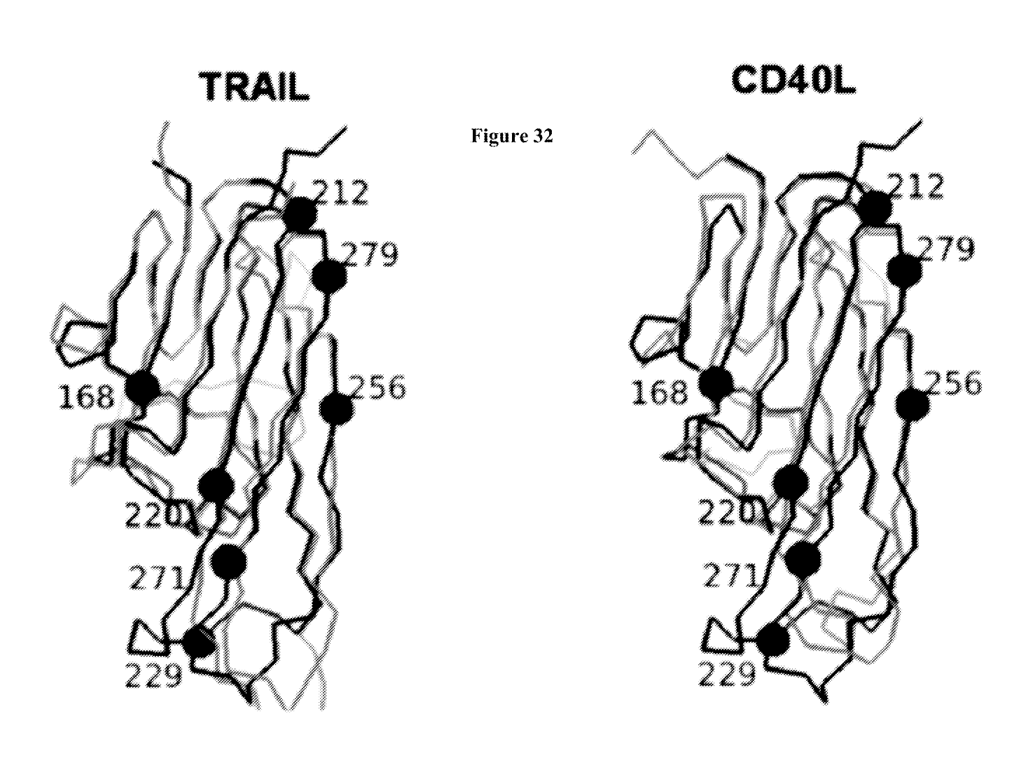

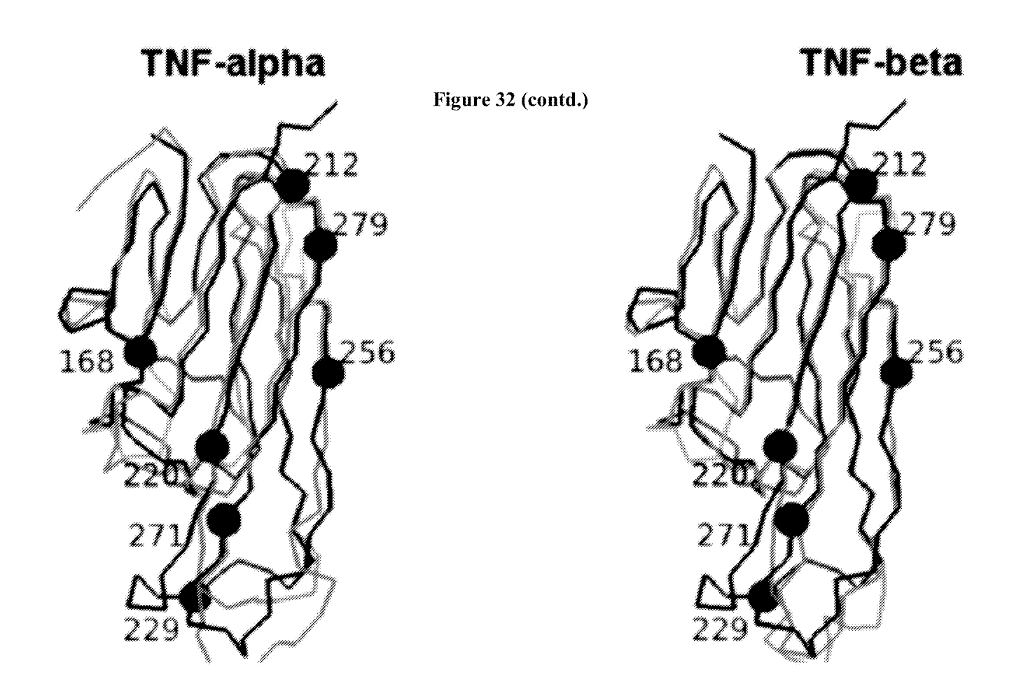

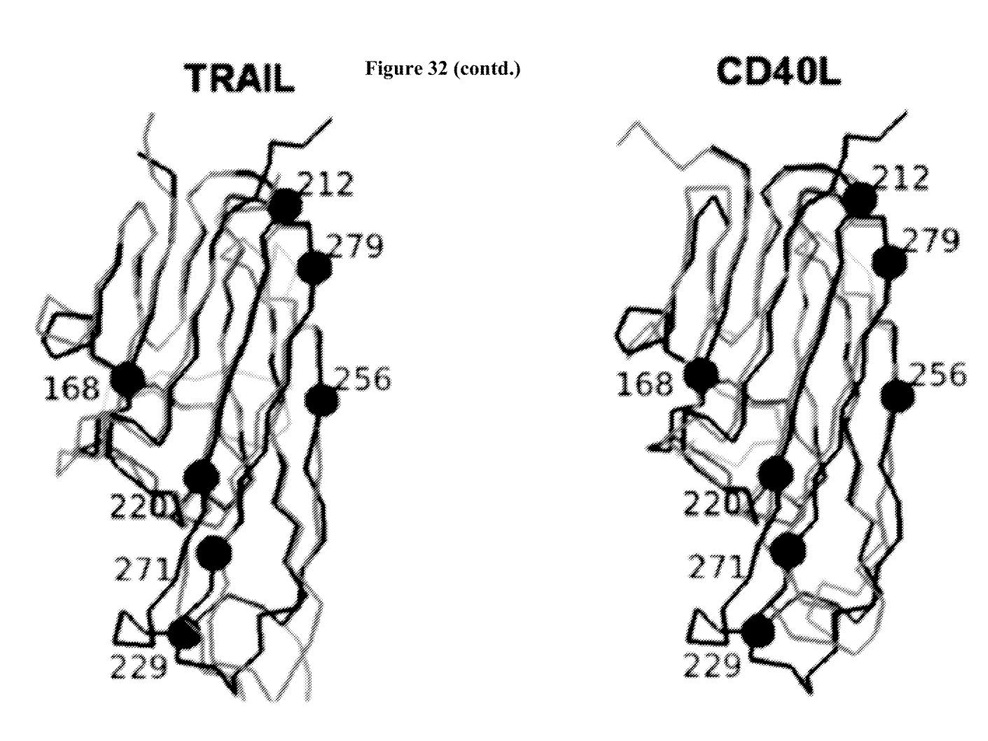

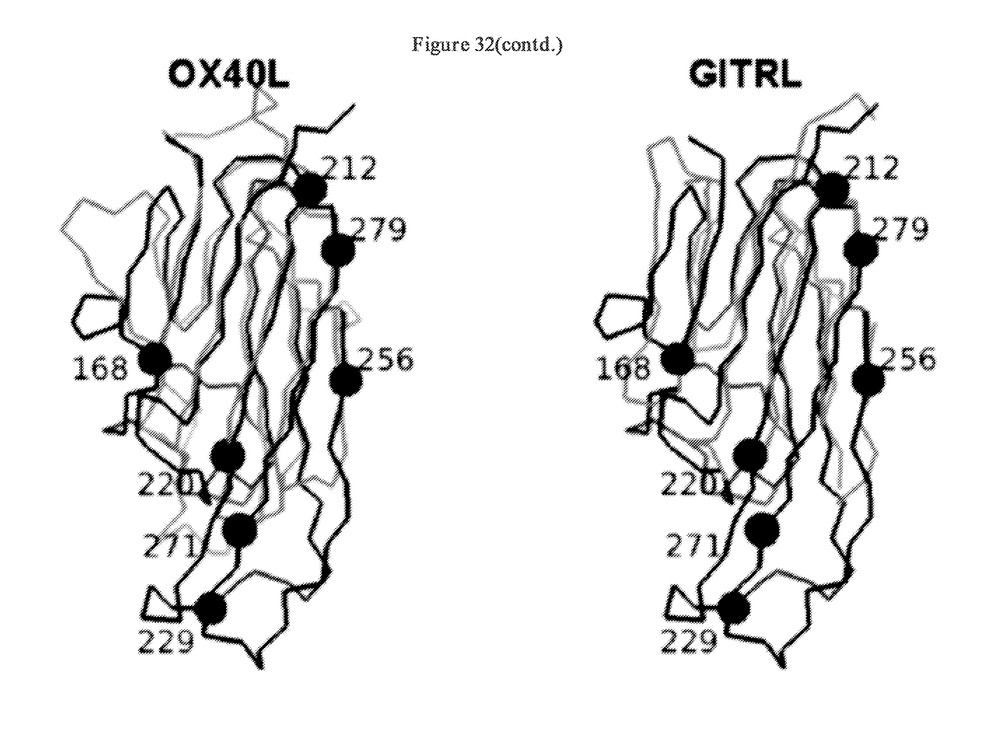

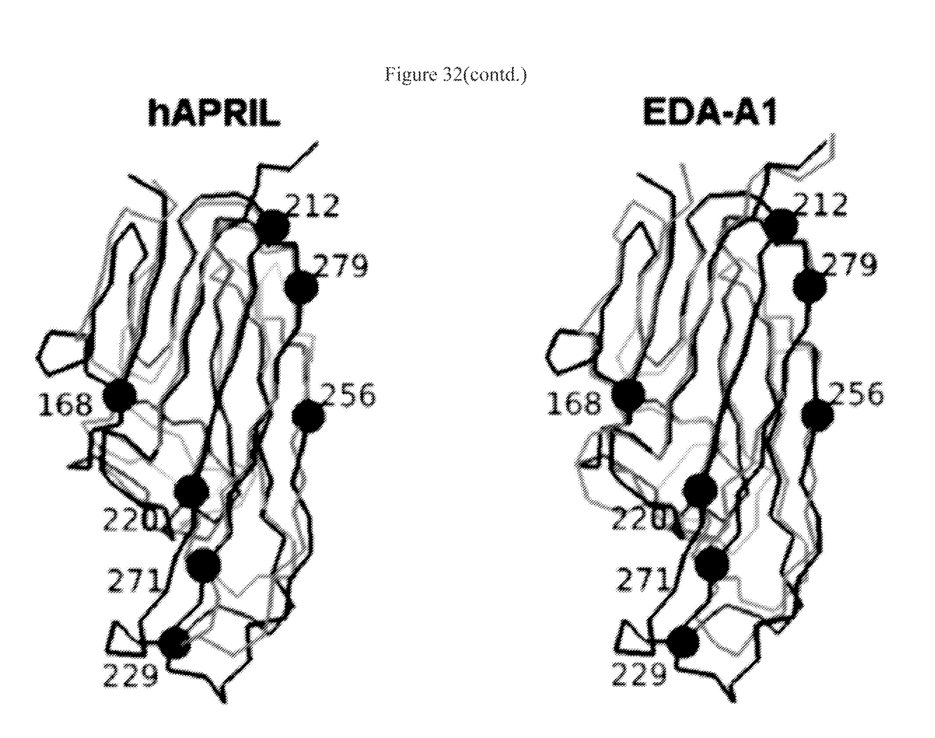

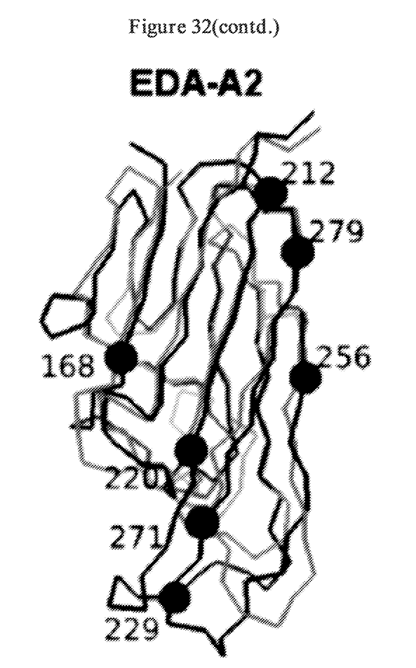

Given the structural conservation of TNF-ligand family members (FIG. 32) it can be expected that mutations at structurally equivalent positions will have similar effects on stability across the various ligands. Therefore, one might also expect that mutations at structurally equivalent positions (see Table 1 above) as those described in Tables 2 and 10 to create dimeric variants of murine and human RANKL (Table 2 and 10) will have a similar effect in other TNF-ligand family members.

The impact of amino acid substitutions at these structural equivalent positions on the structural stability of the TNF-ligand trimers was assessed with the protein design algorithm FoldX. The FoldX protein design algorithm has a highly accurate force-field to calculate changes in stability energy in proteins and it shows an excellent correlation with experimentally derived energetic parameters (24, 25). For that reason, it has been many times successfully used to improve stability or affinity of various proteins (15,26,27,28).

High resolution structures were downloaded from the protein database (PDB). The following x-ray structure files were used: 1S55 (murine RANKL); 1d4v (human TRAIL); 1kxg (human BAFF); 1xu1 (murine APRIL); 4tsv (human TNFα); 1tnr (human TNFβ); 1aly (human CD40L); 2hev (human OX40L) and; 2r32 (human GITRL). A homology based model of human RANKL was constructed using FoldX was constructed based on the murine structure. If necessary the biopolymer was reconstructed into the trimeric form using the transformations enclosed in the PDB file. Next, the structures were cleaned and repaired using FoldX. Afterwards, all structurally equivalent positions were mutated by FoldX to all 20 naturally occurring amino-acids and the stability energy due to the mutation was calculated. Selected mutations are depicted in Table 11: in general more polar amino acids were selected or amino acids of opposite charge. Although, for example F272Y or L164Y do not show a large increase in energy to destabilize the trimer, this mutation can be considered favorable as it will increase the solubility of a newly created dimer interface.

As can be observed in Table 11, at most of the positions an unfavorable effect on trimer stability is observed (DDG stability>0.5 kcal/mol). This is effect is even observed for structurally less similar TNF ligands that only share a part of the structurally equivalent positions (for example OX40L). Particular favorable structural equivalent positions are F279 and K256 (both mouse species) as these positions are present in all TNF ligand structures of varying degree of similarity and mutations at these position yield a substantial decrease in the calculated stability energy of the trimer structure. Of note, in this example no attempt was made to design fully stable dimers but to demonstrate the detrimental effect on trimer stability. For a proper design several of these mutations will need to be combined using a similar procedure as described in Example 1 and other parameters, such as solubility, might require additional optimization.

However, taken together, as in the case with murine and human RANKL the structural equivalent mutation positions can be used to convert trimeric TNF-ligands into dimeric variants.

TABLE 11
FoldX mutagenesis at structurally equivalent positions

| Muta- | | | | | | | | | | | | | | | | | | |
|---|---|---|---|---|---|---|---|---|---|---|---|---|---|---|---|---|---|---|
| tions | FoldX mutagenesis at Equivalent mutations positions in selected TNF family ligands | | | | | | | | | | | | | | | | | |
| position | (TRIMER stability in DDG (kcal/mol)) | | | | | | | | | | | | | | | | | |
| in | Human | | Human | | Human | | Murine | | | | | | Human | | Human | Human | | Human |
| murine | RANKL | | TRAIL | | BAFF | | APRIL | | TNFα | | TNFβ | | CD40L | | APRIL | OX40L | | GITRL |
| T168 | T169 | | T127 | | I150 | | V112 | | V93 | | I68 | | I127 | | V121 | Q65 | | G85 |
| K194 | K195 | 0.5 | F163D | 4.7 | F172D | 3.8 | L133D | 3.6 | L112D | 3.5 | F87D | 4.5 | T147D | 2.8 | L142 | | | |
| F212 | F213 | 0.8 | F181Y | 0.6 | Y192H | 2.7 | I153Y | 1.2 | L131Y | 0.7 | I106Y | 3.9 | L168Y | 1.9 | V162 | F100Y | 1.3 | L116 |
| C220 | C221 | 1.3 | Y189R | 3.1 | L200R | 1.7 | L161S | 3.6 | L139S | 3.0 | V114R | 4.4 | T176R | 7.0 | L170 | Y108S | 0.9 | A124 |
| S229 | D230 | | — | | — | | — | | — | | — | | — | | — | — | | |
| K256 | K257 | 4.5 | K224D | 4.1 | R231D | 3.0 | R186D | 2.0 | S171D | 0.9 | S150D | 2.1 | R207D | 1.5 | F194 | Q128D | 1.0 | T148 |
| F271 | F272 | 0.0 | L239R | 2.1 | N242 | | Y199H | 1.4 | Y191H | 2.8 | L164Y | 0.2 | Q220E | 1.1 | Y208 | — | | |
| F279 | F280 | 1.5 | I247Y | 10.9 | I250Y | 3.5 | V207Y | 3.2 | V199Y | 6.1 | A172Y | 19.3 | V228Y | 16.8 | V216 | V140Y | 9.0 | T161 |

### REFERENCES

1. Kanazawa, K., and Kudo, A. (2005) J. Bon. Min. Res., 20, 2053-2060
2. Parfitt, A. M. (2002) Bone, 30, 5-7
3. Teitelbaum, S. L. (2002) Science, 289, 1504-1508
4. Joseph Melton III, L. (2003) J. Bon. Min. Res., 18, 1139-1141
5. Boyce, B. F., and Xing, L. (2008) Arch. Biochem. Biophys., 474, 139-146
6. Anderson, D. M., Maraskovsky, E., Billingsley, W. L., Dougall, W. C., Tometsko, M. E., Roux, E. R., Teepe, M. C., DuBose, R. F., Cosman, D., Galibert, L. (1997) Nature, 390, 175-179
7. Wong, B. R., Rho, J., Arron, J., Robinson, E., Orlinick, J., Chao, M., Kalachikov, S., Cayani, E., Bartlett III, F. S., Frankel, W. N., Lee, S. Y., Choi, Y., J. B. C. (1997), 272, 25190-25194
8. Lacey, D. L., Timms, E., Tan, H. L., Kelley, M. J., Dunstan, C. R., Burgess, T., Elliott, R., Colombero, A., Elliott, G., Scully, S., Hsu, H., Sullivan, J., Hawkins, N., Davy, E., Capparelli, C., Eli, A., Qian, Y. X., Kaufman, S., Sarosi, I., Shalhoub, V., Senaldi, G., Guo, J., Delaney. J., Boyle, W. J. (1998) Cell, 93, 165-167
9. Wrigth, H. L., McCarhy, H. S., Middleton, J., Marshall, M. J. (2009) Curr Rev Musculoskelet Med, 2, 54-64
10. Ji, J.-D., Park-Min, K.-H., Shen, Z., Fajardo, R. J., Goldring, S. R., McHugh, K. P., Ivashkiv, L. B. (2009) J. Imm., 183, 7223-7233
11. Roato, I., Dámelio, P., Gorassini, E., Grimaldi, A., Bonello, L., Fiori, C., Delsedime, L., Tizzani, A., De Libero, A., Isaia, G., Ferracini, R., (2008) Plos One, 3, e3627
12. Mikami, S., Katsube, K., Oya, M., Ishida, M., Kosaka, T., Mizuno, R., Mochizuki, S., Ikeda, T., Mukai, M., Okada, Y. (2009) J. Pathol., 218, 530-539
13. Coleman, R. E. (2006) Clin. Canc. Res., 20s, 6243s-6249s
14. Kostenuit, P. J., Nguyen, H. Q., McCabe, J., Warmington K. S., Kurahara, C., Sun, N., Chen, C., Li, L., Cattley, R. C., Van, G., Scully, S., Elliott, R., Grisanti, M., Morony, S., Tan, H. L, Asuncion, F., Li, X., Ominsky, M. S., Stolina, M., Dwyer, D., Dougall, W. C., Hawkins, N., Boyle, W. J., Simonet, W. S., Sullivan J. K. (2009) J. Bon. Min. Res., 24, 182-195
15. Sloot, A. M. van der, Tur, V., Szegezdi, E., Mullaly, M. M., Cool, R. H., Samali, A., Serrano, L., Quax, W. J. (2006) PNAS, 103, 8634-8639
16. Ito, S., Hata, T., (2004) Vitam. Horm., 67, 19-33
17. Bossen, C., Ingold, K., Tardivel, A., Bodmer, J.-L., Gaide, O., Hertig, S., Ambrose, C., Tschopp, J., Schneider, P. (2006) J. B. C., 281, 13964-13971
18. Sambrook, J., Fritsch, E. F., Maniatis T. (1989) Molecular Cloning: A Laboratory Manual, 2^{nd} Ed., Cold Spring Harbor Laboratory, Cold Spring Harbor, N. Y.
19. Duan, L., Vos, P. de, Fan, M., Ren, Y. (2008) Front. Biosci., 13, 7064-7071
20. Walter, T. S., Liu, C., Huang P., Zhang, S., Wedderburn, L. R., Gao B., Owens, R. J., Stuart, D. I., Tang, P., Ren, J., (2009) Acta Cryst., F65, 597-600
21. Lewit-Bentley, A., Fourme, R., Kahn, R., Prange, T., Vachette, P., Tavernier, J., Hauquier, G., Niers, W., (1988) J. Mol Biol, 199, 389-392
22. Lam, J., Nelson, C. A., Ross, F. P., Teitelbaum, S. L., Fremont, D. H., (2001) JCI, 108, 971-979
24. Guerois, R., Nielsen, J. E., Serrano, L., (2002) J Mol Biol, 320, 369-387
25. Schymkowitz, J., Borg, J., Stricher, F., Nys, R., Rousseau, F., Serrano, L., (2005) Nucleic Acid Res., 33, W382-W388
26. Sloot, A. M. van der, Mullally, M. M., Fernandez-Ballester, G., Serrano, L., Quax, W. J., (2004) Prot. Eng. Des. Sel., 9, 673-680
27. Reis, C. R., Sloot A. M. van der, Szegezdi, E., Natoni, A., Tur, V., Cool, R. H., Samali, A., Serrano, L., Quax, W. J., (2009) Biochemistry, 10, 2180-2191
28. Tur, V., Sloot, A. M. van der, Reis, C. R., Szegzdi, E., Cool, R. H., Samali, A., Serrano, L., Quax, W. J., (2008) JBC, 29, 20560-20568
29. Nobbmann, U., Connah, M., Fish, B., Varley, P., Gee, C., Mulot, S., Chen, J., Zhou, L., Lu, Y., Sheng, F., Yi, J., Harding, S. E., (2007) Biotech. Gen. Eng. Rev., 24, 117-128
30. Reis, C. R., Assen, A. H. G. van, Quax, W. J., Cool, R. H., (2010) Mol Cell Prot, Sep. 17 (epub ahead of print)
31. Ta et al. (2010) PNAS, 47, 20281-20286
32. Sato et al. (2010) Current Opinions in Biotechnology, 17, 638-642
33. Locksley et al. (2001) Cell, 104, 487-501
34. Bodmer et al. (2002) Trends Biochem. Sci., 27, 19-26
35. Rieux-Laucat et al. (2003) Current Opinion in Immunology, 15, 325
36. Mackay and Ambrose (2003) Cytokine and growth factor reviews, 14, 311
37. Mackay and Kalled (2002) Current opinion in Immunology, 14, 783-790
38. Remington's Pharmaceutical Sciences
39. Peppel K et al. (1991) J Exp Med, 174(6), 1483-9
40. U.S. Pat. No. 5,447,851
41. Steed P M et al. (2003) Science, 301(5641), 1895-8
42. Bekker P J et al. (2004) J Bone Miner Res, 19(7), 1059-66
43. McClung M R et al. (2006) N Engl J Med, 354(8), 821-31
44. Clinical development of anti-RANKL therapy. Arthritis Research & Therapy 2007, 9(Suppl 1):S7.
45. Schramek et al. (2010) Nature, 468, 98-102
46. Gonzalez-Suarez et al. (2010) Nature, 468, 103-107
47. U.S. Pat. No. 7,381,792
48. Tan W et al. (2011) Nature, 470, 548-553
49. Jones D et al. (2006) Nature, 440, 692-696
50. Luo J et al. (2007) Nature, 446, 690-694
51. Oyajobi B et al. (2001) Cancer Res., 61, 2572-2578
52. Reis C et al. (2011) Mol Cell Proteomics, 10, M110 002808

1.-68. (canceled)

69. A variant of a TNF family ligand which is mutated such that it is not capable of assembling into a trimer,
wherein the variant ligand retains the ability to bind one or more of its cognate receptor(s), but wherein binding to the receptor does not activate the receptor.

70. The variant of a TNF family ligand of claim 69, wherein the variant does not homotrimerise with itself.

71. The variant of a TNF family ligand of claim 69, wherein the variant is capable of assembling into a dimer with another variant of the same ligand.

72. The variant of a TNF family ligand of claim 71, wherein the variant binds one or more of its cognate receptors within the cleft formed between two assembled ligand monomers, and wherein the TNF family ligand is optionally selected from the group consisting of RANKL, TRAIL, APRIL, BAFF, TNFalpha, CD30L, CD40L, FasL, Light, and Tweak.

73. The variant of a TNF family ligand of claim 69, wherein the variant comprises a mutation at one or more of positions 169, 195, 213, 230, 257, 272 and 280 in the human RANKL sequence, or an equivalent position as set out in Table 1 herein.

74. The variant of a TNF family ligand of claim 73, wherein the TNF variant comprises one or more of the mutations T169V, K195D, F213Y, D230K, K257D, F272Y and F280Y in the human RANKL sequence.

75. The variant of a TNF family ligand of claim 74, wherein the TNF variant further comprises a mutation at one or more of positions 207, 221 and 247 in the human RANKL sequence, and wherein the TNF variant optionally comprises one or more of the mutations I207R, C221S, C221A and I247E in the human RANKL sequence.

76. The variant of a TNF family ligand of claim 74, wherein the variant comprises the mutations:
(a) K195D, C221S and F272Y in the human RANKL sequence;
(b) F213Y, K257D and F280Y in the human RANKL sequence;
(c) K195D, I207R, C221S and F272Y in the human RANKL sequence;
(d) I207R, F213Y, K257D and F280Y in the human RANKL sequence;
(e) K195D, C221S, I247E and F272Y in the human RANKL sequence;
(f) F213Y, I247E, K257D and F280Y in the human RANKL sequence;
(g) T169V, D230K and F272Y in the human RANKL sequence;
(h) T169V, F213Y, C221A, D230K, K257D and F280Y in the human RANKL sequence;
(i) T169V, C221S, D230K and F272Y in the human RANKL sequence;
(j) T169V, F213Y, D230K, K257D and F280Y in the human RANKL sequence;
(k) T169V, I207R, C221S, D230K and F272Y in the human RANKL sequence;
(l) T169V, I207R, F213Y, D230K, K257D and F280Y in the human RANKL sequence;
(m) T169V, C221S, D230K, I247E and F272Y in the human RANKL sequence; or
(n) T169V, F213Y, D230K, I247E, K257D and F280Y in the human RANKL sequence.

77. The variant of a TNF family ligand of claim 69, wherein the variant is not capable of assembling into a dimer with the same or other TNF family ligands, and the variant optionally binds its cognate receptor on the solvent exposed surface of the TNF ligand variant, wherein said variant is optionally selected from the group consisting of APRIL and BAFF.

78. The variant of a TNF family ligand of claim 69, wherein:
(a) the variant has an increased binding affinity for one or more of its cognate receptor(s), compared to the wild-type TNF family ligand;
(b) the variant of a TNF family ligand has a decreased binding affinity for one or more of its cognate non-target receptor(s), compared to the wild-type TNF family ligand; and/or
(c) the variant is soluble.

79. A dimer comprising two variants of a TNF family ligand of claim 69, wherein the dimer is optionally a heterodimer.

80. A complex comprising one or more of the variant of a TNF family ligand of claim 69 and one or more cognate receptors for the TNF family ligand, wherein the complex optionally comprises two variants of a TNF family ligand of claim 69.

81. A complex comprising a dimer of claim 79 and one or more cognate receptors for the TNF family ligand, wherein the complex optionally comprises two cognate receptors for the TNF family ligand.

82. A nucleotide sequence encoding the variant of a TNF family ligand of claim 69.

83. A vector comprising the nucleotide sequence of claim 82.

84. A host cell comprising the nucleotide sequence of claim 82.

85. A pharmaceutical composition comprising the variant of a TNF family ligand of claim 69.

86. A method of treating osteoporosis, rheumatoid arthritis, Paget's disease, malignancy induced bone disease or cancer comprising administering a pharmaceutically effective amount of the variant of a TNF family ligand of claim 69 to a patient in need of treatment.

87. A transgenic animal which expresses the variant of a TNF family ligand of claim 69.

88. A method for producing a variant of a TNF family ligand comprising the steps of:
a) identifying amino acids in the TNF family ligand that are located in the trimerisation interface as candidates for mutation;
b) substituting each of one or more residues in the trimerisation interface; and
c) selecting amino acid substitutions which have a neutral or positive effect on the stability of the dimer but a negative effect on the stability of the trimer,
wherein the method optionally comprises one or more of the steps of:
d) selecting amino acid substitutions to increase the affinity for one or more of the target receptor(s);
e) selecting amino acid substitutions to increase selectivity for one or more of the target receptors, either by increasing affinity for one or more of the target receptor(s) or decreasing affinity for one or more of the decoy receptors.
f) producing a variant of a TNF family ligand; and
g) modifying the variant to improve its properties such as to decrease its immunogenicity or improve its pharmacokinetics. Such modifications may include one or more of pegylation, acetylation, formylation, alkylation such as methylation, and glycosylation,
and wherein the variant of a TNF family ligand is optionally an inhibitory variant of a TNF family ligand.
